(19) United States
(12) Patent Application Publication
(10) Pub. No.: US 2025/0197483 A1
(43) Pub. Date: Jun. 19, 2025

(54) NEUTRALIZING ANTIBODIES TO HIV-1 ENV AND THEIR USE

(71) Applicant: The United States of America, as represented by the Secretary, Department of Health & Human Services, Bethesda, MD (US)

(72) Inventors: Peter D. Kwong, Washington, DC (US); John R. Mascola, Rockville, MD (US); Young Do Kwon, Rockville, MD (US); Amarendra Pegu, Rockville, MD (US); Eun Sung Yang, Bethesda, MD (US); Krisha McKee, Hanover, PA (US); Nicole Doria-Rose, Garrett Park, MD (US)

(73) Assignee: The United States of America, as represented by the Secretary, Department of Health & Human Services, Bethesda, MD (US)

(21) Appl. No.: 18/849,734

(22) PCT Filed: Mar. 28, 2023
(86) PCT No.: PCT/US2023/065068

§ 371 Date: Sep. 23, 2024

Related U.S. Application Data
(60) Provisional application No. 63/324,152, filed on Mar. 28, 2022.

(51) Int. Cl. C07K 16/10; A61K 39/00; A61P 31/18; G01N 33/569

(52) U.S. Cl. CPC C07K 16/1063; A61P 31/18; G01N 33/56988; A61K 2039/505; C07K 2317/76; C07K 2317/92; C07K 2317/94; G01N 2333/162

(57) ABSTRACT

Antibodies and antigen binding fragments that specifically bind to HIV-1 Env and neutralize HIV-1 are disclosed. Nucleic acids encoding these antibodies, vectors and host cells are also provided. Methods for detecting HIV-1 using these antibodies are disclosed. In addition, the use of these antibodies, antigen binding fragment, nucleic acids and vectors to prevent and/or treat an HIV-1 infection is disclosed.

### CROSS REFERENCE TO RELATED APPLICATIONS

This application is the U.S. National Stage of International Application No. PCT/US2023/065068, filed Mar. 28, 2023, which was published in English under PCT Article 21(2), which in turn claims priority to U.S. Provisional Application No. 63/324,152, filed Mar. 28, 2022. The provisional application is incorporated by reference herein in its entirety.

### SEQUENCES

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. The Sequence Listing is submitted as an XML file in the form of the file named “4239-108033-04_Sequence_Listing” (-169,674 bytes), which was created on Mar. 28, 2023 which is incorporated by reference herein.

### FIELD OF THE DISCLOSURE

This relates to monoclonal antibodies and antigen binding fragments that specifically bind to HIV-1 Env and their use, for example, in methods of treating a subject with HIV-1 infection.

### BACKGROUND

Human Immunodeficiency Virus type 1 (HIV-1) infection, and the resulting Acquired Immunodeficiency Syndrome (AIDS), remain threats to global public health, despite extensive efforts to develop anti-HIV-1 therapeutic agents.

An enveloped virus, HIV-1 hides from humoral recognition behind a wide array of protective mechanisms. The major HIV-1 envelope protein (HIV-1 Env) is a glycoprotein of approximately 160 kD (gp160). During infection, proteases of the host cell cleave gp160 into gp120 and gp41. Together gp120 and gp41 make up the HIV-1 envelope spike, which interacts with the host-cell receptor CD4 to facilitate virus infection, and is a target for neutralizing antibodies.

Neutralizing antibodies that bind to HIV-1 Env have been identified, including VRC01, which is the prototypical member of the “VRC01-class” of antibodies that specifically bind to the CD4-binding site of HIV-1 Env and neutralize a high percentage of HIV-1 strains. Non-limiting examples of VRC01-class antibodies include N6 and VRC07-523. Another broadly neutralizing antibody to HIV-1 is 10E8, which specifically binds to gp41 at the base of the HIV-1 Env ectodomain at a site called the membrane-proximal external region.

However, there is a need to develop additional broadly neutralizing antibodies for HIV-1 that are not auto-reactive and have increased half-life for prevention or treatment of HIV-1.

### SUMMARY

Disclosed herein are modified forms of broadly neutralizing antibodies to HIV-1 that maintain neutralization potency and breadth, are not autoreactive, and have increased half-life.

In some implementations, a monoclonal antibody is provided comprising a heavy chain variable region (V_{H}) and a light chain variable region (V_{L}) comprising amino acid sequences respectively set forth as SEQ ID NOs: 1 and 5, respectively (VRC07-523LS.v11), SEQ ID NOs: 7 and 5, respectively (VRC07-523LS.v14), EQ ID NOs: 9 and 10, respectively (VRC07-523LS.v21), EQ ID NOs: 13 and 14, respectively (VRC07-523LS.v26), SEQ ID NOs: 17 and 14, respectively (VRC07-523LS.v32), SEQ ID NOs: 17 and 10, respectively (VRC07-523LS.v34), SEQ ID NOs: 59 and 10, respectively (VRC07-523LS.cv34), SEQ ID NOs: 23 and 24, respectively (N6LS.C1), SEQ ID NOs: 19 and 27, respectively (N6LS.15), SEQ ID NOs: 29 and 30, respectively (N6LS.30), SEQ ID NOs: 29 and 27, respectively (N6LS.35), SEQ ID NOs: 19 and 33, respectively (N6LS.47), SEQ ID NOs: 35 and 33, respectively (N6LS.49), SEQ ID NOs: 37 and 33, respectively (N6LS.51), SEQ ID NOs: 39 and 33, respectively (N6LS.58), SEQ ID NO: 23 and 33, respectively (N6LS.ATS8), SEQ ID NOs: 45 and 42, respectively (10E8v4-5RLS.C12), SEQ ID NOs: 45 and 47, respectively (10E8v4-5RLS.C13), SEQ ID NOs: 45 and 49, respectively (10E8v4-5RLS.C14), SEQ ID NOs: 51 and 52, respectively (10E8v4-5RLS.C15), SEQ ID NOs: 53 and 42, respectively (10E8v4-5RLS.C18), SEQ ID NOs: 55 and 42, respectively (10E8v4-5RLS.C24), SEQ ID NO: 65 and 66, respectively (10E8v4-5RLS.C27), SEQ ID NO: 65 and 69, respectively (10E8v4-5RLS.C30), SEQ ID NO: 98 and 66, respectively (10E8.ATS13), SEQ ID NO: 77 and 2, respectively (VRC01.23LS.cv1), SEQ ID NO: 59 and 10, respectively (VRC01.23LS.cv34), or SEQ ID NO: 77 and 10, respectively (VRC01.23LS.ATS5). The monoclonal antibody or antigen binding fragment specifically binds to HIV-1 Env and neutralizes HIV-1. In several implementations, the monoclonal antibody or antigen binding fragment specifically binds to HIV-1 Env, neutralizes HIV-1 Env, is not autoreactive, and has improved in vivo half-life.

In some implementations, a monoclonal antibody is provided comprising a heavy chain and a light chain comprising amino acid sequences set forth as SEQ ID NOs: 3 and 6, respectively (VRC07-523LS.v11), SEQ ID NOs: 8 and 6, respectively (VRC07-523LS.v14), SEQ ID NOs: 11 and 12, respectively (VRC07-523LS.v21), SEQ ID NOs: 15 and 16, respectively (VRC07-523LS.v26), SEQ ID NOs: 18 and 16, respectively (VRC07-523LS.v32), SEQ ID NOs: 18 and 12, respectively (VRC07-523LS.v34), SEQ ID NOs: 60 and 61, respectively (VRC07-523LS.cv34), 5 SEQ ID NOs: 25 and 26, respectively (N6LS.C1), SEQ ID NOs: 21 and 28, respectively (N6LS.15), SEQ ID NOs: 31 and 32, respectively (N6LS.30), SEQ ID NOs: 31 and 28, respectively (N6LS.35), SEQ ID NOs: 21 and 34, respectively (N6LS.47), SEQ ID NOs: 36 and 34, respectively (N6LS.49), SEQ ID NO: 64 and 97, respectively (N6LS.cv49), SEQ ID NOs: 38 and 34, respectively (N6LS.51), SEQ ID NOs: 40 and 34, respectively (N6LS.58), SEQ ID NO: 86 and 87, respectively (N6LS.ATS8), SEQ ID NOs: 46 and 44, respectively (10E8v4-5RLS.C12), SEQ ID NOs: 46 and 48, respectively (10E8v4-5RLS.C13), SEQ ID NOs: 46 and 50, respectively (10E8v4-5RLS.C14), SEQ ID NOs: 52 and 44, respectively (10E8v4-5RLS.C15), SEQ ID NOs: 53 and 44, respectively (10E8v4-5RLS.C18), SEQ ID NOs: 56 and 44, respectively (10E8v4-5RLS.C24), SEQ ID NO: 67 and 68, respectively (10E8v4-5RLS.C27), SEQ ID NO: 67 and 70, respectively (10E8v4-5RLS.C30), SEQ ID NO: 75 and 76, respectively (10E8v4-5RLS.cv30), SEQ ID NO: 94 and 93, respectively (10E8.ATS13), SEQ ID NO: 78 and 58, respectively (VRC01.23LS.cvl), SEQ ID NO: 60 and 61, respectively (VRC01.23LS.cv34), SEQ ID NO: 96 and 83, respectively (VRC01.23LS.ATS5), SEQ ID NOs: 57 and 58, respectively (VRC07-523LS.cv1), SEQ ID NOs: 62 and 63, respectively (N6LS.cv1), SEQ ID NO: 43 and 71, respectively (10E8v4.cc11), SEQ ID NO: 72 and 44, respectively (10E8v4.ccl6),or SEQ ID NO: 73 and 74, respectively (10E8v4-5RLS.cvl).

In several aspects, the monoclonal antibody further comprises an alpha-synuclein (ATSα) domain comprising or consisting of the amino acid sequence set forth as DPDNEAYEMPSEEGYQDYEPEA (SEQ ID NO: 99) fused to the C-terminus of the light chain, the C-terminus of the heavy chain, or both the C-terminus of the light chain and the C-terminus of the heavy chain. ATSα.

In additional implementations, an antigen binding fragment of the monoclonal antibody is provided.

Also disclosed are compositions including the antibodies and antigen binding fragments, as well as related nucleic acid molecules and expression vectors.

The disclosed antibodies and antigen binding fragments potently neutralize HIV-1 in an accepted in vitro model of HIV-1 infection. Accordingly, a method is disclosed for inhibiting an HIV-1 infection in a subject, comprising administering a therapeutically effective amount of one or more of the disclosed antibodies, antigen binding fragments, nucleic acid molecules, vectors, or compositions, to the subject, wherein the subject is at risk of or has an HIV-1 infection.

The antibodies, antigen binding fragments, nucleic acid molecules, vectors, and compositions disclosed herein can be used for a variety of additional purposes, such as for detecting an HIV-1 infection or diagnosing HIV-1 infection in a subject, or detecting HIV-1 in a sample.

The foregoing and other features and advantages of this disclosure will become more apparent from the following detailed description of several implementations which proceeds with reference to the accompanying figures.

### BRIEF DESCRIPTION OF THE FIGURES

FIGS. 1A-1E. Reduced net positive charge of VRC07-523-03FR3 variants correlated with their reduced affinity to heparin and reduced polyreactivity. (A) Closeup view of 03FR3 loop insertion into VRC01.23LS heavy chain. Four Asp's were highlighted in red. (B) Insertion of 03FR3 loop into VRC07-523LS reduced HEp-2 cell binding. Antibodies scored greater than 1 at 25 μg/ml were considered polyreactive. (C) Sequence alignments of VRC07-523LS variants (D) Polyreactivity of VRC07-523LS variants assessed in HEp-2 cell bindings (left panel). VRC07-523LS variants on heparin affinity chromatography (right panel). Redlines represent NaCl gradient. Retention volumes of VRC07-523LS variants on heparin chromatography correlated with polyreactivity. (E) Correlations between heparin retention volume vs net positive charge, HEp-2 cell bindings vs isoelectric point (pI), HEp-2 cell bindings vs. net positive charge of VRC07-523LS variants.

FIG. 2. Workflow of improving PK for HIV antibodies by surface charge alteration. 1) Selection of Arg or Lys residues in the variable region based on their accessible surface areas. 2) Screening the residues that contact with epitope and located within 5A from the epitope. 3) Generation of a panel of antibody variants 4) Assessing neutralization potency and breadth, polyreactivity, and retention volumes on heparin affinity chromatography and down selection of antibodies for PK studies. 6) Assessment of pharmacokinetics in human FcRn knock-in mouse model.

FIGS. 3A-3F. VRC07-523LS variants with select Arg or Lys to Asp, Glu, or Ser substitutions showed improved pharmacokinetics. (A) Arg and Lys residues in the variable region of VRC07-523 selected for Asp, Glu, or Ser substitution were demonstrated on the VRC07-523 Fab modeled using the VRC01.23-bound BG505 DS-SOSIP Env trimer (PDB ID:6VI0). (B) VRC07-523LS charge variants were plotted according to the fold change of geometric mean IC80 values and the retention volumes on heparin column chromatography. (C) Pharmacokinetics of VRC07-523LS variants in human FcRn knock-in mice after administering a dose of 5 mg/kg intravenously. (D) PK properties, affinity to heparin, geometric mean IC80 fold changes against a 12-isolate panel, and net charge of VRC07-523LS variants. (E, F) Neutralizing activity of VRC07-523LS variants against a 208-virus panel.

FIGS. 4A-4G. N6LS variants with select Arg or Lys to Asp, Gln, or Glu mutations showed improved pharmacokinetics. (A, B) Arg and Lys residues in the variable region of N6LS selected for Asp, Gln, or Glu substitution were shown in surface representation to the N6LS Fab modeled using VRC01.23-bound BG505 DS-SOSIP Env trimer (PDB ID:6VI0). (C) N6LS charge variants were plotted according to the geometric mean IC80 fold change and the retention volumes on heparin column chromatography. (D) Pharmacokinetics of N6LS variants in human FcRn knock-in mice after injecting a dose of 5 mg/kg intravenous. (E) PK properties, affinity to heparin, IC80 fold changes against a 12-isolate panel, and net charge of N6LS variants. (F, G) Neutralizing potency against a 208-virus panel.

FIGS. 5A-5D. Binding affinities of VRC07-523LS variants to heparin correlated with their affinities to FcRn at pH 7.4 when the Fab regions were free to engage with matrices of biosensors. (A) FcRn/β2m was passed over antibody variants captured by gp120 core immobilized on a CM5 chip. (B) Ni-NTA biosensors coated with FcRn/β2m were dipped into wells containing VRC07-523LS variants (C) The plot of binding affinity to FcRn/β2m vs. the retention volume of harpin chromatography of (A). (D) The plot of binding affinity to FcRn/β2m vs. the retention volume of harpin chromatography of (B).

FIG. 6A-6C. Arg or Lys is highly prevalent among amino acids at select positions in the variable region of human antibodies. (A) The relative frequency of amino acid in the variable region of human antibodies where Arg or Lys is the most or highly prevalent. (B) Accessible surface areas were calculated from the structures of eight HIV-1 antibodies. Arg/Lys residues with high ASA and high relative frequency were highlighted in gray background. (C) Arg/Lys residues highly prevalent in human antibodies were shown in stick representation. Close-up view of four Arg/Lys that interact with neighboring Asp via salt bridges.

FIG. 7A-7D. Estimation of half-life in humans using human FcRn mice data. (A) Linear Regression of LS-variant antibodies with 100,000 bootstrap resamples used to estimate 95% c.i. (r2=0.872) was used to estimate half-life in humans and parameter β in two-compartment models. (B) Scaled models of charge N6LS variants (C) Scaled models of VRC07-523LS charge variants. (D) Table summarizing model parameters and concentration values at 180 and 360 days for the charge variants relative to the parent antibody.

FIG. 8. Substitution of Arg with Asp reduced polyreactivity of VRC07-523LS and N6LS variants. HEp-2 cell staining assay against VRC01 variants was performed in the concentration of g/ml along with control antibodies, VRC01-LS, 4E10, VRC07-523LS, and VRC07-G54W. Control antibodies were assigned a score between 0 and 3. Test antibodies scored greater than 1 at 25 μg/ml were considered polyreactive.

FIGS. 9A-9B. Locations where Arg or Lys is most or highly prevalent are relatively conserved in the VH and VL region of human antibodies. (A) The VH region of human antibodies in Kabat numbering where Arg or Lys is the most or highly prevalent. (B) The VL region of human antibodies in Kabat numbering where Arg or Lys is the most or highly prevalent. The relative frequency of Arg and Lys is shown.

FIGS. 10A-10D. VRC07-523LS and N6LS charge variants showed their PK parameters significantly improved compared to their parentals. (A) Improved PK parameters-half-life, area under the curve of serum mAb concentration vs time, and clearance—of VRC07-523LS charge variants. (B) Improved PK parameters-half-life, area under the curve of serum mAb concentration vs time, and clearance—of N6LS charge variants. (A and B) P value was calculated using unpaired t test with the 95% confidence interval. (C) Correlation between heparin column retention volume and half-life. (D) Affinity to heparin vs. geometric mean IC80 fold change of VRC07-523LS variants.

FIG. 11 shows a table of anti-Cardiolipin ELISA results.

FIGS. 12A-12C shows a table showing neutralization breadth and potency of VRC07-523LS and N6LS variants on a 12-viruse panel.

FIGS. 13A and 13B show tables of neutralization potency and heparin chromatography of (A) VRC07-523LS variants and (B) N6LS variants.

FIG. 14 show a table providing comparison of protein expression between VRC07-523LS variants with light chain R24D and R24E mutation.

FIG. 15. Arginines in the Variable domain of 10E8VLS and 10E8v4-5RLS were replaced with Asp or Glu.

FIG. 16. Lysines in the Variable domain of 10E8VLS and 10E8v4-5RLS were replaced with Asp or Glu.

FIG. 17A-17C. 10E8VLS and 10E8v4-5RLS Variants: Affinity to Heparin and Neutralization Potency.

FIG. 18. 10E8VLS.C1 and .C2 Variants Showed Improved PK.

FIG. 19. Affinity to Heparin vs. Neutralization Potency for 10E8VLS variants.

FIG. 20. Affinity to Heparin and Neutralization Potency of 10E8v4-5R Variants.

FIG. 21. 10E8v4-5RLS.C13, C14, and C15 Variants Showed Improved PK.

FIG. 22. 10E8v4-5R.C27 and C30 Variants Showed Improved PK with the Potency Comparable to 10E8v4-5R_LS.

FIG. 23. 10E8v4.cc11 (a constant domain charge variant) showed Improved PK with the Potency Comparable to 10E8v4-5R_LS.

FIG. 24A-24B. Summary of Results for antibody charge variants.

FIG. 25. VRC07-523LS.cv1 (mutations in the constant domain) and .cv34 (mutations in the constant and variable domains) showed Improved PK while maintaining potency.

FIG. 26. N6LS.cv1 (mutations in the constant domain) and .cv49 (mutations in the constant and the variable domains) showed Improved PK while maintaining potency.

FIG. 27. VRC01.23LS.cv1 (mutations in the constant domain) and .cv34 (mutations in the constant and the variable domains) showed Improved PK while maintaining potency.

FIG. 28. 10E8v4-5R.cv1 (mutations in the constant domain) and .cv30 (mutations in the constant and the variable domains) showed Improved PK while maintaining potency.

FIG. 29. Schematic diagram showing the format of antibodies modified with addition of the acidic tail of alpha-synuclein (ATS(a) to the heavy or light chain, or both.

FIG. 30. VRC01.23LS.ATS1, VRC01.23LS.ATS4, and VRC01.23LS.ATS5 showed improved PK while maintaining or improving potency.

FIG. 31. VRC07-523LS.ATS1, VRC01.23LS.ATS4, and VRC07-523LS.ATS10 showed improved PK while maintaining or improving potency.

FIG. 32. N6LS.ATS4, N6LSLS.ATS8, and N6LS.ATS9 showed Improved PK while maintaining or improving potency.

FIG. 33. 10E8.ATS4 and 10E8.ATS11 showed improved PK while maintaining or improving potency. 10E8.ATS13 showed PK comparable to 10E8v4-5RLS while it is about 5-fold more potent than 10E8v4-5RLS.

### DETAILED DESCRIPTION

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various implementations, the following explanations of terms are provided: Administration: The introduction of a composition into a subject by a chosen route.

Administration can be local or systemic. For example, if the chosen route is intravenous, the composition is administered by introducing the composition into a vein of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Antibody and Antigen Binding Fragment: An immunoglobulin, antigen-binding fragment, or derivative thereof, that specifically binds and recognizes an analyte (antigen) such as HIV-1 Env. The term “antibody” is used herein in the broadest sense and encompasses various antibody structures, including but not limited to monoclonal antibodies, polyclonal antibodies, multispecific antibodies (e.g., bispecific and trispecific antibodies), and antigen-binding fragment, so long as they exhibit the desired antigen-binding activity.

Non-limiting examples of antibodies include, for example, intact immunoglobulins and variants and fragments thereof known in the art that retain binding affinity for the antigen. Examples of antigen-binding fragment include but are not limited to Fv, Fab, Fab′, Fab′-SH, F(ab′)₂; diabodies; linear antibodies; single-chain antibody molecules (e.g. scFv); and multispecific antibodies formed from antibody fragments. Antigen-binding fragments include those produced by the modification of whole antibodies and those synthesized de novo using recombinant DNA methodologies (see, e.g., Kontermann and Dubel (Eds.), Antibody Engineering, Vols. 1-2, 2^{nd} ed., Springer-Verlag, 2010).

Antibodies also include genetically engineered forms such as chimeric antibodies (such as humanized murine antibodies) and heteroconjugate antibodies (such as bispecific antibodies).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (k) and kappa (x). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain). In combination, the heavy and the light chain variable regions specifically bind the antigen.

References to “V_{H}” or “VH” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, 5^{th} ed., NIH Publication No. 91-3242, Public Health Service, National Institutes of Health, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, 5^{th} ed., NIH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273(4):927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27(1):55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L}CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

In some implementations, a disclosed antibody includes a heterologous constant domain. For example the antibody includes a constant domain that is different from a native constant domain, such as a constant domain including one or more modifications (such as the “LS” mutations) to increase half-life.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions. (See, for example, Greenfield (Ed.), Antibodies: A Laboratory Manual, 2^{nd} ed. New York: Cold Spring Harbor Laboratory Press, 2014.) A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one implementation, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some implementations, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} ed. New York: Cold Spring Harbor Laboratory Press, 2004.; Lonberg, Nat. Biotechnol., 23(9): 1117-1125, 2005; Lonberg, Curr. Opin. Immunol., 20(4):450-459, 2008).

A “bispecific antibody” is a recombinant molecule composed of two different antigen binding domains that consequently binds to two different antigenic epitopes. Bispecific antibodies include chemically or genetically linked molecules of two antigen-binding domains. The antigen binding domains can be linked using a linker. The antigen binding domains can be monoclonal antibodies, antigen-binding fragments (e.g., Fab, scFv), or combinations thereof. A bispecific antibody can include one or more constant domains, but does not necessarily include a constant domain.

A “parent” antibody is an antibody that is used as a reference or comparison when referring to another antibody that is not the parent antibody. For example, a test antibody that has the same CDRs as a particular parent antibody has CDRs that are identical to the CDRs of the parent antibody, but the remainder of the test antibody could be different from the parent antibody.

Antibody or antigen binding fragment that neutralizes HIV-1: An antibody or antigen binding fragment that specifically binds to HIV-1 Env (for example, that binds gp120) in such a way as to inhibit a biological function associated with HIV-1 Env (such as binding to its target receptor). In several implementations, an antibody or antigen binding fragment that neutralizes HIV-1 reduces the infectious titer of HIV-1.

Broadly neutralizing antibodies to HIV-1 are distinct from other antibodies to HIV-1 in that they neutralize a high percentage of the many types of HIV-1 in circulation. In some implementations, broadly neutralizing antibodies to HIV-1 are distinct from other antibodies to HIV-1 in that they neutralize a high percentage (such as at least 80% or at least 90%) of the many types of HIV-1 in circulation. Non-limiting examples of HIV-1 broadly neutralizing antibodies include N6, VRC07-523, and 10E8.

Biological sample: A sample obtained from a subject. Biological samples include all clinical samples useful for detection of disease or infection (for example, HIV-1 infection) in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having or suspected of having an HIV-1 infection.

CD4: Cluster of differentiation factor 4 polypeptide; a T-cell surface protein that mediates interaction with the MHC class II molecule. CD4 also serves as the primary receptor site for HIV-1 on T-cells during HIV-1 infection. CD4 is known to bind to gp120 from HIV-1. The known sequence of the CD4 precursor has a hydrophobic signal peptide, an extracellular region of approximately 370 amino acids, a highly hydrophobic stretch with significant identity to the membrane-spanning domain of the class II MHC beta chain, and a highly charged intracellular sequence of 40 resides (Maddon, Cell 42:93, 1985).

Conditions sufficient to form an immune complex: Conditions which allow an antibody or antigen binding fragment to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Greenfield (Ed.), Antibodies: A Laboratory Manual, 2^{nd} ed. New York: Cold Spring Harbor Laboratory Press, 2014, for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The formation of an immune complex can be detected through conventional methods, for instance immunohistochemistry (IHC), immunoprecipitation (IP), flow cytometry, immunofluorescence microscopy, ELISA, immunoblotting (for example, Western blot), magnetic resonance imaging (MRI), computed tomography (CT) scans, radiography, and affinity chromatography. Immunological binding properties of selected antibodies may be quantified using known methods.

Conjugate: A complex of two molecules linked together, for example, linked together by a covalent bond. In one implementation, an antibody is linked to an effector molecule; for example, an antibody that specifically binds to HIV-1 Env covalently linked to an effector molecule. The linkage can be by chemical or recombinant means. In one implementation, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

Conservative amino acid substitution: “Conservative” amino acid substitutions are those substitutions that do not substantially affect a function of a protein, such as the ability of the protein to interact with a target protein.

In some implementations, a conservative amino acid substitution in an HIV Env-specific antibody is one that does not reduce binding of the antibody to HIV Env by more than 10% (such as by more than 5%) compared to the HIV Env binding of the corresponding antibody lacking the conservative amino acid substitution. In some implementations, the HIV Env-specific antibody includes no more than 10 (such as no more than 5, no more than 3, no more than 2, or no more than 1) conservative substitutions compared to a reference antibody and retain specific binding activity for HIV Env, and/or HIV-1 neutralization activity.

Typically, individual substitutions, deletions or additions which alter, add or delete a single amino acid or a small percentage of amino acids (for instance less than 5%, in some implementations less than 1%) in an encoded sequence are conservative variations where the alterations result in the substitution of an amino acid with a chemically similar amino acid. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some implementations, the control is a negative control, such as sample obtained from a healthy patient not infected with HIV-1. In other implementations, the control is a positive control, such as a tissue sample obtained from a patient diagnosed with HIV-1 infection. In still other implementations, the control is a historical control or standard reference value or range of values (such as a previously tested control sample, such as a group of HIV-1 patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Detectable marker: A detectable molecule (also known as a label) that is conjugated directly or indirectly to a second molecule, such as an antibody, to facilitate detection of the second molecule. For example, the detectable marker can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). Methods for using detectable markers and guidance in the choice of detectable markers appropriate for various purposes are discussed for example in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Detecting: To identify the existence, presence, or fact of something.

Effector molecule: A molecule intended to have or produce a desired effect; for example, a desired effect on a cell to which the effector molecule is targeted. Effector molecules can include, for example, polypeptides and small molecules. In one non-limiting example, the effector molecule is a toxin. Some effector molecules may have or produce more than one desired effect.

Epitope: An antigenic determinant. These are particular chemical groups or peptide sequences on a molecule that are antigenic, i.e. that elicit a specific immune response. An antibody specifically binds a particular antigenic epitope on a polypeptide. In some examples a disclosed antibody specifically binds to an epitope on gp120.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence.

Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) in front of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

HIV-1 Envelope protein (Env): The HIV-1 envelope protein is initially synthesized as a precursor protein of 845-870 amino acids in size, designated gp160. Individual gp160 polypeptides form a homotrimer and undergo glycosylation within the Golgi apparatus as well as processing to remove the signal peptide, and cleavage by a cellular protease between approximately positions 511/512 to generate separate gp120 and gp41 polypeptide chains, which remain associated as gp120/gp41 protomers within the homotrimer. The ectodomain (that is, the extracellular portion) of the HIV-1 Env trimer undergoes several structural rearrangements from a prefusion mature (cleaved) closed conformation that evades antibody recognition, through intermediate conformations that bind to receptors CD4 and co-receptor (either CCR5 or CXCR4), to a postfusion conformation.

The numbering used in the disclosed HIV-1 Env proteins and fragments thereof is relative to the HXB2 numbering scheme as set forth in Numbering Positions in HIV Relative to HXB2CG Bette Korber et al., Human Retroviruses and AIDS 1998: A Compilation and Analysis of Nucleic Acid and Amino Acid Sequences. Korber et al., Eds. Theoretical Biology and Biophysics Group, Los Alamos National Laboratory, Los Alamos, NM, which is incorporated by reference herein in its entirety.

HIV-1 gp120: A polypeptide that is part of the HIV-1 Env protein. Mature gp120 includes approximately HIV-1 Env residues 31-511, contains most of the external, surface-exposed, domains of the HIV-1 Env trimer, and it is gp120 which binds both to cellular CD4 receptors and to cellular chemokine receptors (such as CCR5). A mature gp120 polypeptide is an extracellular polypeptide that interacts with the gp41 ectodomain to form an HIV-1 Env protomer that trimerizes to form the HIV-1 Env trimer.

HIV-1 gp41: A polypeptide that is part of the HIV-1 Env protein. Mature gp41 includes approximately HIV-1 Env residues 512-860, and includes cytosolic-, transmembrane-, and ecto-domains. The gp41 ectodomain (including approximately HIV-1 Env residues 512-644) can interact with gp120 to form an HIV-1 Env protomer that trimerizes to form the HIV-1 Env trimer. Human Immunodeficiency Virus type 1 (HIV-1): A retrovirus that causes immunosuppression in humans (HIV-1 disease), and leads to a disease complex known as the acquired immunodeficiency syndrome (AIDS). “HIV-1 disease” refers to a well-recognized constellation of signs and symptoms (including the development of opportunistic infections) in persons who are infected by an HIV-1 virus, as determined by antibody or western blot studies. Laboratory findings associated with this disease include a progressive decline in T cells. Related viruses that are used as animal models include simian immunodeficiency virus (SIV) and feline immunodeficiency virus (FIV). Treatment of HIV-1 with HAART has been effective in reducing the viral burden and ameliorating the effects of HIV-1 infection in infected individuals.

HXB2 numbering system: A reference numbering system for HIV-1 protein and nucleic acid sequences, using HIV-1 HXB2 strain sequences as a reference for all other HIV-1 strain sequences. The person of ordinary skill in the art is familiar with the HXB2 numbering system, and this system is set forth in “Numbering Positions in HIV Relative to HXB2CG,” Bette Korber et al., Human Retroviruses and AIDS 1998: A Compilation and Analysis of Nucleic Acid and Amino Acid Sequences. Korber B, Kuiken C L, Foley B, Hahn B, McCutchan F, Mellors J W, and Sodroski J, Eds. Theoretical Biology and Biophysics Group, Los Alamos National Laboratory, Los Alamos, NM, which is incorporated by reference herein in its entirety. HXB2 is also known as: HXBc2, for HXB clone 2; HXB2R, in the Los Alamos HIV database, with the R for revised, as it was slightly revised relative to the original HXB2 sequence; and HXB2CG in GENBANK™, for HXB2 complete genome. The numbering used in gp120 polypeptides disclosed herein is relative to the HXB2 numbering scheme. For reference, the amino acid sequence of HIV-1 Env of HXB2 is set forth below:

MRVKEKYQHLWRWGWRWGTMLLGMLMICSATEKLWVTVYYGVPVWKEATT
TLFCASDAKAYDTEVHNVWATHACVPTDPNPQEVVLVNVTENFNMWKNDM
VEQMHEDIISLWDQSLKPCVKLTPLCVSLKCTDLKNDTNTNSSSGRMIME
KGEIKNCSFNISTSIRGKVQKEYAFFYKLDIIPIDNDTTSYKLTSCNTSV
ITQACPKVSFEPIPIHYCAPAGFAILKCNNKTFNGTGPCTNVSTVQCTHG
IRPVVSTQLLINGSLAEEEVVIRSVNFTDNAKTIIVQLNTSVEINCTRPN
NNTRKRIRIQRGPGRAFVTIGKIGNMRQAHCNISRAKWNNTLKQIASKLR
EQFGNNKTIIFKQSSGGDPEIVTHSFNCGGEFFYCNSTQLFNSTWFNSTW
STEGSNNTEGSDTITLPCRIKQIINMWQKVGKAMYAPPISGQIRCSSNIT
GLLLTRDGGNSNNESEIFRPGGGDMRDNWRSELYKYKVVKIEPLGVAPTK
AKRRVVQREKRAVGIGALFLGFLGAAGSTMGAASMTLTVQARQLLSGIVQ
QQNNLLRAIEAQQHLLQLTVWGIKQLQARILAVERYLKDQQLLGIWGCSG
KLICTTAVPWNASWSNKSLEQIWNHITWMEWDREINNYTSLIHSLIEESQ
NQQEKNEQELLELDKWASLWNWFNITNWLWYIKLFIMIVGGLVGLRIVFA
VLSIVNRVRQGYSPLSFQTHLPTPRGPDRPEGIEEEGGERDRDRSIRLVN
GSLALIWDDLRSLCLFSYHRLRDLLLIVTRIVELLGRRGWEALKYWWNLL
QYWSQELKNSAVSLLNATAIAVAEGTDRVIEVVQGACRAIRHIPRRIRQG
LERILL
(GENBANK® Accession No. K03455, SEQ ID NO: 100).

IgA: A polypeptide belonging to the class of antibodies that are substantially encoded by a recognized immunoglobulin alpha gene. In humans, this class or isotype comprises IgA1 and IgA2. IgA antibodies can exist as monomers, polymers (referred to as pIgA) of predominantly dimeric form, and secretory IgA. The constant chain of wild-type IgA contains an 18-amino-acid extension at its C-terminus called the tail piece (tp). Polymeric IgA is secreted by plasma cells with a 15-kDa peptide called the J chain linking two monomers of IgA through the conserved cysteine residue in the tail piece.

IgG: A polypeptide belonging to the class or isotype of antibodies that are substantially encoded by a recognized immunoglobulin gamma gene. In humans, this class comprises IgG1, IgG2, IgG3, and IgG4.

Immune complex: The binding of antibody or antigen binding fragment (such as a scFv) to a soluble antigen forms an immune complex. The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, radiography, and affinity chromatography.

Inhibiting a disease or condition: Reducing the full development of a disease or condition in a subject, for example, reducing the development of AIDS in a subject infected with HIV-1 or reducing symptoms associated with the HIV-1 infection. This includes neutralizing, antagonizing, prohibiting, preventing, restraining, slowing, disrupting, stopping, or reversing progression or severity of the disease or condition.

Inhibiting a disease or condition includes a prophylactic intervention administered before the disease or condition has begun to develop (for example a treatment initiated in a subject at risk of an HIV-1 infection, but not infected by HIV-1) that reduces subsequent development of the disease or condition and also to amelioration of one or more signs or symptoms of the disease or condition following development. Additionally, inhibiting a disease or condition includes a therapeutic intervention administered after a disease or condition has begun to develop (for example, a treatment administered following diagnosis of a subject with HIV-1 infection) that ameliorates one or more signs or symptoms of the disease or condition in the subject. The term “ameliorating,” with reference to inhibiting a disease or condition refers to any observable beneficial effect of the intervention intended to inhibit the disease or condition. The beneficial effect can be evidenced, for example, by a delayed onset of clinical symptoms of the disease or condition in a susceptible subject, a reduction in severity of some or all clinical symptoms of the disease or condition, a slower progression of the disease or condition, an improvement in the overall health or well-being of the subject, a reduction in infection, or by other parameters that are specific to the particular disease or condition.

Isolated: A biological component (such as a nucleic acid, peptide, protein or protein complex, for example an antibody) that has been substantially separated, produced apart from, or purified away from other biological components in the cell of the organism in which the component naturally occurs, that is, other chromosomal and extra-chromosomal DNA and RNA, and proteins. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

Kabat position: A position of a residue in an amino acid sequence that follows the numbering convention delineated by Kabat et al. (Sequences of Proteins of Immunological Interest, 5^{th} ed., NIH Publication No. 91-3242, Public Health Service, National Institutes of Health, U.S.

### Department of Health and Human Services, 1991).

Linker: A bi-functional molecule that can be used to link two molecules into one contiguous molecule, for example, to link an effector molecule to an antibody. Non-limiting examples of peptide linkers include glycine-serine linkers.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an scFv. The linkage can be either by chemical or recombinant means. “Chemical means” refers to a reaction between the antibody moiety and the effector molecule such that there is a covalent bond formed between the two molecules to form one molecule.

Nucleic acid (molecule or sequence): A deoxyribonucleotide or ribonucleotide polymer or combination thereof including without limitation, cDNA, mRNA, genomic DNA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides.

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and the non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed agents.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some implementations, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. A polypeptide includes both naturally occurring proteins, as well as those that are recombinantly or synthetically produced. A polypeptide has an amino terminal (N-terminal) end and a carboxy-terminal (C-terminal) end. In some implementations, the polypeptide is a disclosed antibody or a fragment thereof.

Purified: The term purified does not require absolute purity; rather, it is intended as a relative term. Thus, for example, a purified peptide preparation is one in which the peptide or protein (such as an antibody) is more enriched than the peptide or protein is in its natural environment within a cell. In one implementation, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several implementations, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Sequence identity: The identity between two or more nucleic acid sequences, or two or more amino acid sequences, is expressed in terms of the identity between the sequences. Sequence identity can be measured in terms of percentage identity; the higher the percentage, the more identical the sequences. Homologs and variants of a V_{L} or a V_{H} of an antibody that specifically binds a target antigen are typically characterized by possession of at least about 75% sequence identity, for example at least about 80%, 85%, 90%, 91%, 92%, 93%, 94%, 95%, 96%, 97%, 98% or 99% sequence identity counted over the full-length alignment with the amino acid sequence of interest.

Methods of alignment of sequences for comparison are well known in the art. Various programs and alignment algorithms are described in: Smith and Waterman, Adv. Appl. Math. 2(4):482-489, 1981; Needleman and Wunsch, J. Mol. Biol. 48(3):443-453, 1970; Pearson and Lipman, Proc. Natl. Acad. Sci. U.S.A. 85(8):2444-2448, 1988; Higgins and Sharp, Gene, 73(1):237-244, 1988; Higgins and Sharp, Bioinformatics, 5(2):151-3, 1989; Corpet, Nucleic Acids Res. 16(22):10881-10890, 1988; Huang et al. Bioinformatics, 8(2):155-165, 1992; and Pearson, Methods Mol. Biol. 24:307-331, 1994. Altschul et al., J. Mol. Biol. 215(3):403-410, 1990, presents a detailed consideration of sequence alignment methods and homology calculations. The NCBI Basic Local Alignment Search Tool (BLAST) (Altschul et al., J. Mol. Biol. 215(3):403-410, 1990) is available from several sources, including the National Center for Biological Information and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Generally, once two sequences are aligned, the number of matches is determined by counting the number of positions where an identical nucleotide or amino acid residue is present in both sequences. The percent sequence identity between the two sequences is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

Specifically bind: When referring to an antibody or antigen binding fragment, refers to a binding reaction which determines the presence of a target protein in the presence of a heterogeneous population of proteins and other biologics. Thus, under designated conditions, an antibody binds preferentially to a particular target protein, peptide or polysaccharide (such as an antigen present on the surface of a pathogen, for example HIV-1 Env) and does not bind in a significant amount to other proteins present in the sample or subject. A limited degree of non-specific interaction may occur between an antibody (such as an antibody that specifically binds to HIV-1 Env) and a non-target (such as a cell that does not express HIV-1 Env). Specific binding can be determined by methods known in the art. See Greenfield (Ed.), Antibodies: A Laboratory Manual, 2^{nd} ed. New York: Cold Spring Harbor Laboratory Press, 2014, for a description of immunoassay formats and conditions that can be used to determine specific immunoreactivity.

With reference to an antibody-antigen complex, specific binding of the antigen and antibody has a K_{D} of less than about 10⁻⁷ Molar, such as less than about 10⁻⁸ Molar, 10⁻⁹, or even less than about 10⁻¹⁰ Molar. K_{D} refers to the dissociation constant for a given interaction, such as a polypeptide-ligand interaction or an antibody-antigen interaction. For example, for the bimolecular interaction of an antibody or antigen binding fragment and an antigen it is the concentration of the individual components of the bimolecular interaction divided by the concentration of the complex.

Subject: Living multicellular vertebrate organisms, a category that includes human and non-human mammals. In an example, a subject is a human. In a particular example, the subject is a newborn infant. In an additional example, a subject is selected that is in need of inhibiting an HIV-1 infection. For example, the subject is uninfected and at risk of HIV-1 infection.

Therapeutically effective amount: A quantity of a specific substance sufficient to achieve a desired effect in a subject being treated. For instance, this can be the amount necessary to prevent, treat (including prophylaxis), reduce and/or ameliorate the symptoms or underlying causes of a disorder or disease, such as HIV-1 infection. In some implementations, a therapeutically effective amount is sufficient to reduce or eliminate a symptom of HIV-1 infection, such as AIDS. For instance, this can be the amount necessary to inhibit or prevent HIV-1 replication or to measurably alter outward symptoms of the HIV-1 infection. Ideally, a therapeutically effective amount provides a therapeutic effect without causing a substantial cytotoxic effect in the subject.

In some implementations, administration of a therapeutically effective amount of a disclosed antibody or antigen binding fragment that binds to HIV-1 Env can reduce or inhibit an HIV-1 infection (for example, as measured by infection of cells, or by number or percentage of subjects infected by HIV-1, or by an increase in the survival time of infected subjects) by a desired amount, for example by at least 10%, at least 20%, at least 50%, at least 60%, at least 70%, at least 80%, at least 90%, at least 95%, at least 98%, or even at least 100% (elimination or prevention of detectable HIV-1 infection), as compared to a suitable control.

A therapeutically effective amount of an antibody or antigen binding fragment that specifically binds gp120 that is administered to a subject will vary depending upon a number of factors associated with that subject, for example the overall health and/or weight of the subject. A therapeutically effective amount can be determined by varying the dosage and measuring the resulting therapeutic response, such as, for example, a reduction in viral titer. Therapeutically effective amounts also can be determined through various in vitro, in vivo or in situ immunoassays.

A therapeutically effective amount encompasses a fractional dose that contributes in combination with previous or subsequent administrations to attaining a therapeutic response. For example, a therapeutically effective amount of an agent can be administered in a single dose, or in several doses, for example daily, during a course of treatment lasting several days or weeks. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Transformed: A transformed cell is a cell into which a nucleic acid molecule has been introduced by molecular biology techniques. As used herein, the term transformed and the like (e.g., transformation, transfection, transduction, etc.) encompasses all techniques by which a nucleic acid molecule might be introduced into such a cell, including transduction with viral vectors, transformation with plasmid vectors, and introduction of DNA by electroporation, lipofection, and particle gun acceleration.

Vector: An entity containing a nucleic acid molecule (such as a DNA or RNA molecule) bearing a promoter(s) that is operationally linked to the coding sequence of a protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses. In some implementations, a viral vector comprises a nucleic acid molecule encoding a disclosed antibody or antigen binding fragment that specifically binds to HIV-1 gp120 and neutralizes HIV-1. In some implementations, the viral vector can be an adeno-associated virus (AAV) viral vector.

### II. Description of Several Implementations

Isolated monoclonal antibodies and antigen binding fragments that specifically bind to HIV-1 Env are provided. In several implementations, the antibodies and antigen binding fragments are used to neutralize HIV-1. Also disclosed herein are compositions including the antibodies and antigen binding fragments and a pharmaceutically acceptable carrier. Nucleic acids encoding the antibodies or antigen binding fragments, expression vectors (such as adeno-associated virus (AAV) viral vectors) including these nucleic acids are also provided.

The antibodies, antigen binding fragments, nucleic acid molecules, and compositions can be used for research, diagnostic and therapeutic purposes. For example, the monoclonal antibodies and antigen binding fragments can be used to diagnose or treat a subject with an HIV-1 infection, or can be administered prophylactically to prevent HIV-1 infection in a subject. In some implementations, the antibodies can be used to determine HIV-1 titer in a subject.

### A. Antibodies and Antigen Binding Fragments

In some implementations, a monoclonal antibody or antigen binding fragment thereof is provided comprising a heavy chain variable region (V_{H}) and a light chain variable region (V_{L}) comprising amino acid sequences respectively set forth as SEQ ID NOs: 1 and 5, respectively (VRC07-523LS.v11); SEQ ID NOs: 7 and 5, respectively (VRC07-523LS.v14); EQ ID NOs: 9 and 10, respectively (VRC07-523LS.v21); EQ ID NOs: 13 and 14, respectively (VRC07-523LS.v26); SEQ ID NOs: 17 and 14, respectively (VRC07-523LS.v32); SEQ ID NOs: 17 and 10, respectively (VRC07-523LS.v34); SEQ ID NOs: 59 and 10, respectively (VRC07-523LS.cv34); SEQ ID NOs: 23 and 24, respectively (N6LS.C1); SEQ ID NOs: 19 and 27, respectively (N6LS.15); SEQ ID NOs: 29 and 30, respectively (N6LS.30); SEQ ID NOs: 29 and 27, respectively (N6LS.35); SEQ ID NOs: 19 and 33, respectively (N6LS.47); SEQ ID NOs: 35 and 33, respectively (N6LS.49); SEQ ID NOs: 37 and 33, respectively (N6LS.51); SEQ ID NOs: 39 and 33, respectively (N6LS.58); SEQ ID NO: 23 and 33, respectively (N6LS.ATS8); SEQ ID NOs: 45 and 42, respectively (10E8v4-5RLS.C12); SEQ ID NOs: 45 and 47, respectively (10E8v4-5RLS.C13); SEQ ID NOs: 45 and 49, respectively (10E8v4-5RLS.C14); SEQ ID NOs: 51 and 52, respectively (10E8v4-5RLS.C15); SEQ ID NOs: 53 and 42, respectively (10E8v4-5RLS.C18); SEQ ID NOs: 55 and 42, respectively (10E8v4-5RLS.C24); SEQ ID NO: 65 and 66, respectively (10E8v4-5RLS.C27); SEQ ID NO: 65 and 69, respectively (10E8v4-5RLS.C30); SEQ ID NO: 98 and 66, respectively (10E8.ATS13); SEQ ID NO: 77 and 2, respectively (VRC01.23LS.cv1); SEQ ID NO: 59 and 10, respectively (VRC01.23LS.cv34); or SEQ ID NO: 77 and 10, respectively (VRC01.23LS.ATS5). The monoclonal antibody or antigen binding fragment specifically binds to HIV-1 Env and neutralizes HIV-1. In several implementations, the monoclonal antibody or antigen binding fragment specifically binds to HIV-1 Env, neutralizes HIV-1 Env, is not autoreactive, and has improved in vivo half-life.

In some implementations, a monoclonal antibody is provided comprising a heavy chain and a light chain comprising amino acid sequences set forth as SEQ ID NOs: 3 and 6, respectively (VRC07-523LS.v11); SEQ ID NOs: 8 and 6, respectively (VRC07-523LS.v14); SEQ ID NOs: 11 and 12, respectively (VRC07-523LS.v21); SEQ ID NOs: 15 and 16, respectively (VRC07-523LS.v26); SEQ ID NOs: 18 and 16, respectively (VRC07-523LS.v32); SEQ ID NOs: 18 and 12, respectively (VRC07-523LS.v34); SEQ ID NOs: 60 and 61, respectively (VRC07-523LS.cv34); SEQ ID NOs: 25 and 26, respectively (N6LS.C1); SEQ ID NOs: 21 and 28, respectively (N6LS.15); SEQ ID NOs: 31 and 32, respectively (N6LS.30); SEQ ID NOs: 31 and 28, respectively (N6LS.35); SEQ ID NOs: 21 and 34, respectively (N6LS.47); SEQ ID NOs: 36 and 34, respectively (N6LS.49); SEQ ID NO: 64 and 97, respectively (N6LS.cv49); SEQ ID NOs: 38 and 34, respectively (N6LS.51); SEQ ID NOs: 40 and 34, respectively (N6LS.58); SEQ ID NO: 86 and 87, respectively (N6LS.ATS8); SEQ ID NOs: 46 and 44, respectively (10E8v4-5RLS.C12); SEQ ID NOs: 46 and 48, respectively (10E8v4-5RLS.C13); SEQ ID NOs: 46 and 50, respectively (10E8v4-5RLS.C14); SEQ ID NOs: 52 and 44, respectively (10E8v4-5RLS.C15); SEQ ID NOs: 53 and 44, respectively (10E8v4-5RLS.C18); SEQ ID NOs: 56 and 44, respectively (10E8v4-5RLS.C24); SEQ ID NO: 67 and 68, respectively (10E8v4-5RLS.C27); SEQ ID NO: 67 and 70, respectively (10E8v4-5RLS.C30); SEQ ID NO: 75 and 76, respectively (10E8v4-5RLS.cv30); SEQ ID NO: 94 and 93, respectively (10E8.ATS13); SEQ ID NO: 78 and 58, respectively (VRC01.23LS.cvl); SEQ ID NO: 60 and 61, respectively (VRC01.23LS.cv34); SEQ ID NO: 96 and 83, respectively (VRC01.23LS.ATS5); SEQ ID NOs: 57 and 58, respectively (VRC07-523LS.cv1); SEQ ID NOs: 62 and 63, respectively (N6LS.cv1); SEQ ID NO: 43 and 71, respectively (10E8v4.cc11); SEQ ID NO: 72 and 44, respectively (10E8v4.cc16); or SEQ ID NO: 73 and 74, respectively (10E8v4-5RLS.cvl). The monoclonal antibody specifically binds to HIV-1 Env and neutralizes HIV-1. In several implementations, the monoclonal antibody specifically binds to HIV-1 Env, neutralizes HIV-1 Env, is not autoreactive, and has improved in vivo half-life.

In several aspects, the monoclonal antibody further comprises an alpha-synuclein (ATSα) domain comprising or consisting of the amino acid sequence set forth as DPDNEAYEMPSEEGYQDYEPEA (SEQ ID NO: 99) fused to the C-terminus of the light chain, the C-terminus of the heavy chain, or both the C-terminus of the light chain and the C-terminus of the heavy chain. ATSα. In some such implementations, the monoclonal antibody comprises a heavy chain and a light chain fused to ATSα domain set forth as any one of: SEQ ID NO: 3 and 79, respectively (VRC07-523LS.ATS1); SEQ ID NO: 80 and 81, respectively (VRC07-523LS.ATS4); SEQ ID NO: 82 and 83, respectively (VRC07-523LS.ATS10); SEQ ID NO: 84 and 85, respectively (N6LS.ATS4); SEQ ID NO: 86 and 87, respectively (N6LS.ATS8); SEQ ID NO: 88 and 89, respectively (N6LS.ATS9); SEQ ID NO: 90 and 91, respectively (10E8.ATS4); SEQ ID NO: 92 and 93, respectively (10E8.ATS11); SEQ ID NO: 94 and 93, respectively (10E8.ATS13); SEQ ID NO: 95 and 79, respectively (VRC01.23LS.ATS1); SEQ ID NO: 96 and 79, respectively (VRC01.23LS.ATS4); or SEQ ID NO: 96 and 83, respectively (VRC01.23LS.ATS5).

The antibody can be of any isotype. The antibody can be, for example, an IgM or an IgG antibody, such as IgG₁, IgG₂, IgG₃, or IgG₄. The class of an antibody that specifically binds HIV-1 Env can be switched with another. In one aspect, a nucleic acid molecule encoding V_{L} or V_{H} is isolated using methods well-known in the art, such that it does not include any nucleic acid sequences encoding the constant region of the light or heavy chain, respectively. A nucleic acid molecule encoding V_{L} or V_{H} is then operatively linked to a nucleic acid sequence encoding a C_{L} or C_{H} from a different class of immunoglobulin molecule. This can be achieved using a vector or nucleic acid molecule that comprises a C_{L} or C_{H} chain, as known in the art. For example, an antibody that specifically binds HIV-1 Env, that was originally IgG may be class switched to an IgM. Class switching can be used to convert one IgG subclass to another, such as from IgG1 to IgG2, IgG3, or IgG4.

In some examples, the disclosed antibodies are oligomers of antibodies, such as dimers, trimers, tetramers, pentamers, hexamers, septamers, octomers and so on.

The antibody or antigen binding fragment can be derivatized or linked to another molecule (such as another peptide or protein). In general, the antibody or antigen binding fragment is derivatized such that the binding to HIV-1 Env is not affected adversely by the derivatization or labeling. For example, the antibody or antigen binding fragment can be functionally linked (by chemical coupling, genetic fusion, noncovalent association or otherwise) to one or more other molecular entities, such as another antibody (for example, a bi-specific antibody or a diabody), a detectable marker, an effector molecule, or a protein or peptide that can mediate association of the antibody or antibody portion with another molecule (such as a streptavidin core region or a polyhistidine tag).

### (a) Binding Affinity

In several implementations, the antibody or antigen binding fragment specifically binds HIV-1 Env with an affinity (e.g., measured by K_{D}) of no more than 1.0×10⁻⁸ M, no more than 5.0×10⁻⁸ M, no more than 1.0×10⁻⁹ M, no more than 5.0×10⁻⁹ M, no more than 1.0×10⁻¹⁰ M, no more than 5.0×10⁻¹⁰ M, or no more than 1.0×10⁻¹¹ M. K_{D} can be measured, for example, by a radiolabeled antigen binding assay (RIA) performed with the Fab version of an antibody of interest and its antigen using known methods. In one assay, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293(4):865-881, 1999). To establish conditions for the assay, MICROTITER® multi-well plates (Thermo Scientific) are coated overnight with 5 g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc™ Catalog #269620), 100 μM or 26 pM [¹²⁵I]-antigen are mixed with serial dilutions of a Fab of interest (e.g., consistent with assessment of the anti-VEGF antibody, Fab-12, in Presta et al., Cancer Res. 57(20):4593-4599, 1997). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 l/well of scintillant (MicroScint™-20; PerkinEmler) is added, and the plates are counted on a TOPCOUNT™ gamma counter (PerkinEmler) for ten minutes. Concentrations of each Fab that give less than or equal to 20% of maximal binding are chosen for use in competitive binding assays.

### (b) Neutralization

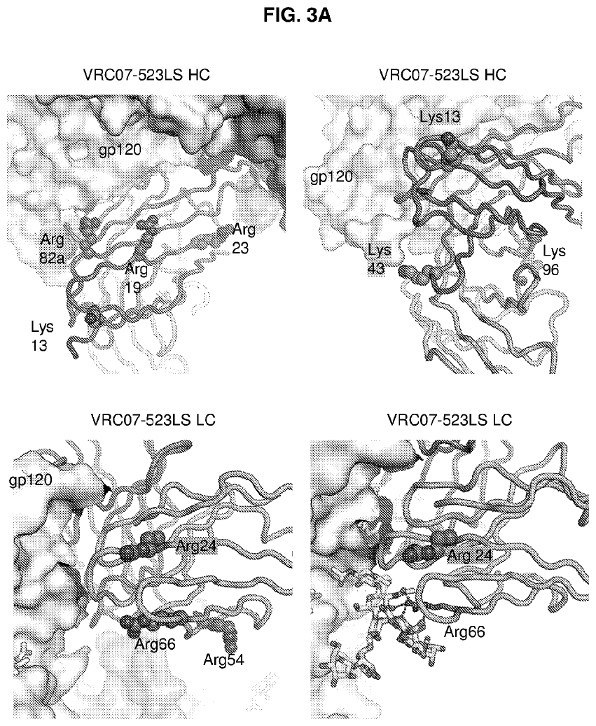

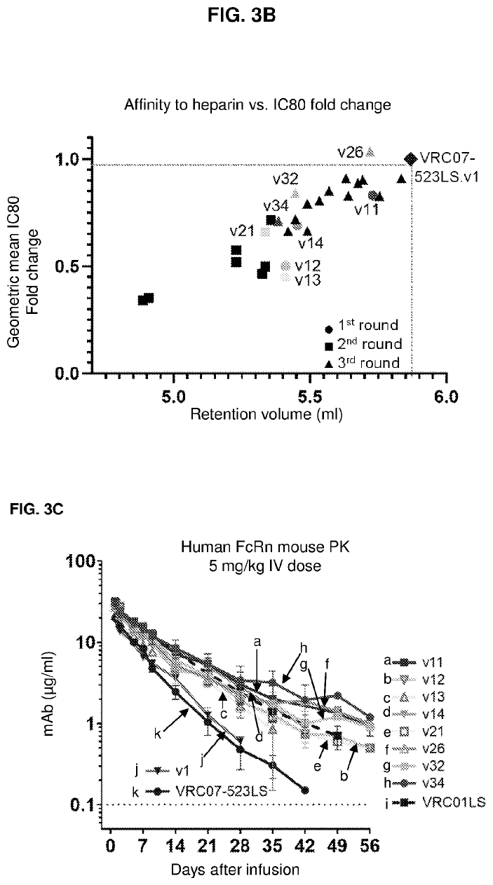

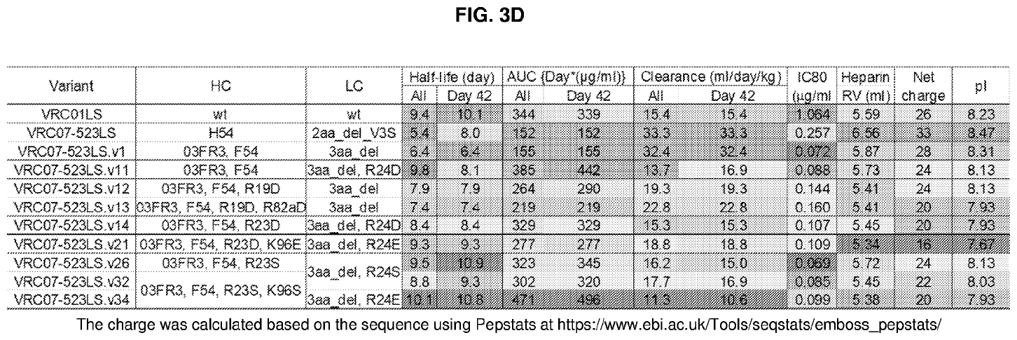

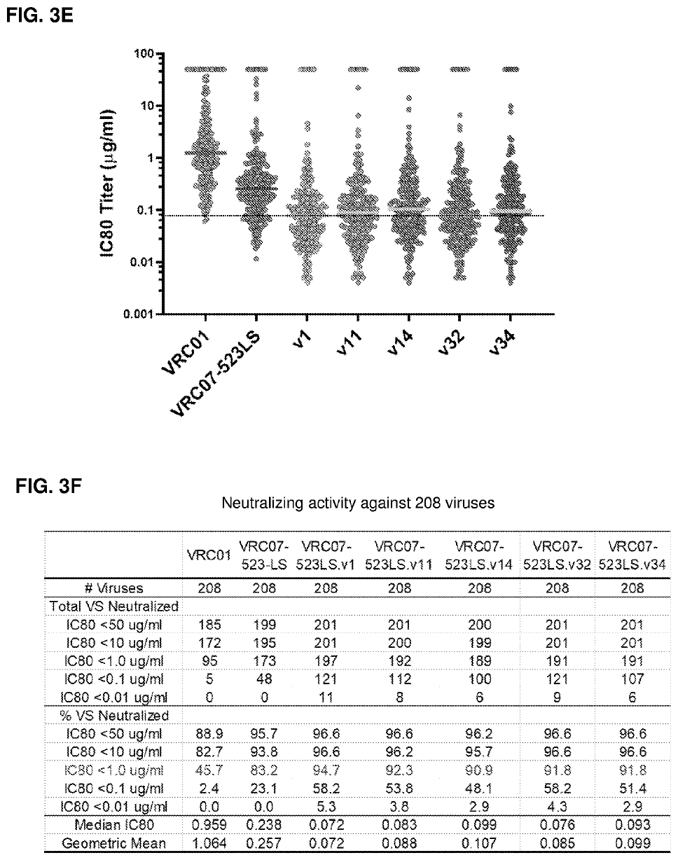

In some implementations, the antibody or antigen binding fragment can also be distinguished by neutralization breadth. In some implementations, the antibody or antigen binding fragment neutralizes at least 80% (such as at least 85%, least 90%, or at least 95%) of the HIV-1 isolates included in a standardized panel of HIV-1 pseudoviruses (such as the panel shown in FIG. 3B) with an IC₅₀ of less than 50 μg/ml. Exemplary pseudovirus neutralization assays and panels of HIV-1 pseudovirus are described for example, in Li et al., J Virol 79, 10108-10125, 2005, incorporated by reference herein. The person of ordinary skill in the art is familiar with methods of measuring neutralization breadth and potency, for example such methods include the single-round HIV-1 Env-pseudoviruses infection of TZM-bl cells (see, e.g., Li et al., J Virol 79, 10108-10125, 2005, incorporated by reference herein; see also, PCT Pub. No. WO2011/038290, incorporated by reference herein).

An additional method to assay for neutralization activity includes a single-cycle infection assay as described in Martin et al. (2003) Nature Biotechnology 21:71-76. In this assay, the level of viral activity is measured via a selectable marker whose activity is reflective of the amount of viable virus in the sample, and the IC₅₀ is determined. In other assays, acute infection can be monitored in the PM1 cell line or in primary cells (normal PBMC). In this assay, the level of viral activity can be monitored by determining the p24 concentrations using ELISA. See, for example, Martin et al. (2003) Nature Biotechnology 21:71-76.

### (c) Multispecific Antibodies

In some implementations, the antibody or antigen binding fragment is included on a multispecific antibody, such as a bi-specific antibody or a tri-specific antibody. Such multispecific antibodies can be produced by known methods, such as crosslinking two or more antibodies, antigen binding fragments (such as scFvs) of the same type or of different types. Exemplary methods of making multispecific antibodies include those described in PCT Pub. No. WO2013/163427, which is incorporated by reference herein in its entirety. Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are commercially available, for example, from Thermo Fisher Scientific, Waltham, MA, and MilliporeSigma Corporation, St. Louis, MO.

In some implementations, a trispecific antibody is provided that includes at least one antibody or antigen binding fragment as disclosed herein. In some implementations, the trispecific antibody three different antigen binding fragments that target HIV-1 Env, such as trispecific antibody format described in Xu et al., “Trispecific broadly neutralizing HIV antibodies mediate potent SHIV protection in macaques,” Science, 358(6359): 85-90, 2017, which is incorporated by reference herein in its entirety.

In some implementations, the antibody or antigen binding fragment is included on a bispecific antibody that that specifically binds to HIV-1 Env and further specifically binds to CD3. Examples of CD3 binding domains that can be included on the bispecific antibody or antigen binding fragment are known and include those disclosed in PCT Pub. No. WO2013/163427, which is incorporated by reference herein in its entirety.

Various types of multi-specific antibodies are known. Bispecific single chain antibodies can be encoded by a single nucleic acid molecule. Examples of bispecific single chain antibodies, as well as methods of constructing such antibodies are known in the art (see, e.g., U.S. Pat. Nos. 8,076,459, 8,017,748, 8,007,796, 7,919,089, 7,820,166, 7,635,472, 7,575,923, 7,435,549, 7,332,168, 7,323,440, 7,235,641, 7,229,760, 7,112,324, 6,723,538, incorporated by reference herein). Additional examples of bispecific single chain antibodies can be found in PCT application No. WO 99/54440; Mack et al., J. Immunol., 158(8):3965-3970, 1997; Mack et al., Proc. Natl. Acad. Sci. U.S.A., 92(15):7021-7025, 1995; Kufer et al., Cancer Immunol. Immunother., 45(3-4):193-197, 1997; Loffler et al., Blood, 95(6):2098-2103, 2000; and Briihl et al., J. Immunol., 166(4):2420-2426, 2001. Production of bispecific Fab-scFv (“bibody”) molecules are described, for example, in Schoonjans et al. (J. Immunol., 165(12):7050-7057, 2000) and Willems et al. (J. Chromatogr. B Analyt. Technol. Biomed Life Sci. 786(1-2):161-176, 2003). For bibodies, a scFv molecule can be fused to one of the VL-CL (L) or VH-CH1 chains, e.g., to produce a bibody one scFv is fused to the C-term of a Fab chain.

### (d) Fragments

Antigen binding fragments are encompassed by the present disclosure, such as Fab, F(ab′)₂, and Fv which include a heavy chain and V_{L} and specifically bind HIV-1 Env. These antibody fragments retain the ability to selectively bind with the antigen and are “antigen-binding” fragments. Non-limiting examples of such fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the VH and V_{L} expressed as two chains; and
(5) Single chain antibody (such as scFv), defined as a genetically engineered molecule containing the V_{H} and the V_{L} linked by a suitable polypeptide linker as a genetically fused single chain molecule (see, e.g., Ahmad et al., Clin. Dev. Immunol., 2012, doi:10.1155/2012/980250; Marbry and Snavely, IDrugs, 13(8):543-549, 2010). The intramolecular orientation of the V_{H}-domain and the V_{L}-domain in a scFv, is not decisive for the provided antibodies (e.g., for the provided multispecific antibodies). Thus, scFvs with both possible arrangements (V_{H}-domain-linker domain-V_{L}-domain; V_{L}-domain-linker domain-V_{H}-domain) may be used.
(6) A dimer of a single chain antibody (scFV₂), defined as a dimer of a scFV. This has also been termed a “miniantibody.”

Methods of making these fragments are known in the art (see for example, Greenfield (Ed.), Antibodies: A Laboratory Manual, 2^{nd} ed. New York: Cold Spring Harbor Laboratory Press, 2014).

Antigen binding fragments can be prepared by proteolytic hydrolysis of the antibody or by expression in a host cell (such as an E. coli cell) of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. In some examples, the antibody heavy chain can include an engineered protease cleave site (such as an HRV3C protease cleavage site) in place of or in addition to the typical papain cleavage site to facilitate cleavage by proteases other than papain.

### (e) Additional Description of Antibody Variants

In some implementations, amino acid sequence variants of the antibodies provided herein are provided. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some implementations, antibody variants having one or more amino acid substitutions are provided. In a non-limiting example, sites of interest for substitutional mutagenesis include the framework regions. Amino acid substitutions may be introduced into an antibody of interest and the products screened for a desired activity, e.g., retained/improved antigen binding, increased HIV-1 neutralization breadth or potency, decreased immunogenicity, or improved antibody dependent cellular cytotoxicity (ADCC) or complement dependent cytotoxicity (CDC).

The variants typically retain amino acid residues necessary for correct folding and stabilizing between the V_{H} and the V_{L} regions, and will retain the charge characteristics of the residues in order to preserve the low pI and low toxicity of the molecules. Amino acid substitutions can be made in the V_{H} and the V_{L} regions to increase yield.

In some implementations, an antibody or antigen binding fragment is altered to increase or decrease the extent to which the antibody or antigen binding fragment is glycosylated. Addition or deletion of glycosylation sites may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH₂ domain of the Fc region. See, e.g., Wright et al. Trends Biotechnol. 15(1):26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some implementations, modifications of the oligosaccharide in an antibody may be made in order to create antibody variants with certain improved properties.

In one implementation, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region; however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO 2002/031140; Okazaki et al., J. Mol. Biol., 336(5):1239-1249, 2004; Yamane-Ohnuki et al., Biotechnol. Bioeng. 87(5):614-622, 2004. Examples of cell lines capable of producing defucosylated antibodies include Lee 13 CHO cells deficient in protein fucosylation (Ripka et al., Arch. Biochem. Biophys. 249(2):533-545, 1986; US Pat. Appl. No. US 2003/0157108 and WO 2004/056312, especially at Example 11), and knockout cell lines, such as alpha-1,6-fucosyltransferase gene, FUT8, knockout CHO cells (see, e.g., Yamane-Ohnuki et al., Biotechnol. Bioeng., 87(5): 614-622, 2004; Kanda et al., Biotechnol. Bioeng., 94(4):680-688, 2006; and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In several implementations, the constant region of the antibody comprises one or more amino acid substitutions to optimize in vivo half-life of the antibody. The serum half-life of IgG Abs is regulated by the neonatal Fc receptor (FcRn). Thus, in several implementations, the antibody comprises an amino acid substitution that increases binding to the FcRn. Several such substitutions are known, such as substitutions at IgG constant regions T250Q and M428L (see, e.g., Hinton et al., J Immunol., 176(1):346-356, 2006); M428L and N434S (the “LS” mutation, see, e.g., Zalevsky, et al., Nature Biotechnol., 28(2):157-159, 2010); N434A (see, e.g., Petkova et al., Int. Immunol., 18(12):1759-1769, 2006); T307A, E380A, and N434A (see, e.g., Petkova et al., Int. Immunol., 18(12):1759-1769, 2006); and M252Y, S254T, and T256E (see, e.g., Dall'Acqua et al., J. Biol. Chem., 281(33):23514-23524, 2006). The disclosed antibodies and antigen binding fragments can be linked to or comprise a Fc polypeptide including any of the substitutions listed above, for example, the Fc polypeptide can include the M428L and N434S substitutions (EU numbering). The M428L and N434S substitutions (EU numbering) are equivalent to M459L and N465S substitutions (Kabat numbering). Exemplary sequences of an IgG1 constant region containing the M428L and N434S substitutions are provided herein, which can be paired with an appropriate light chain constant region, and appropriate V_{H} and V_{L} regions as provided herein to generate a monoclonal antibody. In a non-limiting example, the monoclonal antibody comprises heavy and light chains comprising the amino acid sequences set forth as SEQ ID NOs: 91 and 92, respectively, or 99 and 100, respectively. As used herein, reference to an antibody with the “LS” substitution (or similar language) indicates that the antibody heavy chain is an IgG with M428L and N434S substitutions.

In some implementations, the constant region of the antibody comprises one or more amino acid substitutions to optimize ADCC. ADCC is mediated primarily through a set of closely related Fcγ receptors. In some implementations, the antibody comprises one or more amino acid substitutions that increase binding to FcγRIIIa. Several such substitutions are known, such as substitutions at IgG constant regions S239D and 1332E (see, e.g., Lazar et al., Proc. Natl., Acad. Sci. U.S.A., 103(11):4005-4010, 2006); and S239D, A330L, and 1332E (see, e.g., Lazar et al., Proc. Natl., Acad. Sci. U.S.A., 103(11):4005-4010, 2006).

In some implementations, the constant region of the antibody is modified to improve pharmacokinetics. In some implementations, for IgG1 antibodies, the heavy chain of the antibody comprises K129E, K222E, and K228E substitutions according to the Kabat numbering system. In some implementations, for IgG1 antibodies, the light chain is a kappa light chain and comprises K126E, K145E, K188E, K190E substitutions according to the Kabat numbering system. In some implementations, for IgG1 antibodies, the light chain is a lambda light chain and comprises K166E, K187E, R190E, and K207E substitutions according to the Kabat numbering system. In some implementations, for IgG1 antibodies, the heavy chain of the antibody comprises K129E, K222E, and K228E substitutions according to the Kabat numbering system, and the light chain is a kappa light chain and comprises K126E, K145E, K188E, K190E substitutions according to the Kabat numbering system, or is a lambda light and comprises K166E, K187E, R190E, and K207E substitutions according to the Kabat numbering system.

Combinations of the above substitutions are also included, to generate an IgG constant region with increased binding to FcRn and FcγRIIIa. The combinations increase antibody half-life and ADCC. For example, such combinations include antibodies with the following amino acid substitutions in the Fc region: (1) S239D/I332E and T250Q/M428L; (2) S239D/I332E and M428L/N434S; (3) S239D/I332E and N434A; (4) S239D/I332E and T307A/E380A/N434A; (5) S239D/I332E and M252Y/S254T/T256E; (6) S239D/A330L/I332E and 250Q/M428L; (7) S239D/A330L/I332E and M428L/N434S; (8) S239D/A330L/I332E and N434A; (9) S239D/A330L/I332E and T307A/E380A/N434A; or (10) S239D/A330L/I332E and M252Y/S254T/T256E.

In some examples, the antibodies, or an antigen binding fragment thereof is modified such that it is directly cytotoxic to infected cells, or uses natural defenses such as complement, ADCC, or phagocytosis by macrophages.

In some implementations, an antibody provided herein may be further modified to contain additional nonproteinaceous moieties that are known in the art and readily available. The moieties suitable for derivatization of the antibody include but are not limited to water soluble polymers.

Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in an application under defined conditions, etc.

(f) Antibody Constant Regions with Modifications to Improve PK

In some implementations, a monoclonal antibody is provided that comprises a constant region that is modified to improve pharmacokinetics. In some implementations, for IgG1 antibodies, the heavy chain of the antibody comprises K129E, K222E, and K228E substitutions according to the Kabat numbering system. In some implementations, for IgG1 antibodies, the light chain is a kappa light chain and comprises K126E, K145E, K188E, K190E substitutions according to the Kabat numbering system. In some implementations, for IgG1 antibodies, the light chain is a lambda light chain and comprises K166E, K187E, R190E, and K207E substitutions according to the Kabat numbering system. In some implementations, for IgG1 antibodies, the heavy chain of the antibody comprises K129E, K222E, and K228E substitutions according to the Kabat numbering system, and the light chain is a kappa light chain and comprises K126E, K145E, K188E, K190E substitutions according to the Kabat numbering system, or is a lambda light and comprises K166E, K187E, R190E, and K207E substitutions according to the Kabat numbering system.

In some implementations, for IgG1 antibodies comprising a heavy chain comprising the K129E, K222E, and K228E substitutions (according to the Kabat), the heavy chain comprises a constant region comprising the amino acid sequence set forth as SEQ ID NO: 114:

ASTKGPSVFPLAPSSESTSGGTAALGCLVKDYFPEPVTVSWNSGALTSGV
HTFPAVLQSSGLYSLSSVVTVPSSSLGTQTYICNVNHKPSNTKVDKEVEP
ESCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVLHEALHSHYTQKSLSLSPGK

In some implementations, for IgG1 antibodies comprising a kappa light chain comprising K126E, K145E, K188E, K190E substitutions (according to Kabat), the light chain comprises a constant region comprising the amino acid sequence set forth as SEQ ID NO: 115:

LTQSPGTLSLSPGETAIISCRTSQYGSLAWYQQRPGQAPRLVIYSGSTRA
AGIPDRFSGSRWGPDYNLTISNLESGDFGVYYCQQYEFFGQGTKVQVDIK
RTVAAPSVFIFPPSDEQLESGTASVVCLLNNFYPREAEVQWKVDNALQSG
NSQESVTEQDSKDSTYSLSSTLTLSKADYEEHEVYACEVTHQGLSSPVTK
SFNRGEC

In some implementations, for IgG1 antibodies comprising a lambda light chain comprising K166E, K187E, R190E, and K207E substitutions (according to Kabat), the light chain comprises a constant region comprising the amino acid sequence set forth as SEQ ID NO: 116:

ASELTQDPAVSVALKQTVTITCRGDSLRSHYASWYQKKPGQAPVLLFYGK
NNRPSGIPDRFSGSASGNRASLTITGAQAEDEADYYCSSRDKSGSRLSVF
GGGTKLTVLSQPKAAPSVTLFPPSSEELQANKATLVCLISDFYPGAVTVA
WKADSSPVKAGVETTTPSEQSNNKYAASSYLSLTPEQWESHESYSCQVTH
EGSTVEETVAPTECS

### B. Conjugates

The antibodies and antigen binding fragments described herein (e.g., that specifically bind to an epitope on gp120) can be conjugated to an agent, such as an effector molecule or detectable marker. Both covalent and noncovalent attachment means may be used. Various effector molecules and detectable markers can be conjugated to the antibody or antigen binding fragment, including (but not limited to) toxins and radioactive agents such as ¹²⁵I, ³²P, ¹⁴C, ³H and ³⁵S and other labels, target moieties and ligands, etc. The choice of a particular effector molecule or detectable marker depends on the particular target molecule or cell, and the desired biological effect.

The choice of a particular effector molecule or detectable marker depends on the particular target molecule or cell, and the desired biological effect. Thus, for example, the effector molecule can be a cytotoxin that is used to bring about the death of a particular target cell (such as an HIV-1 infected cell). In other implementations, the effector molecule can be a cytokine, such as IL-15; conjugates including the cytokine can be used, e.g., to stimulate immune cells locally.

The procedure for attaching an effector molecule or detectable marker to an antibody or antigen binding fragment varies according to the chemical structure of the effector. Polypeptides typically contain a variety of functional groups, such as carboxyl (—COOH), free amine (—NH₂) or sulfhydryl (—SH) groups, which are available for reaction with a suitable functional group on a polypeptide to result in the binding of the effector molecule or detectable marker. Alternatively, the antibody or antigen binding fragment is derivatized to expose or attach additional reactive functional groups. The derivatization may involve attachment of any of a number of known linker molecules, such as those available from Thermo Fisher Scientific, Waltham, MA and MilliporeSigma Corporation, St. Louis, MO. The linker is capable of forming covalent bonds to both the antibody or antigen binding fragment and to the effector molecule or detectable marker. Suitable linkers include, but are not limited to, straight or branched-chain carbon linkers, heterocyclic carbon linkers, or peptide linkers. Where the antibody or antigen binding fragment and the effector molecule or detectable marker are polypeptides, the linkers may be joined to the constituent amino acids through their side chains (such as through a disulfide linkage to cysteine) or the alpha carbon, or through the amino, and/or carboxyl groups of the terminal amino acids.

In view of the large number of methods that have been reported for attaching a variety of radiodiagnostic compounds, radiotherapeutic compounds, labels (such as enzymes or fluorescent molecules), toxins, and other agents to antibodies, a suitable method for attaching a given agent to an antibody or antigen binding fragment or other polypeptide can be determined.

In some implementations, the antibody or antigen binding fragment can be conjugated with effector molecules such as small molecular weight drugs such as Monomethyl Auristatin E (MMAE), Monomethyl Auristatin F (MMAF), maytansine, maytansine derivatives, including the derivative of maytansine known as DM1 (also known as mertansine), or other agents to make an antibody drug conjugate (ADC). In several implementations, conjugates of an antibody or antigen binding fragment and one or more small molecule toxins, such as a calicheamicin, maytansinoids, dolastatins, auristatins, a trichothecene, and CC1065, and the derivatives of these toxins that have toxin activity, are provided.

The antibody or antigen binding fragment can be conjugated with a detectable marker; for example, a detectable marker capable of detection by ELISA, spectrophotometry, flow cytometry, microscopy or diagnostic imaging techniques (such as CT, computed axial tomography (CAT), MRI, magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP), and yellow fluorescent protein (YFP). An antibody or antigen binding fragment can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present, the addition of hydrogen peroxide and diaminobenzidine leads to a colored reaction product, which is visually detectable. An antibody or antigen binding fragment may also be conjugated with biotin, and detected through indirect measurement of avidin or streptavidin binding. It should be noted that the avidin itself can be conjugated with an enzyme or a fluorescent label.

The antibody or antigen binding fragment can be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody or antigen binding fragment may also be labeled with a predetermined polypeptide epitope recognized by a secondary reporter (such as leucine zipper pair sequences, binding sites for secondary antibodies, metal binding domains, epitope tags).

The antibody or antigen binding fragment can be conjugated with a radiolabeled amino acid, for example, for diagnostic purposes. For instance, the radiolabel may be used to detect gp120 and gp120 expressing cells by radiography, emission spectra, or other diagnostic techniques. Examples of labels for polypeptides include, but are not limited to, the following radioisotopes: ³H, ¹⁴C ³⁵S, ⁹⁰Y, ^{99m}Tc, ¹¹¹In, ¹²⁵I, ¹³¹I. The radiolabels may be detected, for example, using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of effector molecule or detectable marker moieties per antibody or antigen binding fragment in a conjugate can range, for example, from 1 to 20 moieties per antibody or antigen binding fragment. In some implementations, the average number of effector molecules or detectable marker moieties per antibody or antigen binding fragment in a conjugate range from about 1 to about 2, from about 1 to about 3, about 1 to about 8; from about 2 to about 6; from about 3 to about 5; or from about 3 to about 4. The loading (for example, effector molecule per antibody ratio) of a conjugate may be controlled in different ways, for example, by: (i) limiting the molar excess of effector molecule-linker intermediate or linker reagent relative to antibody, (ii) limiting the conjugation reaction time or temperature, (iii) partial or limiting reducing conditions for cysteine thiol modification, (iv) engineering by recombinant techniques the amino acid sequence of the antibody such that the number and position of cysteine residues is modified for control of the number or position of linker-effector molecule attachments.

### C. Polynucleotides and Expression

Nucleic acid molecules (for example, cDNA or RNA molecules) encoding the amino acid sequences of antibodies, antigen binding fragments, and conjugates described herein (e.g., that specifically bind HIV-1 Env) are provided. Nucleic acids encoding these molecules can readily be produced using the amino acid sequences provided herein (such as the CDR sequences and V_{H} and V_{L} sequences), sequences available in the art (such as framework or constant region sequences), and the genetic code. In several implementations, nucleic acid molecules can encode the V_{H}, the V_{L}, or both the V_{H} and V_{L} (for example in a bicistronic expression vector) of a disclosed antibody or antigen binding fragment. In several implementations, the nucleic acid molecules can be expressed in a host cell (such as a mammalian cell) to produce a disclosed antibody or antigen binding fragment.

The genetic code can be used to construct a variety of functionally equivalent nucleic acid sequences, such as nucleic acids which differ in sequence but which encode the same antibody sequence or a conjugate or fusion protein including the V_{L} and/or V_{H} of the antibody.

Nucleic acid molecules encoding the antibodies, antigen binding fragments, and conjugates that specifically bind HIV-1 Env can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as bacteria, plant, yeast, insect and mammalian cells. The antibodies, antigen binding fragments, and conjugates can be expressed as individual proteins including the V_{H} and/or V_{L} (linked to an effector molecule or detectable marker as needed), or can be expressed as a fusion protein. Methods of expressing and purifying antibodies and antigen binding fragments are known and further described herein (see, e.g., Al-Rubeai (Ed.), Antibody Expression and Production, Dordrecht; New York: Springer, 2011). An immunoadhesin can also be expressed. Thus, in some examples, nucleic acids encoding a V_{H} and V_{L}, and immunoadhesin are provided. The nucleic acid sequences can optionally encode a leader sequence.

To create a scFv the V_{H}- and V_{L}-encoding DNA fragments can be operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} domains joined by the flexible linker (see, e.g., Bird et al., Science, 242(4877):423-426, 1988; Huston et al., Proc. Natl. Acad. Sci. U.S.A., 85(16):5879-5883, 1988; McCafferty et al., Nature, 348:552-554, 1990; Kontermann and Dübel (Eds.), Antibody Engineering, Vols. 1-2, _{2nd}ed., Springer-Verlag, 2010; Greenfield (Ed.), Antibodies: A Laboratory Manual, 2^{nd} ed. New York: Cold Spring Harbor Laboratory Press, 2014). Optionally, a cleavage site can be included in a linker, such as a furin cleavage site.

The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to gp120 and another antigen, such as, but not limited to CD3. The encoded V_{H} and V_{L} optionally can include a furin cleavage site between the V_{H} and V_{L} domains.

One or more DNA sequences encoding the antibodies, antigen binding fragments, or conjugates can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells such as the COS, CHO, HeLa and myeloma cell lines, can be used to express the disclosed antibodies and antigen binding fragments. Methods of stable transfer, meaning that the foreign DNA is continuously maintained in the host, are known in the art. Hybridomas expressing the antibodies of interest are also encompassed by this disclosure.

The expression of nucleic acids encoding the antibodies and antigen binding fragments described herein can be achieved by operably linking the DNA or cDNA to a promoter (which is either constitutive or inducible), followed by incorporation into an expression cassette. The promoter can be any promoter of interest, including a cytomegalovirus promoter and a human T cell lymphotrophic virus promoter (HTLV)-1. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lambda promoters, a ribosome binding site, and preferably a transcription termination signal.

For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by well-known methods such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, GPt, neo, and hyg genes.

Modifications can be made to a nucleic acid encoding a polypeptide described herein without diminishing its biological activity. Some modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the antibodies, antigen binding fragments, and conjugates can be purified according to standard procedures in the art, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The antibodies, antigen binding fragment, and conjugates need not be 100% pure. Once purified, partially or to homogeneity as desired, if to be used prophylatically, the polypeptides should be substantially free of endotoxin.

Methods for expression of antibodies, antigen binding fragments, and conjugates, and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341(6242):544-546, 1989.

### D. Methods and Compositions

### 1. Therapeutic Methods

Methods are disclosed herein for the inhibition of an HIV-1 infection in a subject. The methods include administering to a subject an effective amount (that is, an amount effective to inhibit HIV-1 infection in a subject) of a disclosed antibody, antigen binding fragment, conjugate, or a nucleic acid encoding such an antibody, antigen binding fragment, or conjugate, to a subject with or at risk of the HIV-1 infection. The methods can be used pre-exposure (for example, to prevent HIV-1 infection), in post-exposure prophylaxis, or for treatment of a subject with an HIV-1 infection.

In some examples, the antibody, antigen binding fragment, conjugate, or nucleic acid molecule, can be used to eliminate or reduce the viral reservoir of HIV-1 in a subject.

HIV-1 infection does not need to be completely inhibited for the method to be effective. For example, the method can decrease HIV-1 infection by at least 10%, at least 20%, at least 50%, at least 60%, at least 70%, at least 80%, at least 90%, at least 95%, at least 98%, or even at least 100% (elimination of detectable HIV-1 infected cells), as compared to HIV-1 infection in the absence of the treatment. In some implementations, the method results in a reduction of HIV-1 replication in the subject. HIV-1 replication does not need to be completely eliminated for the method to be effective. For example, the method can reduce HIV-1 replication in the subject by at least 10%, at least 20%, at least 50%, at least 60%, at least 70%, at least 80%, at least 90%, at least 95%, at least 98%, or even at least 100% (elimination of detectable HIV-1 replication), as compared to HIV-1 replication in the absence of the treatment.

In some implementations, administration of an effective amount of a disclosed antibody, antigen binding fragment, conjugate, or nucleic acid molecule, inhibits the establishment of HIV-1 infection and/or subsequent HIV-1 progression in a subject, which can encompass any statistically significant reduction in HIV-1 activity or symptoms of HIV-1 infection in the subject.

In one implementation, administration of a disclosed antibody, antigen binding fragment, conjugate, or nucleic acid molecule, results in a reduction in the establishment of HIV-1 infection and/or reducing subsequent HIV-1 disease progression in a subject. A reduction in the establishment of HIV-1 infection and/or a reduction in subsequent HIV-1 disease progression encompass any statistically significant reduction in HIV-1 activity. In some implementations, methods are disclosed for treating a subject with an HIV-1 infection. These methods include administering to the subject a effective amount of a disclosed antibody, antigen binding fragment, conjugate, or nucleic acid molecule, to preventing or treating the HIV-1 infection.

Studies have shown that the rate of HIV-1 transmission from mother to infant is reduced significantly when zidovudine is administered to HIV-infected women during pregnancy and delivery and to the offspring after birth (Connor et al., 1994 Pediatr Infect Dis J 14: 536-541). The present disclosure provides antibodies, antigen binding fragments, conjugates, and nucleic acid molecule that are of use in decreasing HIV-transmission from mother to infant. In some examples, an effective amount of a HIV-1 Env-specific antibody or antigen binding fragment thereof or nucleic acid encoding such antibodies or antibody antigen binding fragments, is administered to a pregnant subject in order to prevent transmission of HIV-1, or decrease the risk of transmission of HIV-1, from a mother to an infant. In some examples, an effective amount of the antibody, or an antigen binding fragment or nucleic acid encoding such antibodies or antigen binding fragment, is administered to mother and/or to the child at childbirth. In other examples, an effective amount of the antibody, antigen binding fragment, or nucleic acid encoding the antibody or antigen binding fragment is administered to the mother and/or infant prior to breast feeding in order to prevent viral transmission to the infant or decrease the risk of viral transmission to the infant.

For any application, the antibody, antigen binding fragment, conjugate, or nucleic acid molecule can be combined with anti-retroviral therapy. Antiretroviral drugs are broadly classified by the phase of the retrovirus life-cycle that the drug inhibits. The disclosed antibodies can be administered in conjunction with nucleoside analog reverse-transcriptase inhibitors (such as zidovudine, didanosine, zalcitabine, stavudine, lamivudine, abacavir, emtricitabine, entecavir, and apricitabine), nucleotide reverse transcriptase inhibitors (such as tenofovir and adefovir), non-nucleoside reverse transcriptase inhibitors (such as efavirenz, nevirapine, delavirdine, etravirine, and rilpivirine), protease inhibitors (such as saquinavir, ritonavir, indinavir, nelfinavir, amprenavir, lopinavir, fosamprenavir, atazanavir, tipranavir, and darunavir), entry or fusion inhibitors (such as maraviroc and enfuvirtide), maturation inhibitors, (such as bevirimat and vivecon), or a broad spectrum inhibitors, such as natural antivirals. In some examples, a disclosed antibody or active fragment thereof or nucleic acids encoding such is administered in conjunction with IL-15, or conjugated to IL-15.

Studies have shown that cocktails of HIV-1 neutralizing antibodies that target different epitopes of gp120 can treat macaques chronically infected with SHIV (Shingai et al., Nature, 503, 277-280, 2013; and Barouch et al., Nature, 503, 224-228, 2013). Accordingly, in some examples, a subject is further administered one or more additional antibodies that bind HIV-1 Env (e.g., that bind to gp120 or gp41), and that can neutralize HIV-1. The additional antibodies can be administrated before, during, or after administration of the novel antibodies disclosed herein. In some implementations, the additional antibody can be an antibody that specifically binds to an epitope on HIV-1 Env such as the membrane-proximal external region (e.g., 10E8 antibody), the V1/V2 domain (e.g., PG9 antibody, CAP256-VRC26), or the V3 loop (e.g., 10⁻¹⁰⁷⁴, PGT 121, or PGT128 antibody), or those that bind both gp120 and gp41 subunits (eg. 35022, PGT151, or 8ANC195). Antibodies that specifically bind to these regions and neutralizing HIV-1 infection are known to the person of ordinary skill in the art. Non-limiting examples can be found, for example, in PCT Pub. No. WO 2011/038290, WO/2013/086533, WO/2013/090644, WO/2012/158948, which are incorporated herein by reference in their entirety.

Antibodies and antigen binding fragments thereof are typically administered by intravenous infusion. Doses of the antibody or antigen binding fragment vary, but generally range between about 0.5 mg/kg to about 50 mg/kg, such as a dose of about 1 mg/kg, about 5 mg/kg, about 10 mg/kg, about 20 mg/kg, about 30 mg/kg, about 40 mg/kg, or about 50 mg/kg. In some implementations, the dose of the antibody or antigen binding fragment can be from about 0.5 mg/kg to about 5 mg/kg, such as a dose of about 1 mg/kg, about 2 mg/kg, about 3 mg/kg, about 4 mg/kg or about 5 mg/kg. The antibody or antigen binding fragment is administered according to a dosing schedule determined by a medical practitioner. In some examples, the antibody or antigen binding fragment is administered weekly, every two weeks, every three weeks or every four weeks.

In some examples, a subject is administered DNA or RNA encoding a disclosed antibody to provide in vivo antibody production, for example using the cellular machinery of the subject. Administration of nucleic acid constructs is known in the art and taught, for example, in U.S. Pat. Nos. 5,643,578, 5,593,972 and 5,817,637. U.S. Pat. No. 5,880,103 describes several methods of delivery of nucleic acids encoding proteins to an organism. One approach to administration of nucleic acids is direct administration with plasmid DNA, such as with a mammalian expression plasmid. The nucleotide sequence encoding the disclosed antibody, or antigen binding fragments thereof, can be placed under the control of a promoter to increase expression. The methods include liposomal delivery of the nucleic acids. Such methods can be applied to the production of an antibody, or antigen binding fragments thereof. In some implementations, a disclosed antibody or antigen binding fragment is expressed in a subject using the pVRC8400 vector (described in Barouch et al., J. Virol., 79(14), 8828-8834, 2005, which is incorporated by reference herein).

In several implementations, a subject (such as a human subject at risk of ebolavirus infection) can be administered an effective amount of an AAV viral vector that includes one or more nucleic acid molecules encoding a disclosed antibody or antigen binding fragment. The AAV viral vector is designed for expression of the nucleic acid molecules encoding a disclosed antibody or antigen binding fragment, and administration of the effective amount of the AAV viral vector to the subject leads to expression of an effective amount of the antibody or antigen binding fragment in the subject. Non-limiting examples of AAV viral vectors that can be used to express a disclosed antibody or antigen binding fragment in a subject include those provided in Johnson et al., Nat. Med., 15(8):901-906, 2009 and Gardner et al., Nature, 519(7541):87-91, 2015, each of which is incorporated by reference herein in its entirety.

In one implementation, a nucleic acid encoding a disclosed antibody, or antigen binding fragment thereof, is introduced directly into tissue. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 g/kg to about 50 mg/kg, and typically are about 0.005 mg/kg to about 5 mg/kg (see, e.g., U.S. Pat. No. 5,589,466).

Single or multiple administrations of a composition including a disclosed HIV-1 Env specific antibody, antigen binding fragment, conjugate, or nucleic acid molecule encoding such molecules, can be administered depending on the dosage and frequency as required and tolerated by the patient. The dosage can be administered once, but may be applied periodically until either a desired result is achieved or until side effects warrant discontinuation of therapy. Generally, the dose is sufficient to inhibit ebolavirus infection without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The effective dose can be determined from cell culture assays and animal studies.

The HIV-1 Env-specific antibody, antigen binding fragment, conjugate, or nucleic acid molecule encoding such molecules, or a composition including such molecules, can be administered to subjects in various ways, including local and systemic administration, such as, e.g., by injection subcutaneously, intravenously, intra-arterially, intraperitoneally, intramuscularly, intradermally, or intrathecally. In an implementation, the antibody, antigen binding fragment, conjugate, or nucleic acid molecule encoding such molecules, or a composition including such molecules, is administered by a single subcutaneous, intravenous, intra-arterial, intraperitoneal, intramuscular, intradermal or intrathecal injection once a day. The antibody, antigen binding fragment, conjugate, or nucleic acid molecule encoding such molecules, or a composition including such molecules, can also be administered by direct injection at or near the site of disease. A further method of administration is by osmotic pump (e.g., an Alzet pump) or mini-pump (e.g., an Alzet mini-osmotic pump), which allows for controlled, continuous and/or slow-release delivery of the antibody, antigen binding fragment, conjugate, or nucleic acid molecule encoding such molecules, or a composition including such molecules, over a pre-determined period. The osmotic pump or mini-pump can be implanted subcutaneously, or near a target site. 2. Compositions Compositions are provided that include one or more of the antibodies, antigen binding fragment, or conjugate (e.g., that specifically bind to HIV-1 gp120) provided herein, or nucleic acid molecule encoding such molecules, in a carrier. The compositions are useful, for example, for the inhibition or detection of an HIV-1 infection. The compositions can be prepared in unit dosage forms for administration to a subject. The amount and timing of administration are at the discretion of the administering physician to achieve the desired purposes. The HIV-1 Env-specific antibody, antigen binding fragment, conjugate, or nucleic acid molecule encoding such molecules can be formulated for systemic or local administration. In one example, the HIV-1 Env-specific antibody, antigen binding fragment, conjugate, or nucleic acid molecule encoding such molecules, is formulated for parenteral administration, such as intravenous administration.

In some implementations, the antibody, antigen binding fragment, or conjugate thereof, in the composition is at least 70% (such as at least 75%, at least 80%, at least 85%, at least 90%, at least 95%, at least 96%, at least 97%, at least 98% or at least 99%) pure. In some implementations, the composition contains less than 10% (such as less than 5%, less than 4%, less than 3%, less than 2%, less than 1%, less than 0.5%, or even less) of macromolecular contaminants, such as other mammalian (e.g., human) proteins.

The compositions for administration can include a solution of the HIV-1 Env-specific antibody, antigen binding fragment, conjugate, or nucleic acid molecule encoding such molecules, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical composition for intravenous administration includes about 0.01 to about 30 mg/kg of antibody or antigen binding fragment or conjugate per subject per day (or the corresponding dose of a conjugate including the antibody or antigen binding fragment). Actual methods for preparing administrable compositions are known and are described in more detail in such publications as Remington: The Science and Practice of Pharmacy, 22^{nd} ed., London, UK: Pharmaceutical Press, 2013. In some implementations, the composition can be a liquid formulation including one or more antibodies, antigen binding fragments (such as an antibody or antigen binding fragment that specifically binds to HIV-1 Env), in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

Antibodies, or an antigen binding fragment thereof or a conjugate or a nucleic acid encoding such molecules, can be provided in lyophilized form and rehydrated with sterile water before administration, although they are also provided in sterile solutions of known concentration. The antibody solution, or an antigen binding fragment or a nucleic acid encoding such antibodies or antigen binding fragments, can then be added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of Rituximab in 1997. Antibodies, antigen binding fragments, conjugates, or a nucleic acid encoding such molecules, can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled-release parenteral formulations can be made as implants, oily injections, or as particulate systems. For a broad overview of protein delivery systems see, Banga, Therapeutic Peptides and Proteins: Formulation, Processing, and Delivery Systems, Lancaster, PA: Technomic Publishing Company, Inc., 1995. Particulate systems include microspheres, microparticles, microcapsules, nanocapsules, nanospheres, and nanoparticles. Microcapsules contain the active protein agent, such as a cytotoxin or a drug, as a central core. In microspheres, the active protein agent is dispersed throughout the particle. Particles, microspheres, and microcapsules smaller than about 1 m are generally referred to as nanoparticles, nanospheres, and nanocapsules, respectively. Capillaries have a diameter of approximately 5 m so that only nanoparticles are administered intravenously. Microparticles are typically around 100 m in diameter and are administered subcutaneously or intramuscularly. See, for example, Kreuter, Colloidal Drug Delivery Systems, J. Kreuter (Ed.), New York, NY: Marcel Dekker, Inc., pp. 219-342, 1994; and Tice and Tabibi, Treatise on Controlled Drug Delivery: Fundamentals, Optimization, Applications, A. Kydonieus (Ed.), New York, NY: Marcel Dekker, Inc., pp. 315-339, 1992.

Polymers can be used for ion-controlled release of the antibody compositions disclosed herein. Various degradable and nondegradable polymeric matrices for use in controlled drug delivery are known in the art (Langer, Acc. Chem. Res. 26(10):537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res., 9(3):425-434, 1992; and Pec et al., J. Parent. Sci. Tech., 44(2):58-65, 1990). Alternatively, hydroxyapatite has been used as a microcarrier for controlled release of proteins (Ijntema et al., Int. J. Pharm. 112(3):215-224, 1994). In yet another aspect, liposomes are used for controlled release as well as drug targeting of the lipid-capsulated drug (Betageri et al., Liposome Drug Delivery Systems, Lancaster, PA: Technomic Publishing Co., Inc., 1993). Numerous additional systems for controlled delivery of active protein agent are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

### 3. Methods of Detection and Diagnosis

Methods are also provided for the detection of the presence of HIV-1 Env in vitro or in vivo. In one example, the presence of HIV-1 Env is detected in a biological sample from a subject, and can be used to identify a subject with HIV-1 infection. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. The method of detection can include contacting a cell or sample, with an antibody or antigen binding fragment that specifically binds to HIV-1 Env, or conjugate thereof (e.g. a conjugate including a detectable marker) under conditions sufficient to form an immune complex, and detecting the immune complex (e.g., by detecting a detectable marker conjugated to the antibody or antigen binding fragment).

In one implementation, the antibody or antigen binding fragment is directly labeled with a detectable marker. In another implementation, the antibody that binds HIV-1 Env (the primary antibody) is unlabeled and a secondary antibody or other molecule that can bind the primary antibody is utilized for detection. The secondary antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially. Suitable labels for the antibody, antigen binding fragment or secondary antibody are known and described above, and include various enzymes, prosthetic groups, fluorescent materials, luminescent materials, magnetic agents and radioactive materials.

In some implementations, the disclosed antibodies or antigen binding fragments thereof are used to test vaccines. For example, to test if a vaccine composition including an HIV-1 Env or fragment thereof assumes a conformation including the epitope of a disclosed antibody. Thus, provided herein is a method for testing a vaccine, wherein the method includes contacting a sample containing the vaccine, such as an HIV-1 Env immunogen, with a disclosed antibody or antigen binding fragment under conditions sufficient for formation of an immune complex, and detecting the immune complex, to detect the vaccine, such as an HIV-1 Env immunogen including the epitope, in the sample. In one example, the detection of the immune complex in the sample indicates that the vaccine component, such as an HIV-1 Env immunogen, assumes a conformation capable of binding the antibody or antigen binding fragment.

### III. Examples

The following examples are provided to illustrate particular features of certain implementations, but the scope of the claims should not be limited to those features exemplified.

### Example 1

### Materials and Methods

The following provides a description of materials and methods used in the examiner provided herein.

Expression of antibody variants in Expi293 cells. Antibody variable heavy chain and light chain sequences were codon optimized, synthesized and cloned into a VRC8400 (CMV/R expression vector)-based IgG1 vector as previously described(Kong et al., 2019). The variants were expressed by transient transfection in Expi293 cells (Thermo Fisher Scientific) using Turbo293 transfection reagent (SPEED BioSystems) according to the manufacturer's recommendation. 50 microgram plasmid encoding heavy-chain and 50 microgram plasmid encoding light-chain variant genes were mixed with the transfection reagents, added to 100 ml of cells at 2.5×10⁶/ml, and incubated in a shaker incubator at 120 rpm, 37° C., 9% CO₂. At 5 days post-transfection, cell culture supernatant was harvested and purified with a Protein A (GE Healthcare) column. The antibody was eluted using IgG Elution Buffer (Thermo Fisher) and were brought to neutral pH with 1 M Tris-HCl, pH 8.0. Eluted antibodies were dialyzed against PBS overnight and were confirmed by SDS-PAGE before use.

HEp-2 cell staining and Cardiolipin ELISA assay. Polyreactivity was determined by ANA HEp-2 Staining Analysis (ZEUS Scientific Cat. No: FA2400) and anticardiolipin ELISA (Inova Diagnostics Cat. No.: 708625). For the HEp-2 assay, all antibodies were tested at 25 and 50 μg/ml as per manufacturer's protocol and imaged on a Nikon Ts2R microscope for 500 ms. Scores from 0 to 3 were defined with four control antibodies VRC01-LS, 4E10. VRC07-523LS. and VRC07-G54W. Test antibodies were scored by visual estimation of staining intensity in comparison to the control antibodies. Scores equal to or greater than 1 at 25 μg/ml were classified as autoreactive, and between 0 and 1 as mildly polyreactive. In the cardiolipin ELISA, antibodies were tested at a starting concentration of 100 μg/ml, followed by 3-fold dilutions. IgG phospholipid (GPL) units were calculated from the standard curve. GPL score <20 was considered as not reactive, 20-80 as low positive and >80 as high positive.

Heparin affinity chromatography. Each antibody sample was diluted in 1500 μl of mobile phase A (MPA), 10 mM sodium phosphate, pH 7.2±0.2 to a final concentration of approximately 20 μg/mL. It was then injected onto the HiTrap 1 mL Heparin HP column (Cytiva Life Sciences, Marlborough, MA) on a BioRad (Hercules, CA) NGC Chromatography System Quest 10. The flow rate was set to 1.0 mL/min and the mobile phase B (MPB) was 10 mM sodium phosphate, 1 M NaCl, pH 7.2±0.2. The column was equilibrated in 100% MPA before each injection; the gradient was (1): 0-2 min, 100% MPA; (2): 2-12 min, 100% MPA to 100% MPB; (3) 12-14 min, 100% MPB. UV absorbance was detected at 280 nm using Chromlab.

Neutralization assay. Single-round-of-replication Env pseudoviruses were prepared, titers were determined, and the pseudoviruses were used to infect TZM-bl target cells as described previously (Sarzotti-Kelsoe et al., 2014). Neutralization of monoclonal antibodies was determined using a multiclade panel of 12 HIV-1 Env-pseudoviruses including clade A (2), clade AG (1), clade B (4), clade C (4), and clade D (1), and using a 208-isolate panel (Doria-Rose et al., 2012). Each mAb was assayed at 5-fold dilutions starting at 50 g/ml. The neutralization titers were calculated as a reduction in luminescence units compared with control wells and reported as 50% or 80% inhibitory concentration (IC₅₀ or IC8o) in micrograms per milliliter.

Human FcRn knock-in and human FcRn-hFc (Tg32-hFc) knock-in mouse pharmacokinetics. Human FcRn transgenic mice (C57BL/6, B6.mFcRn−/− hFcRn Tg32 line from The Jackson laboratory) and human FcRn-hFc mice (Tg32-hFc line from The Jackson laboratory) were used to assess the pharmacokinetics of VRC07-523LS and N6LS antibody variants. Each animal was infused intravenously with 5 mg of mAb/kg of body weight. Whole blood samples were collected at day 1, 2, 5, 7, 9, 14, 21, 28, 35, 42, and 56. Serum was separated by centrifugation. Serum mAb levels were measured by ELISA as described previously (Rudicell et al., 2014). All mice were bred and maintained under pathogen-free conditions at an American Association for the Accreditation of Laboratory Animal Care (AAALAC)-accredited animal facility at the NIAID and housed in accordance with the procedures outlined in the Guide for the Care and Use of Laboratory Animals. All mice were between 6 and 13 weeks of age. The study protocol was evaluated and approved by the NIH Animal Care and Use Committee (ASP VRC-18-747). The pharmacokinetic parameters were calculated using the Phoenix WinNonlin software (Certara). Two different parameters for half-life and the clearance rates were calculated: using i) data sets at day 42 post-infusion with no ADA responses to mAbs and ii) all data sets at day 42 post-infusion, including ones from animals that developed ADA responses to mAbs before day 42. To calculate the area under the curve of the serum mAb concentration versus time profile, data sets at day 42 post-infusion with no ADA responses were used.

Amino Acids frequency analysis. HIV antibody sequences were downloaded from GenBank on Aug. 7, 2021, and kept the sequences identified as Homo sapiens for amino acids frequency analysis. The Kabat numbering was assigned to the sequences using standalone ANARCI (Bioinformatics. 2016 Jan. 15; 32(2):298-300). The sequences that could not be assigned Kabat numbering were removed from analysis. An in-house python script was applied to calculate the frequency of amino acids from 6,589 heavy chains and 7,109 light chains of HIV antibodies. We used AbYsis server to obtain the amino acids frequency (J Mol Biol. 2017 Feb. 3; 429 (3):356-364.) of human antibodies (http://www.abysis.org/abysis/searches/distributions/distributions.cgi).

Fc-FcRn binding kinetics by surface plasmon resonance. The binding kinetics of VRC07-523LS variants to FcRn was measure using Biacore T200 (Cytiva). The FcRn/β2m heterodimers in HBS-EP+ buffer (10 mM HEPES, 7.4, 150 mM NaCl, 3 mM EDTA, 0.005% v/v Surfactant P20) in a two-fold dilution series from the highest concentration of 1 to 0 M were passed over the VRC07-523LS variants captured (-300 RU) by HIV-1 gp120 coreₑ clade A/E 93TH057 (Kwon et al. PNAS) immobilized onto a CM5 chip by amine coupling and monitored association for 120 seconds at a flow rate of 40 μl/min and dissociation for 60 seconds. In another format, VRC07-523LS variants in HBS-EP+buffer in a two-fold dilution series from the highest concentration of 500 nM were passed over the biotinylated FcRn/β2m heterodimer captured (˜150 RU) on an SA chip at a flow rate of 40 μl/min. and monitored association for 120 sec. and dissociation for 120 seconds. The kinetics parameters were extracted by fitting the sensograms with 1:1 Langmuir model using BIA evaluation software.

Fc-FcRn binding kinetics by bio-layer interferometry. We used Octet HTX system (ForteBio) to measure the Fc-FcRn binding kinetics with FcRn/2m as ligand. Ni-NTA biosensors were soaked in PBS for 10 min, loaded the His-tagged FcRn/β2m the density of ˜0.3 nm by immersing the biosensors in the wells containing 125 nM of FcRn/β2m for 1 min, baselined for 1 min at HBS-EP+buffer (GE Healthcare), and measured association by dipping the biosensors into the wells containing VRC07-523LS variants in series of concentrations ranging from 0 to 1000 nM in HBS-EP+buffer for 3 min and dissociation at HBS-EP+buffer for 1 min. For reference, we repeated the entire steps mentioned above but with blank Ni-NTA biosensors. Binding kinetic sensograms were globally fitted with 1:1 model and double referencing to extract kinetic parameters.

Data analysis. Accessible surface area (ASA) was calculated using PDBePISA (Krissinel and Henrick, 2007). Distances of amino acids from respective epitopes were calculated using PyMOL(The PyMOL Molecular Graphics System, Version 2.0 Schradinger, LLC). Net charge and the isoelectric point (pI) of variants were calculated using EMBOSS Pepstats (Rice et al., 2000) at ebi.ac.uk/Tools/seqstats/emboss_pepstats/. Unpaired t-tests were calculated using GraphPad Prism version 8 for Windows (GraphPad Software, La Jolla California USA, www.graphpad.com).

### Example 2

### Broad HIV-Neutralizing VRC01-Class Antibodies with Enhanced Pharmacokinetics Achieved by Reduction of Net Positive Charge on the Variable Domain

### Abstract

The amino-acid composition of the immunoglobulin G variable region has been observed to impact antibody half-lives. With VRC01-class antibodies, for example, the insertion of a framework region 3 loop decreases polyreactivity and enhances half-life. This example provides results showing that the improved pharmacokinetics (PK) of VRC01-class antibodies by framework region 3 loop insertion correlated with the incorporation of a patch of acidic amino acids and applied the correlation between improved PK and reduced positive charge to increase in vivo half-lives of VRC01-class antibodies, VRC07-523LS and N6LS. We used a structure-based approach to generate a panel of antibody variants with select Arg or Lys in framework regions mutated to Asp, Gln, Glu, or Ser. vVRC07-523LS and N6LS variants incorporating these mutations exhibited reduced affinity to heparin, which closely correlated with the reduced polyreactivity and improved PK in human FcRn knock-in mice. In particular, VRC07-523LS.v11, v14, v26, v32 and v34 incorporating Arg or Lys to Asp, Glu, or Ser mutations exhibited improved PK, of which VRC07-523LS.v34 showed improved in vivo half-life of 10.1 days from 6.4 days, and neutralized 91.8% of 208 viruses at IC8o <1 μg/ml, while VRC01LS neutralized only ˜45%. The improved half-lives of these variants assessed in human FcRn knock-in mice were similar to or slightly better than the half-life of VRC01LS, which has a half-life of 71 day in humans. Other VRC01-class antibody variants, N6LS.C1, C15, C30, and C35 incorporating Arg or Lys to Asp or Glu mutations also showed superior PK in human FcRn mice compared to VRC01LS, and neutralized >73% of 208 viruses at IC80<1 μg/. Furthermore, Arg and Lys resides were observed to be prevalent at select positions in human antibodies, and we propose that mutating these to negatively charged residues may be a general means to improve PK. Thus, the substitution of select Arg or Lys with Asp, Gln, Glu, or Ser in the framework region of VRC01-class antibodies can increase in vivo PK parameters.

### Introduction

Passive transfer of highly potent and broadly reactive HIV-1 neutralizing antibodies also holds promise to prevent HIV-1 infection (Corey et al., 2021; Gruell and Klein, 2018; Julg and Barouch, 2019), as an alternative to a vaccine. Antibody-mediated prevention, however, requires frequent infusion of large antibody doses and is costly, prompting the development of antibodies with prolonged in vivo half-lives to make this approach more feasible and affordable. Efforts to extend the in vivo half-life of antibodies have focused on enhancing pH-dependent antibody Fc and the neonatal Fc receptor (FcRn) interactions, some of which include, LS, YTE, and DHS mutations that yield substantially improved serum half-lives (Dall'Acqua et al., 2006; Grevys et al., 2015; Hinton et al., 2004; Lee et al., 2019; Mackness et al., 2019; Ward et al., 2015; Zalevsky et al., 2010). However, these mutations have not been effective for all antibodies. Moreover, antibodies with identical Fc domains can have different levels of improvement in their half-lives and clearance rates, indicating that the Fab region also contributes to antibody homeostasis (Piche-Nicholas et al., 2018; Schlothauer et al., 2013; Schoch et al., 2015; Suzuki et al., 2010; Wang et al., 2011). It has also been reported that antigen binding alters dynamics of antibody-FcRn interaction to differentially affect the in vivo half-lives and clearance rates of antibodies (Sun et al., 2020). Other factors associated with the serum half-lives and clearance rates involved physical characteristics of antibodies, such as their solubility, thermal stability, polyreactivity, and off-target binding mediated by hydrophobic or charge-charge interactions (Boswell et al., 2010; Sigounas et al., 1994; Xu et al., 2019). Indeed, improved potency and breadth of antibodies by engineering or library-based screenings are often accompanied with enhanced polyreactivity, which in turn results in a fast clearance rate and reduced serum half-life (Pepinsky et al., 2010; Rudicell et al., 2014; Sievers et al., 2015; Wu et al., 2007).

In terms of VRC01-class antibodies, although the increased hydrophobicity of VRC07-523 and NIH45-46 with G54W mutation resulted in increased potency, at the same time it led to the increased polyreactivity and reduced in vivo half-lives (Diskin et al., 2011; Rudicell et al., 2014).

Charge-mediated interactions are also associated with polyreactivity and clearance rates as antibodies with net positive charge at physiological pH interact with negatively charged endothelial cell membranes, which leads to the increased antibody absorption by endocytosis (Bernfield et al., 1999; Kraft et al., 2020)-(Boswell et al., 2010). Taken together, IgG serum turnover has associated not only with Fc-FcRn interaction, but with off-target binding interactions between the Fab regions of antibodies and vesicular cell membranes mediated by hydrophobic or electrostatic interactions, triggering elevated polyreactivity and fast clearance from the blood steam.

We engineered VRC01-class antibodies with improved serum half-lives by reducing off-target interactions mediated by charge-charge interaction while maintaining high potency and breadth. We investigate the basis for reduced autoreactivity and increased half-lives of VRC01-class antibodies by 03FR3 loop insertion, in which four aspartates introduced by 03FR3 loop insertion appear to be responsible for reduced polyreactivity and increased half-life. We also observed reduced affinity of antibody to heparin to correlate with the reduced autoreactivity. We applied these findings to improve the half-lives of antibodies by generating a panel of antibody variants with select Arg or Lys to Asp, Gln, Glu, or Ser mutations, testing polyreactivity, neutralization potency, affinity to heparin, and assessing in vivo half-lives of antibody variants in human FcRn knock-in mice.

### Results

### Basis of Reduced Autoreactivity by VRC03 Framework Region 3 Loop Insertion.

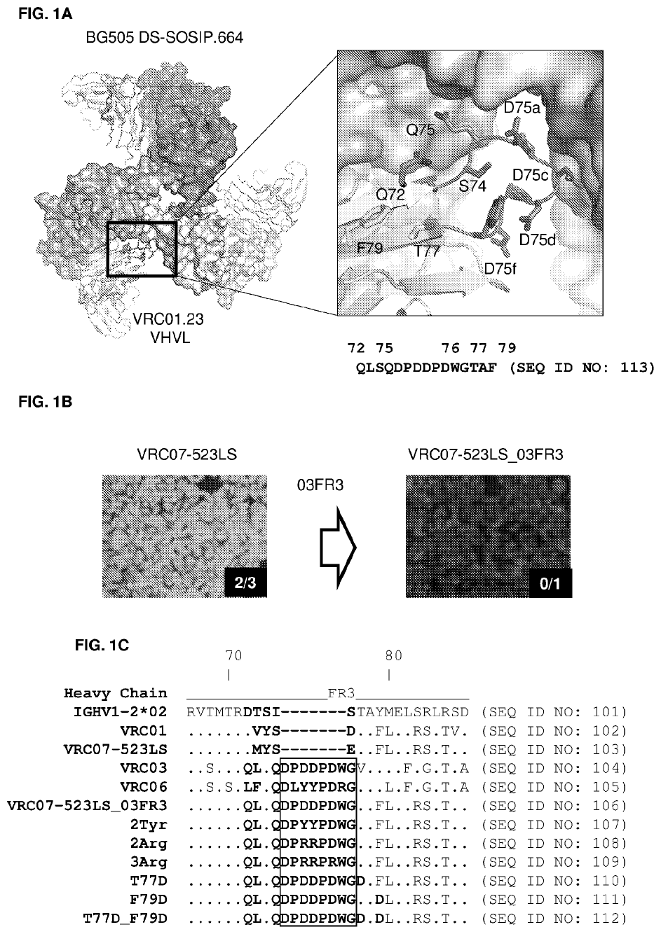

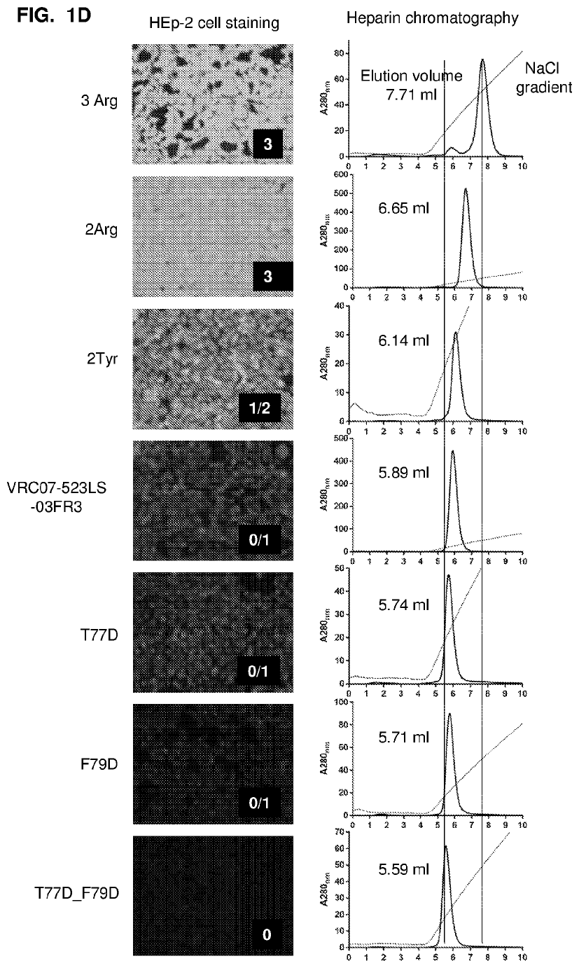

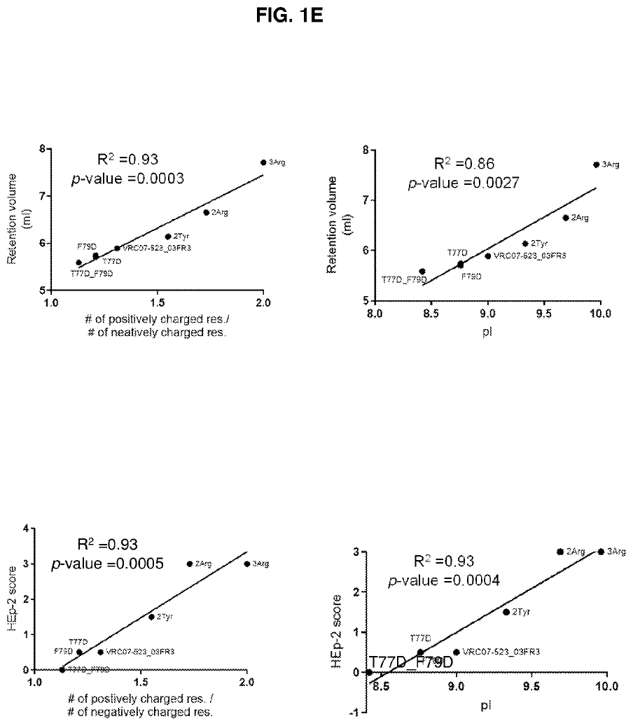

Previously, we reported 03FR3 loop insertion to VRC01-class antibodies reduces autoreactivity and improves potency by enabling the antibodies to engage the CD4-binding site-2 (CD4BS-2) in a neighboring Env protomer more effectively (Liu et al., 2019). However, the basis for how this extended loop insertion reduced polyreactivity was not clear. We noticed, however, that the loops insertion comprised four aspartic acid residues in the seven-residue insert (FIGS. 1A and 1B). One explanation is that the reduced net positive charge due to the presence of four aspartic acid residues reduced autoreactivity by decreasing charge mediated non-specific interactions between the antibody and the vascular endothelial membranes at physiological pH. To test this, we generated a panel of VRC07-523LS_03FR3 variants comprised of two distinct groups: one group with more positively charged variants and the other with less positively charged variants than VRC07-523LS_03FR3 (FIG. 1C). The former group included: i) VRC07-523LS_03FR3_2Tyr where the Asp75c and Asp75d were replaced with Tyr, the counterpart of VRC06, ii) VRC07-523LS_03FR3_2Arg where Asp75c and Asp75d were replaced with Arg, and iii) VRC07-523LS_03FR3_3Arg where Asp75c, Asp75d, and Asp75f were replaced with Arg. The latter group included, iv) VRC07-523LS_03FR3_T77D where Thr77 was replaced with Asp, v) VRC07-523LS_03FR3_F79D where Phe79 was replaced with Asp, and vi) VRC07-523LS_03FR3_T77D_F79D where Thr77 and Phe79 were replaced with Asp.

Variants i)-iii) were generated to exhibit increased autoreactivity while variants iv)-vi) were expected to show further reduced autoreactivity than VRC07-523LS_03FR3 (FIG. 1C). HEp-2 cell binding assays on these variants exhibited exactly what we anticipated. Variants i)-iii) comprising more positively charged mutations than VRC07-523LS_03FR3 showed increased autoreactivity in the rank order of 3Arg>2Arg>2Tyr, while variants iv-vi) all showed further reduced autoreactivity and reduced affinity, demonstrating that the reduced autoreactivity attributes to the reduced net positive charge due to 03FR3 loop insertion (FIG. 1D). Affinity to heparin significantly correlated with autoreactivity assessed in HEp-2 cell binding assay (FIG. 1D). Thus, reduction in net positive charge either by replacing Arg to amino acids with polar or aromatic side chains or by introducing more Asp directly associated with reduced affinity to heparin and the reduced autoreactivity (FIGS. 1D and E).

### Workflow for Surface Charge Engineering to Improve Pharmacokinetics of HIV-1 Antibodies

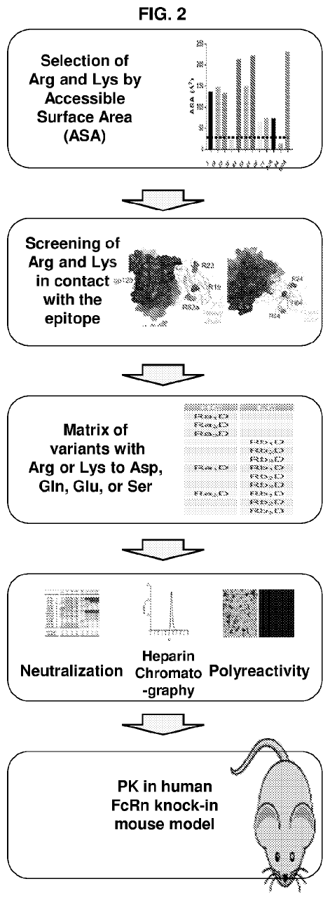

To select Arg and Lys residues for substitution, we first calculated the accessible surface area (ASA) of all Arg and Lys residues within the variable domain, to eliminate those with low ASA, and then screened the remaining Arg and Lys residues for those that make direct contacts with epitopes or reside within 5 Å from an epitope or make contacts with a neighboring protomer to remove them from the list (FIG. 2). The final selection of Arg and Lys in the heavy and light chain were, then, substituted with Asp, Gln, Glu, or Ser and variants incorporating these single mutations were evaluated for their neutralization potency in a 12-representative isolate panel and were also assessed for their affinity to bind heparin. Single mutations that reduced affinity to heparin and retained or reduced potency by less than or approximately 2-fold were selected and used to generate a panel of variants comprised of single mutation combinations in heavy and light chains. Variants with the best scores in neutralization potency, affinity to heparin, and autoreactivity were selected and assessed for improved PK in human FcRn knock-in mice (FIG. 2).

A Panel of VRC07-523LS Surface Charge Antibody Variants with Arg or Lys to Asp, Glu, Ser, or Gln Mutations in the Variable Domain Identified Variants with Improved In Vivo Half-Life

### 1^{st} Round of Screening

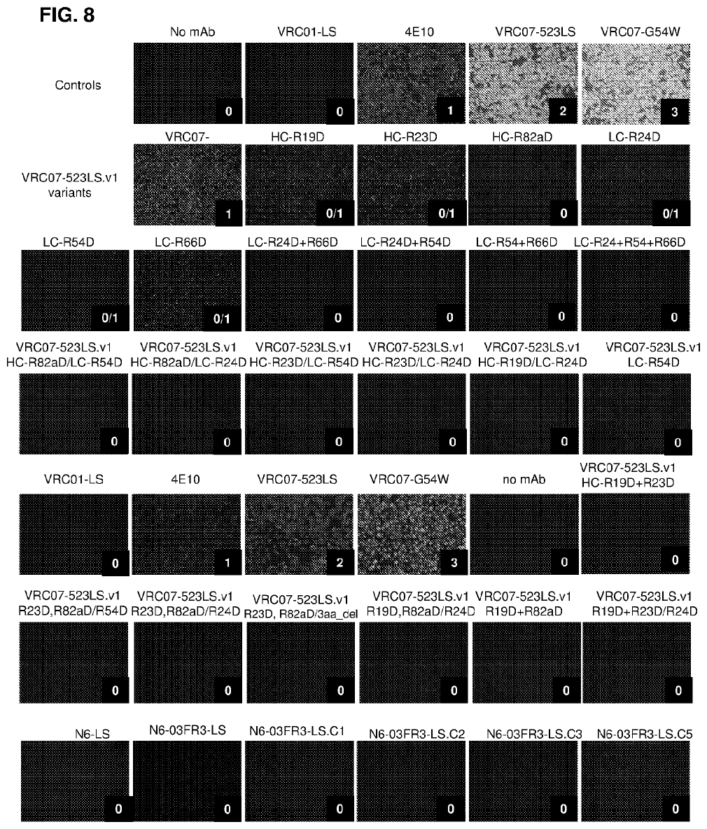

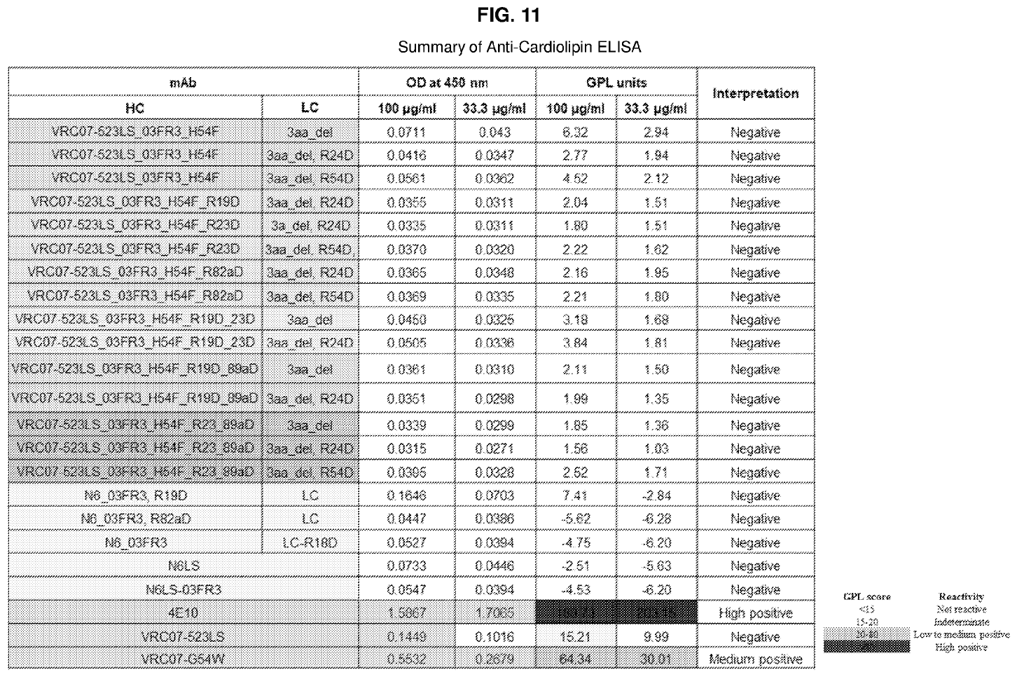

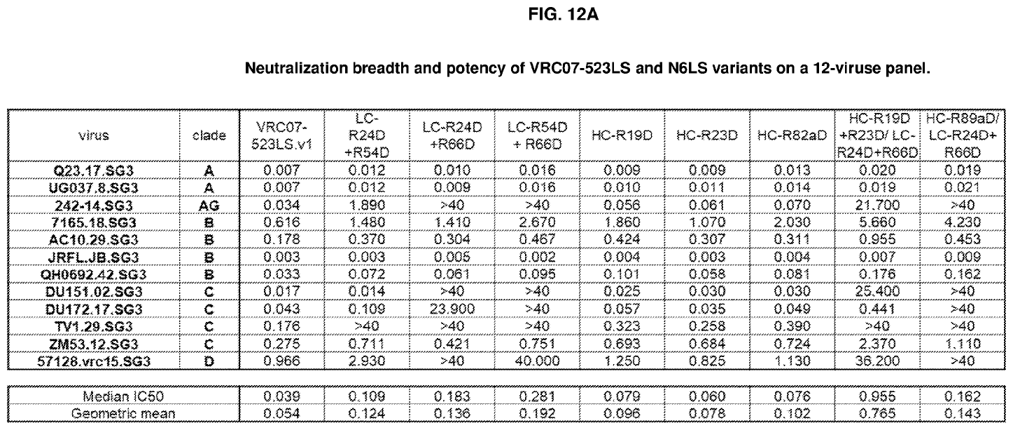

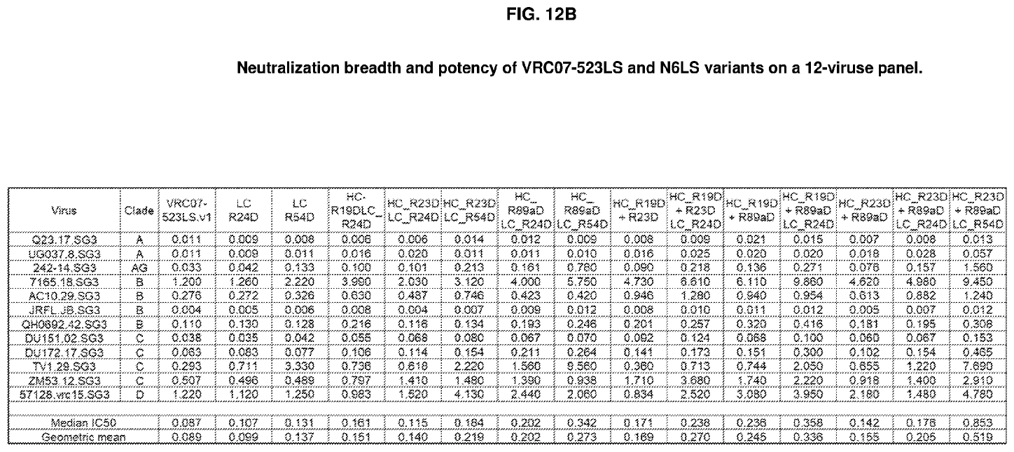

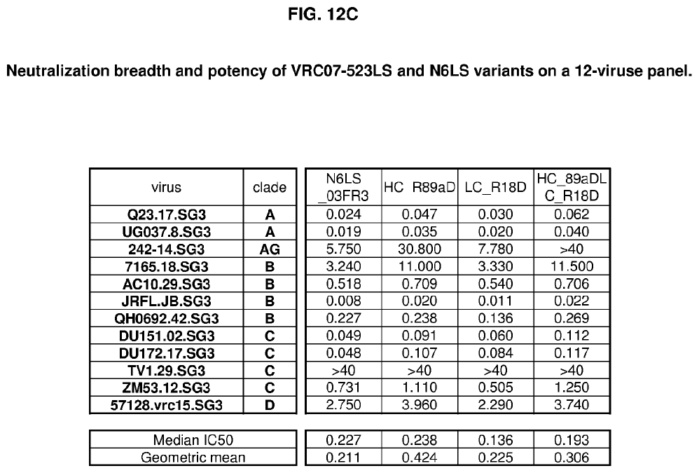

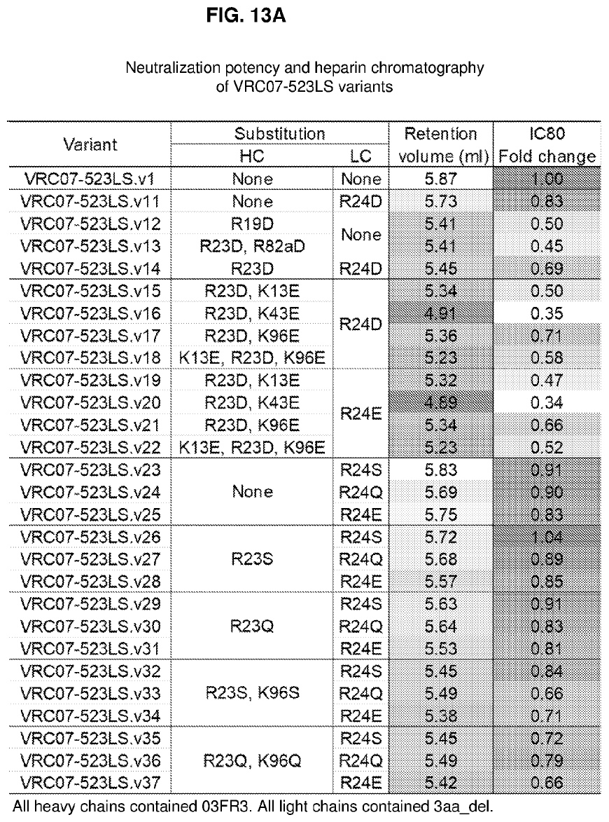

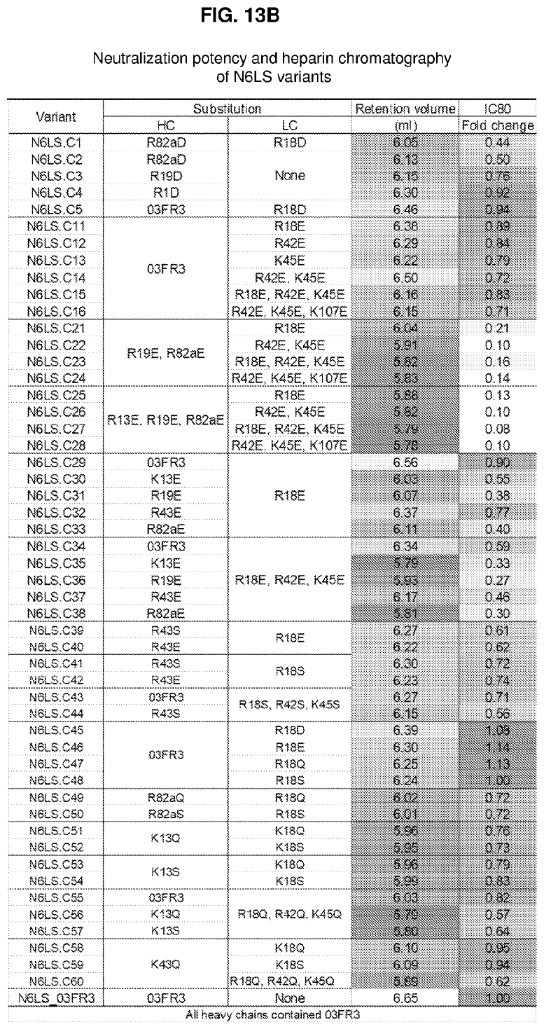

To improve the half-life of VRC07-523LS.v1 by reducing net positive charge in the variable domain, we first focused on 12 Arg and 6 Arg in the heavy and light chain, respectively. Arg19, Arg23, and Arg82a in the heavy chain and Arg24, Arg54, and Arg66 in the light chain were selected for testing after removing Arg residues in contacts with the epitope or within 5 Å from the epitope or those buried substantially (FIG. 3A). A panel of antibody variants was generated incorporating single mutations or double mutations in the heavy and light chains, and in combinations of heavy and light chain mutations, assessed autoreactivity against HEp-2 cell and cardiolipin binding, and neutralization potency in a 12-virus panel (FIG. 8 and FIGS. 11 and 12). We found that all resultant variants reduced autoreactivity and no autoreactivity was observed for variants incorporating double mutations. Each single heavy chain mutation preserved potency, but double mutations in the light chain, particularly, variants containing the light chain Arg66 to Asp mutation (R66D) substantially reduced potency (FIG. 12), which we discovered from the cryo-EM structure of VRC01.23-bound BG505 DS-SOSIP that Arg66 in the light chain to be critical for VRC01 and VRC07 function because Arg66 interacts via a salt bridge with the glycan at HIV-1 Env gp120 residue 276 (the right panel of FIG. 3A). Therefore, we generated a panel of variants comprising Arg19, Arg23, or Arg82a to Asp mutation in the heavy chain and Arg24 or Arg54 to Asp mutation in the light chain (FIG. 12). Each of these variants showed the potencies reduced less than 2-fold compared to the parental, except for triple mutations which showed potencies reduced more than 3-fold, particularly, for variants with an Arg54 to Asp change in the light chain (FIG. 12). Eventually, we selected and assessed VRC07-523LS.v11, v12, v13, and v14 for PK in human FcRn knock-in mouse model, as these four variants showed reduced affinity to heparin and the potencies comparable to the parental VRC07-523LS.v1 (FIG. 3B). All four variants showed improved half-lives, area under the curve (AUC), and reduced clearance rates, of which VRC07-523LS.v11 and v14 showed half-lives improved to 11 days and 8.4 days from 6 days, area under the curve to 442 [day*(μg/ml)] and 329 from 155 and reduced clearance rates to 11.4 (ml/day/kg) and 15.5 from 32.5 at days 42 post-infusion, respectively. (FIGS. 3C and D). [Two different groups of parameters were calculated; i) using all animals at day 42 post-infusion including animals that have developed anti-drug antibody (ADA) responses and ii) animals with no ADA response at day 42 post-infusion only.](FIG. 3D). However, VRC07-523LS.v12 and VRC07-523LS.v13 did not show as much improvement in AUC and clearance rates as VRC07-523LS.v11 and VRC07-523LS.v14. VRC07523LS.v12 and VRC07-523LS.v13 variants also showed their potencies more reduced than v11 and v14 variants. 2^{nd} round of screening With these findings, we next sought to improve VRC07-523LS.v11 and VRC07-523LS.v14 further by reducing their affinity to heparin while maintaining the potency, and tested variants incorporating Lys13, Lys43, or Lys96 to Glu mutation in the heavy chain (FIG. 3A and FIG. 12A). Adding K43E to VRC07-523LS.v14 (which becomes VRC07-523LS.v16) reduced its affinity to heparin the most followed by addition of K13E (VRC07-523LS.v15) and by K96E (VRC07-523LS.v17), as shown by the shift of the retention volume of variants on heparin chromatography from 5.12 ml for VRC07-523LS.v14 to 4.6 ml, 5.0 ml, and 5.02 ml, respectively (FIG. 13A).

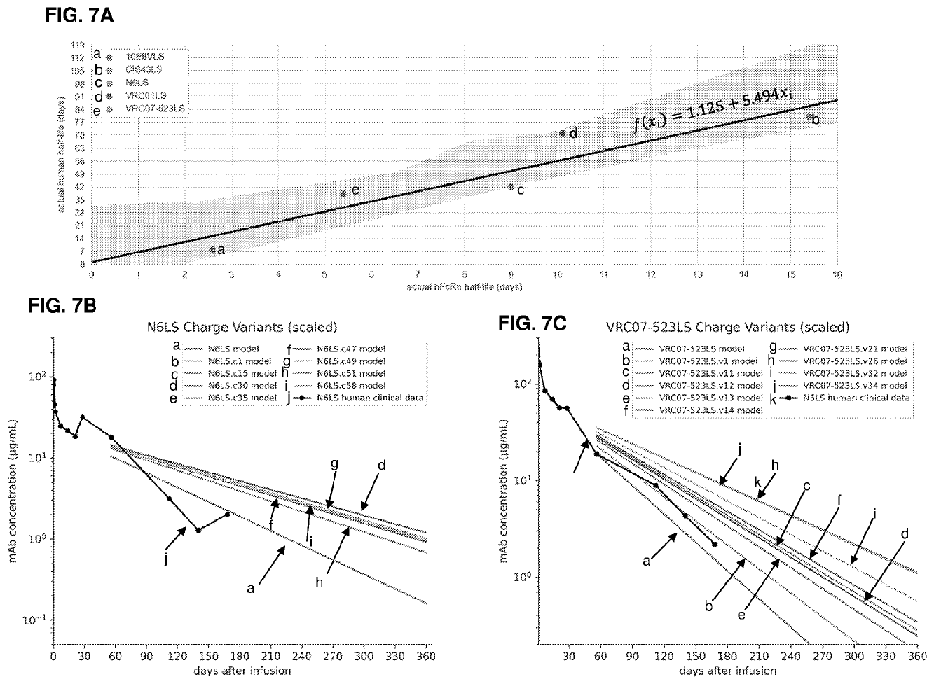

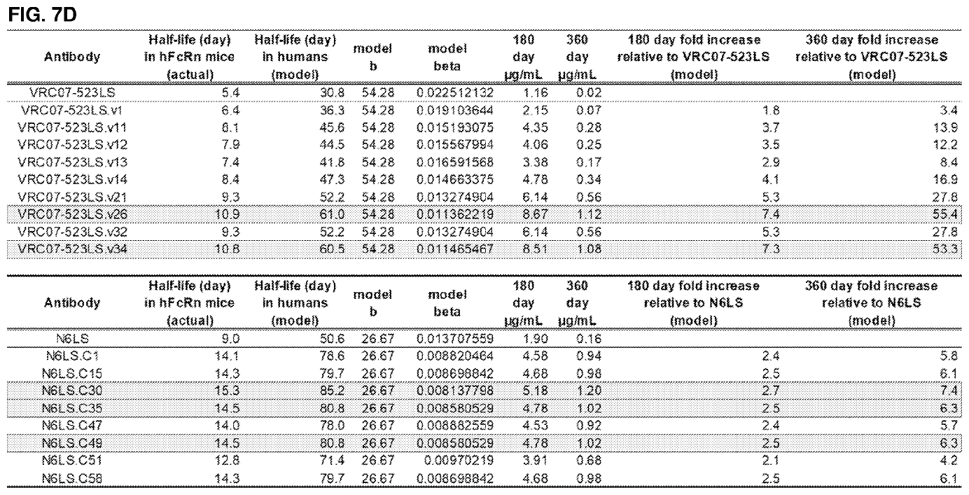

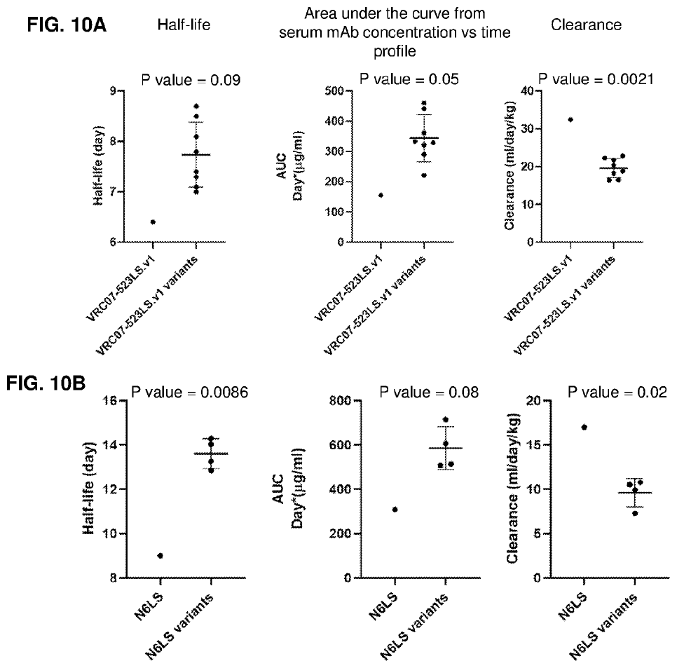

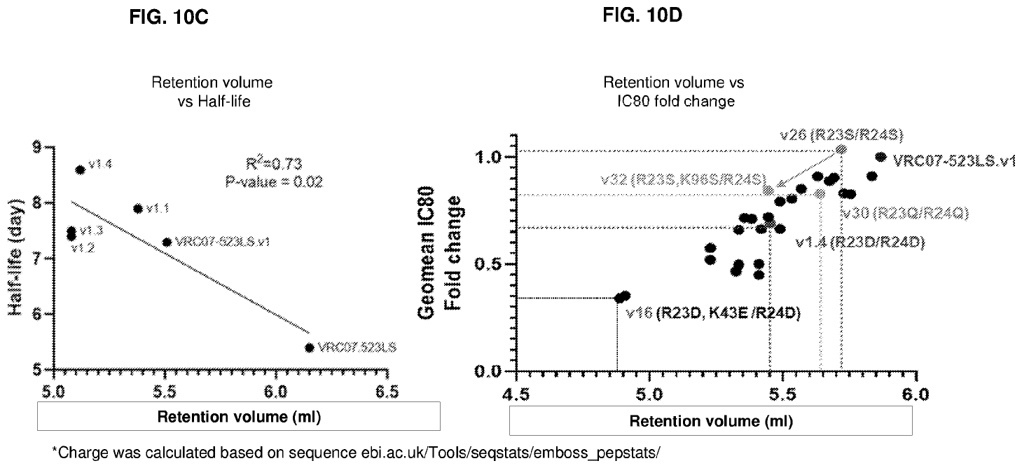

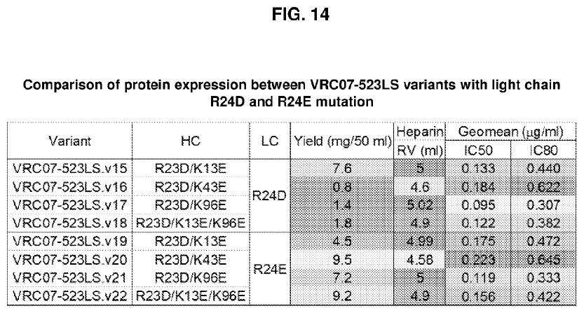

To compare the effect of altering Arg24 to Asp vs Glu mutation in the light chain, Asp24 in the light chain of the above-mentioned variants (VRC07-523LS.v15-VRC07-523LS.v18) were replaced with Glu. The four variants with R24E in the light chain (VRC07-523LS.v19-VRC07-523LS.v22) showed the same trend toward affinity to heparin and the potency as in variants with R24D but with slightly elevated effects on affinity to heparin and the potency than the variants with R24D mutation (FIG. 13A). Interestingly, variants with R24E mutation in the light chain showed better expression yields in transient transfection than their counterparts with R24D in the light chain (FIG. 14). Therefore, we selected VRC07-523LS.v21 for PK assessment over VRC07-523LS.v17 as VRC07-523LS.v21 variant showed the similar properties on affinity to heparin and IC80 fold changes, but with better yields of variants. In human FcRn mouse PK study, VRC07-523LS.v21 showed PK parameters similar to VRC07-523LS.v14, but with slightly increased half-life compared to VRC07-523LS.v14 (FIGS. 3C and 3D). Thus, the results led us to focus on Arg23 and Lys96 in the heavy chain and Arg24 in the light chain for further testing, as variants incorporating mutations on these residues showed reduced affinity to heparin while maintaining the potency (FIG. 12A). 3′^{d} round of screening Next, to explore effects of mutations other than Asp or Glu, we generated another panel of antibody variants containing Arg23HC, Lys96HC, and Arg24_{LC} replaced with uncharged polar amino acids, Ser or Gln, compared them with Glu mutation, and found the overall rank order of Ser >Gln>Glu mutation for potency from high to low and Glu>Gln>Ser mutation for affinity to the heparin from low to high by a slight margin (FIG. 13A). All these 15 variants, VRC07-523LS.v23 to VRC07-523LS.v37, maintained the potency or exhibited the potency less than 2-fold reduced compared to that of parental VRC07-523LS.v1, while exhibiting reduced affinity to heparin. We selected VRC07-523LS.v26, VRC07-523LS.v32, and VRC07-523LS.v34 for PK assessment as VRC07-523LS.v26 maintained the potency with modest decrease in affinity to heparin (FIGS. 7, 13A), and VRC07-523LS.v32 and VRC07-523LS.v34 variants exhibited the most reduced affinity to heparin with potencies comparable to the parental (FIG. 3B and FIG. 13A). The PK of these three variants assessed in human FcRn mouse was inversely correlated with their affinities to heparin (FIGS. 7, 10C). VRC07-523LS.v34, which showed the lowest affinity to heparin, exhibited the most improved AUC of 461 [day*(pg/ml)](FIGS. 3C and 3D).

In summary, VRC07-523LS variants, VRC07-523LS.v11, VRC07-523LS.v14, VRC07-523LS.v26, VRC07-523LS.v32, and VRC07-523LS.v34, selected based on their binding affinity to heparin and neutralization potency exhibited improved PK (FIGS. 3C and 3D) with their potencies comparable to the parental VRC07-523LS.v1; VRC07-523LS.v11, v14, v32, and v34 neutralized 92% of the 208-virus panel with an IC80 less than 1 μg/ml, respectively (FIGS. 3E and 3F). Among these, VRC07-523LS.v34, which showed the lowest affinity to heparin and the potency comparable to that of VRC07-523LS.v1, exhibited the most improved K parameters: half-life of 10.1 days, AUC of 471 [day*(pg/ml)], and the clearance rate of 11.3 ml/day/kg, from 6.4, 155, and 32.4, respectively (FIGS. 3C and D).

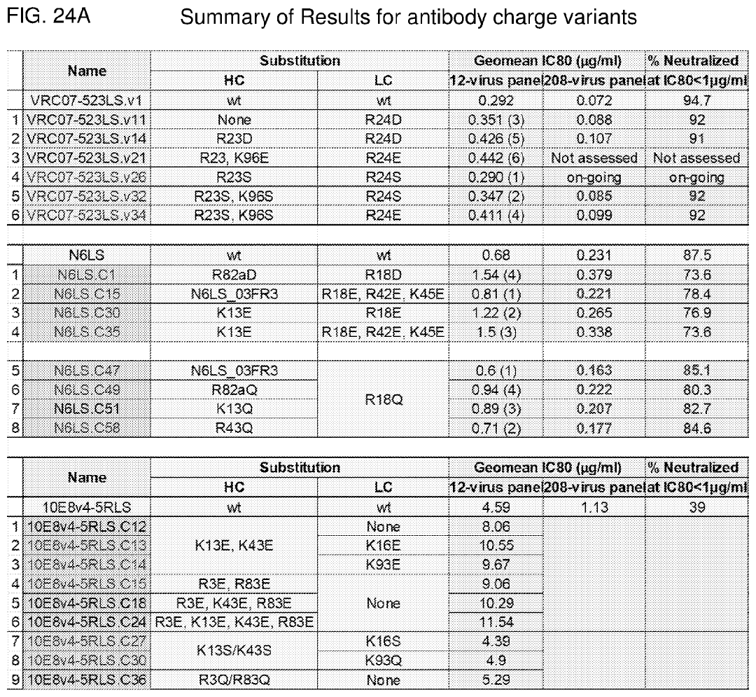

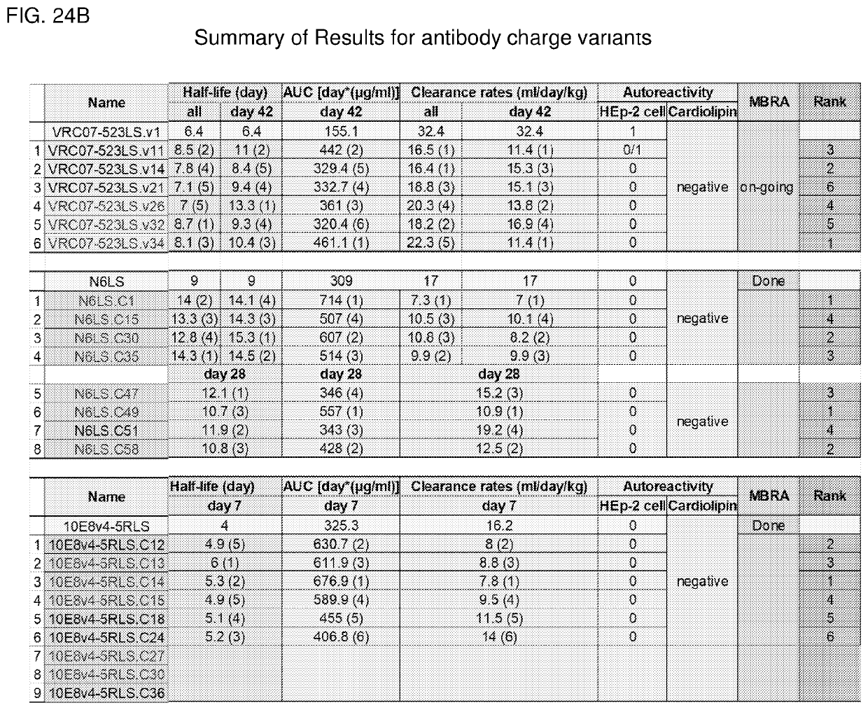

A summary of HIV-1 neutralization, heparin binding, and PK data for several variant VRC07-523LS antibodies is provided in FIGS. 24A and 24B.

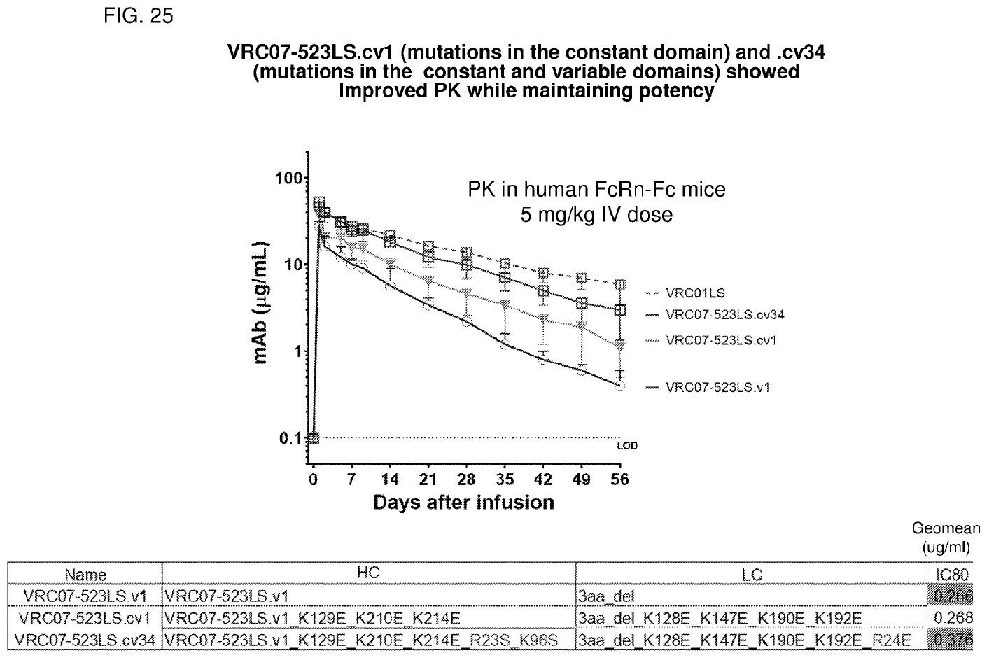

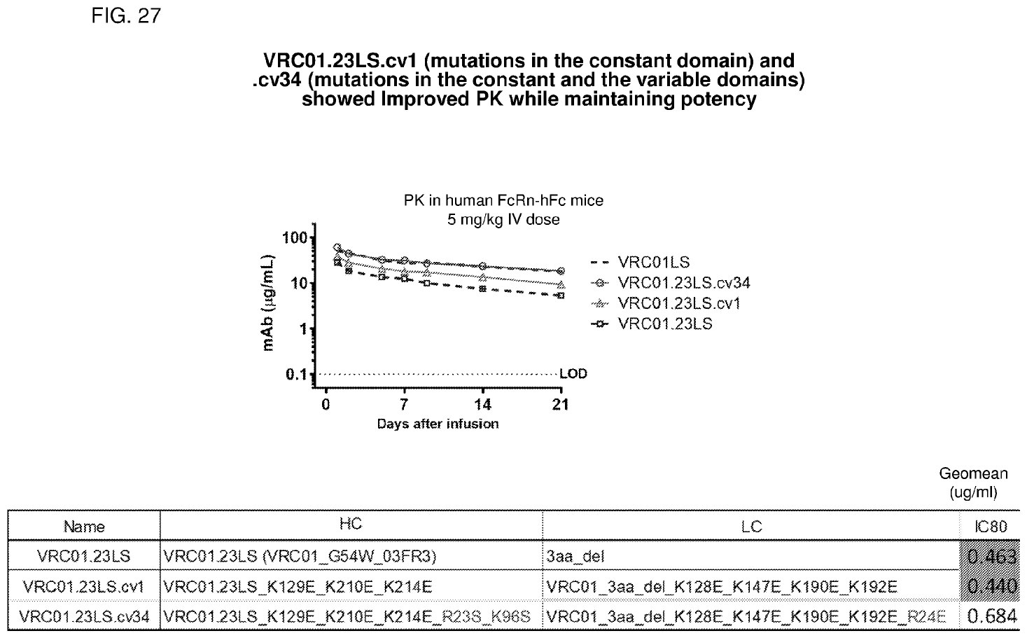

The VRC07-523LS antibody was also modified with mutations in the heavy and light chain constant domains and assessed for virus neutralization and pharmacokinetics (FIG. 25). The mutations included K129E, K210E, and K214E in the heavy chain constant domain and K128E, K147E, K190E, and K192E in the light chain constant domain. These were also assessed with the addition of the v34 mutations to the variable regions, R23S and K96S in the heavy chain variable region and R24E in the light chain variable region. These mutations were also assessed in the context of the VRC01.23LS antibody (FIG. 27)

A Panel of N6LS Surface Charge Antibody Variants Incorporating Arg or Lys to Asp or Glu Mutations in the Variable Domains Identified Variants with Improved In Vivo Half-Life

### 1^{st} Round of Screening

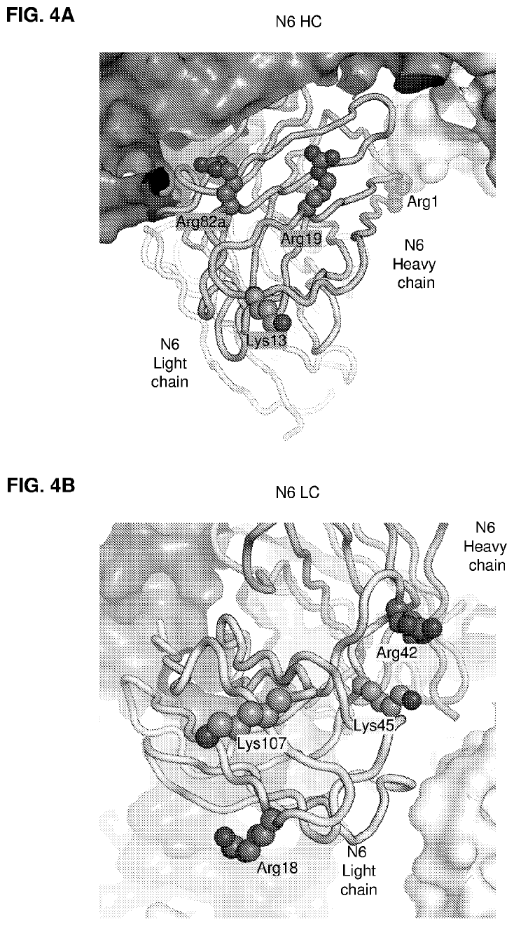

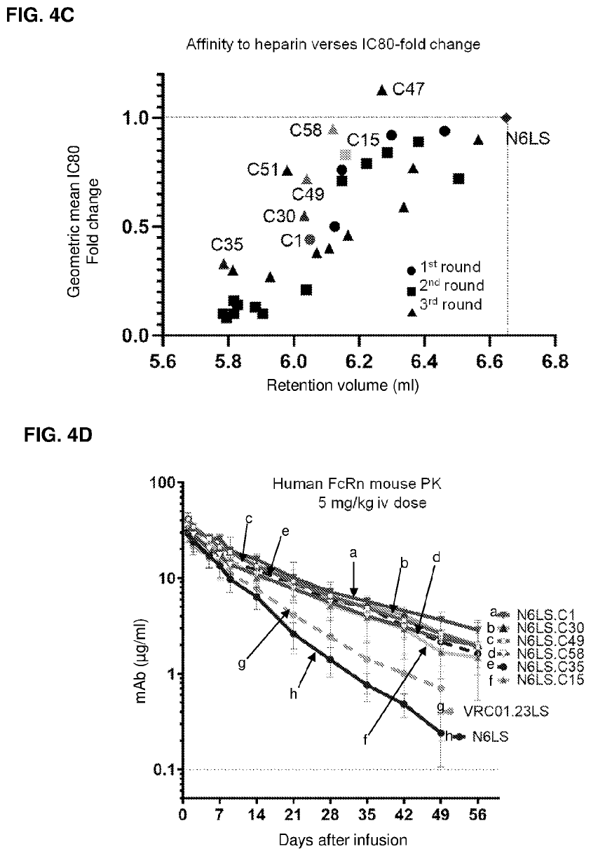

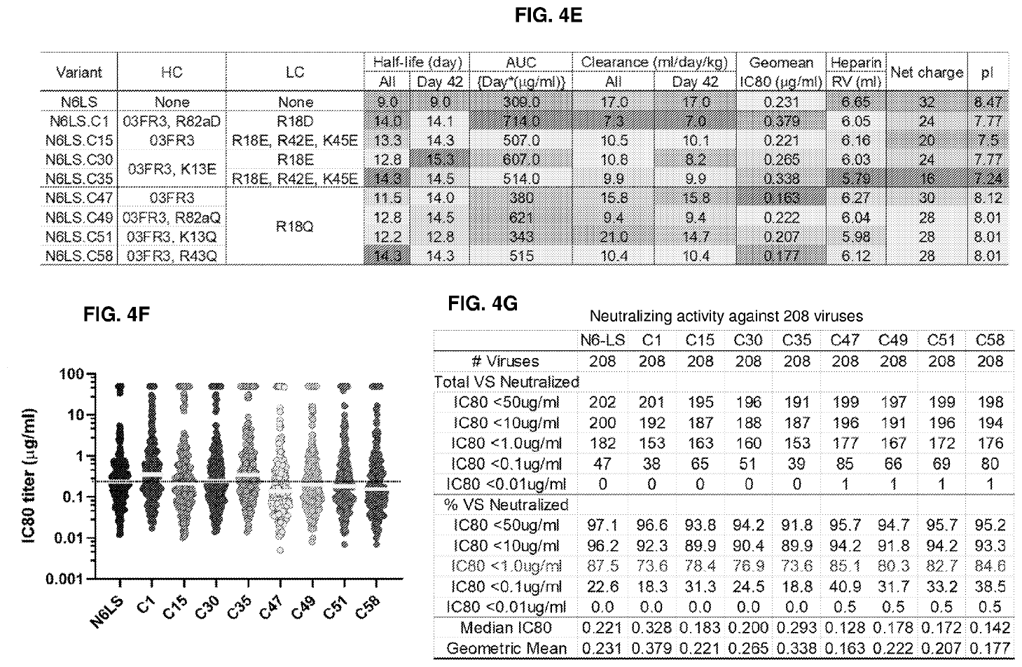

Inspired by VRC07-523LS charge variants with improved in vivo half-lives, we engineered N6 variants for reduced affinity to heparin with neutralization potency maintained. First, we selected Arg1, Arg19, and Arg82a in the heavy chain and Arg18 in the light chain for Asp substitution, as these residues are highly exposed and do not contact with epitopes (FIGS. 4A and B). On a 12-strain HIV-1 pseudovirus panel, N6LS variants incorporating one of select Arg to Asp mutations in the heavy or/and light chain, N6LS.C1 through N6LS.C5, exhibited their neutralization potencies which were reduced less than 2-fold compared to the parental N6LS (FIG. 13B). Of these N6LS.C1, which incorporated an Arg82a to Asp mutation in the heavy chain and Arg18 to Asp mutation in the light chain showed the greatest reduction in affinity to heparin (FIG. 4C, FIG. 13B). Therefore, we assessed the PK properties of N6LS.C1 in the human FcRn knock-in mouse model and found its half-life increased to 14 days from 9 days, the area under the curve (AUC) of the mAb concentration vs time profile increased to 714 from 309 [days*(μg/ml)], and its clearance rates decreased to 7 from −17 ml/day/kg, compared to that of its parental, N6LS (FIGS. 4D and E).

### 2^{nd} Round of Screening

Next, we extended our screen by testing N6LS.C1 variants incorporating a Lys13 to Glu mutation in the heavy chain, and Lys42, Arg45, or Lys107 to Glu mutation in the light chain (FIG. 13B). Notably, the affinities of N6LS variants to heparin and their neutralization potencies were more substantially impacted by the charge reversing mutations in the heavy chain than those in the light chain. N6LS.C21 through N6LS.24 which incorporated the double mutations, R19E and R82aE, in the heavy chain, and N6LS.C25 through N6LS.C28 which incorporated the triple substitutions, K13E, R19E, and R82aE, in the heavy chain all showed substantially reduced neutralization potency compared to a group of variants, N6LS.C11 through N6LS.C16, which contained only light chain mutations (FIG. 13B). On the contrary, N6LS.C15 and N6LS.C16 incorporating three mutation in the light chain maintained potency with reduced affinity to heparin. N6LS.C15 and showed improved PK parameters: half-life, AUC, and the clearance rate of 13.2 days, 507 [days*(μg/ml)], and 10.5 (ml/day/kg), respectively.

### 3^{rd} Round of Screening

As we found that multiple mutations in heavy chain reduced the potency substantially, we further screened variants, N6LS.C29 through N6LS.C38, incorporating no mutation or single mutations in the heavy chain and up to three mutations in the light chain to identify variants with low affinity to heparin and potency maintained (FIG. 13B). In particular, N6LS.C30 variant incorporating K13E in the heavy chain and R18E in the light chain was selected to assess its PK as it showed most reduced affinity to heparin among variants N6LS.C29 through N6LS.C33 and the potency reduced less than two-fold. From the other group of variants, N6LS.C34 through N6LS.C38, N6LS.C35 incorporating K13E in the heavy chain and a triple mutation, R18E, R42E, and K45E, in the light chain (FIG. 13B), was selected to assess its PK as it showed the most reduced affinity to heparin among all N6LS variants we have tested even though its potency reduced more than two-fold (FIG. 4C). These variants exhibited improved PK parameters, which were statistically significant, compared to their parental: improved half-lives in average of −14 days from 9 days (FIGS. 4D and E, 7, 10B). On a 208-virus panel, N6LS.C1, C15. C30, and C35 neutralized 73%, 78%, 77%, and 73% of viruses at IC80<1 μg/ml, respectively (FIGS. 4F and 4G). In summary, N6LS.C1, C15, C30, and C35 showed substantially improved PK properties with the potency comparable to that of N6LS, of which N6LS.C15 is regarded as the most improved variant when all parameters, such as half-life, the AUC and clearance rate, were considered.

A summary of HIV-1 neutralization, heparin binding, and PK data for several variant N6 antibodies is provided in FIGS. 24A and 24B.

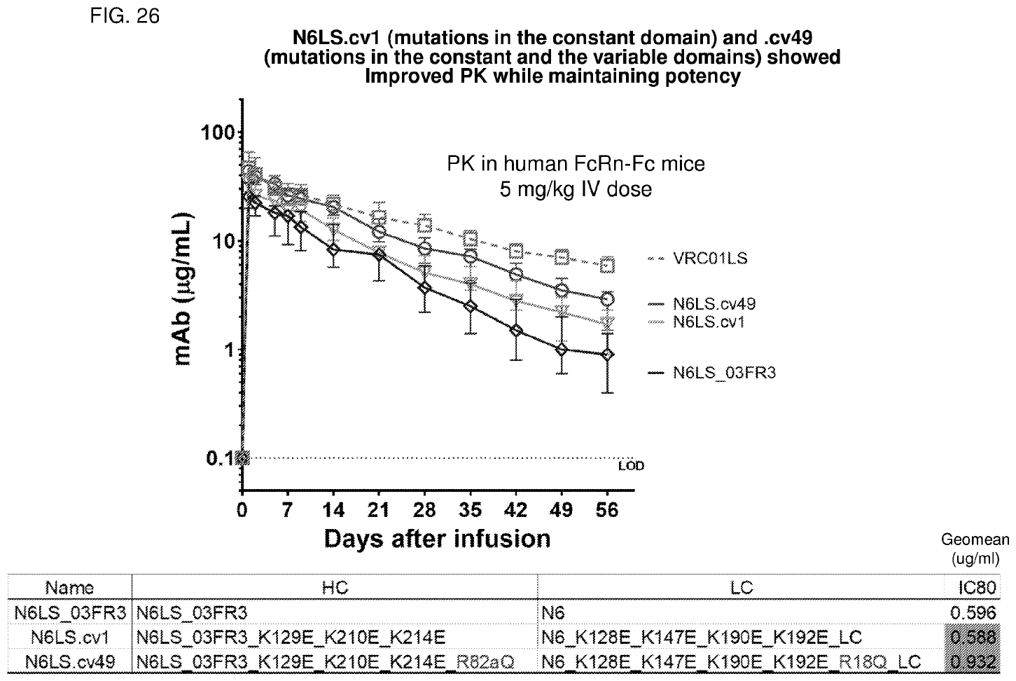

Additional mutations in the VRC07-523LS heavy and light chain constant domains were assessed virus neutralization and pharmacokinetics (FIG. 26).

The N6 antibody was also modified with mutations in the heavy and light chain constant domains and assessed for virus neutralization and pharmacokinetics (FIG. 26). The mutations included K129E, K210E, and K214E in the heavy chain constant domain and K128E, K147E, K190E, and K192E in the light chain constant domain. These were also assessed with the addition of the v49 mutations to the variable regions, R82aQ in the heavy chain variable region and R18Q in the light chain variable region.

Antibody Affinity to Heparin Correlated with Affinity to FcRn at pH 7.4 when the Fab Region is Free to Interact with the Carboxymethyl-Dextran Matrix on the Biosensors

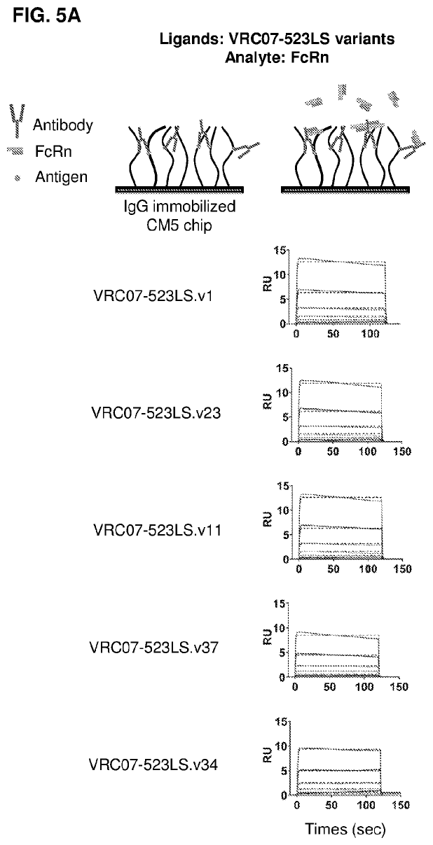

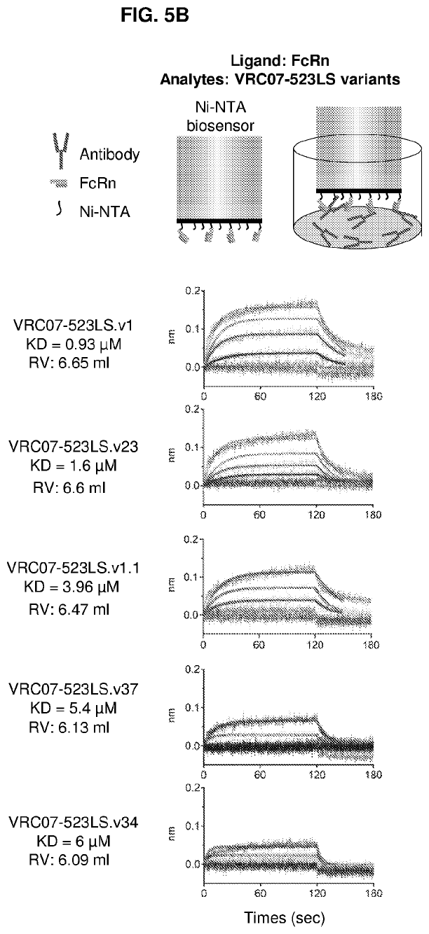

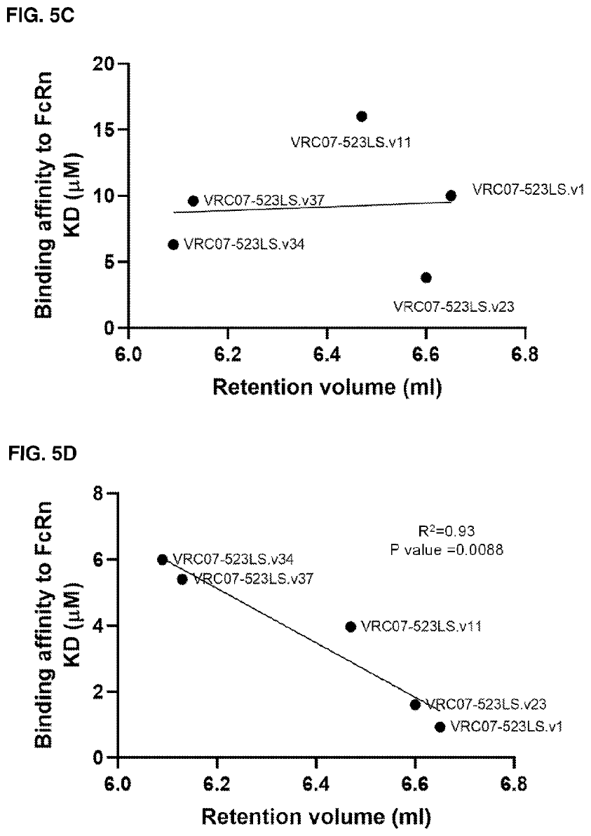

To determine if the reduced net positive charge in the variable domain affects antibody binding affinity to FcRn at pH 7.4, we measured the binding affinity of variants to FcRn using surface plasmon resonance (SPR) and bio-layer interferometry (BLI) in two different formats: i) an experimental setup where a range of concentrations of FcRn was passed over the VRC07-523LS variants captured with gp120 antigen immobilized on a CM5 chip, to measure the antibody Fc-FcRn interaction while avoiding contributions from Fab region (FIG. 5A) and ii) the setup where Ni-NTA biosensors coated with his-tagged FcRn were dipped into the wells containing VRC07-523LS variants, a mimic of antibody Fc and FcRn interaction in physiological condition, where the Fab region is free to interact with the matrix on the biosensor, a surrogate of the membrane components (FIG. 5B). The affinity of the variants to FcRn measured with format i) did not show any significant differences among tested variants. However, the binding affinity measured with format ii) was observed to be in the rank order of VRC07-523LS.v1>VRC07-523LS.v23>VRC07-23LS.v34>VRC07-523LS.v37 from the highest to lowest, which mirrored the rank order of variant's affinity to heparin (FIG. 5B). This demonstrated that the reduced affinity to heparin correlated with reduced affinity to FcRn binding at pH 7.4, suggesting that reduction of net positive charge surface charge also played a role in extension of half-life by facilitating the release of antibodies to the blood stream through the FcRn-mediated antibody recycling.

Arg or Lys Found to be the Most Prevalent at Select Positions in the Variable Domain and are Relatively Conserved, Thereby Substitution of these Residues with Asp or Glu has Potential to Increase the PK of Human Antibodies.

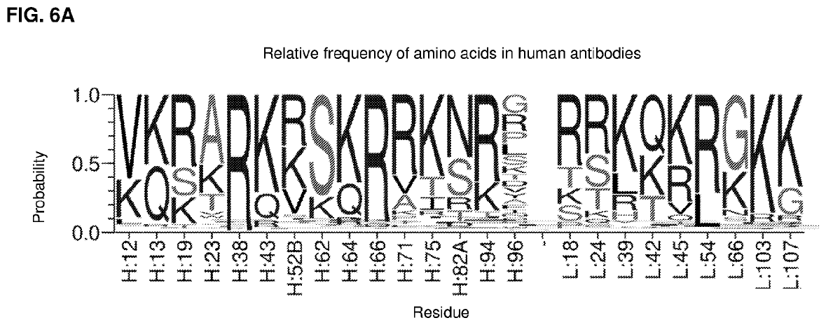

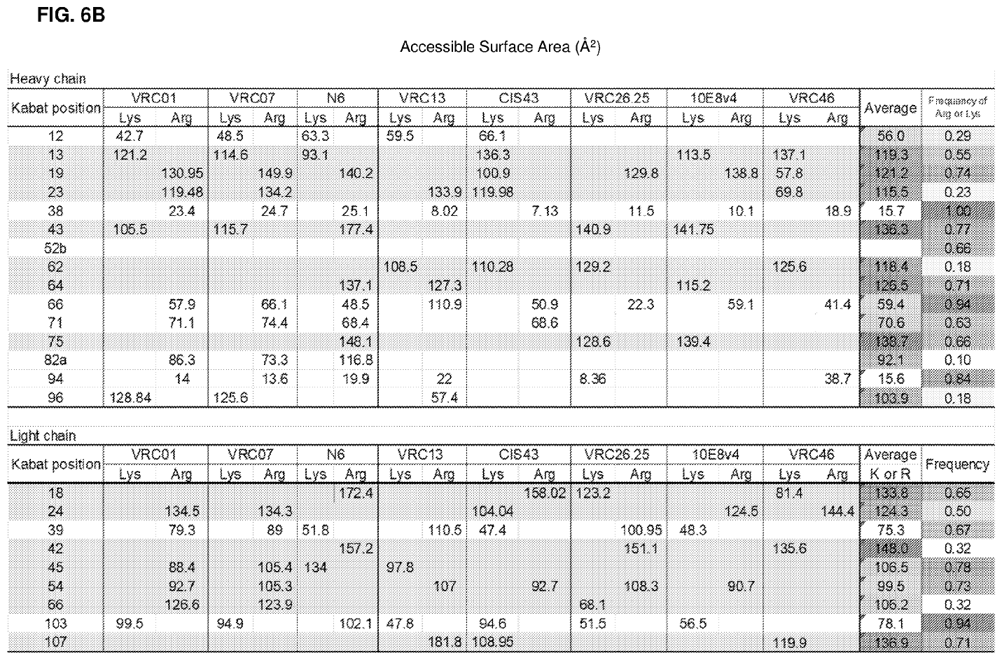

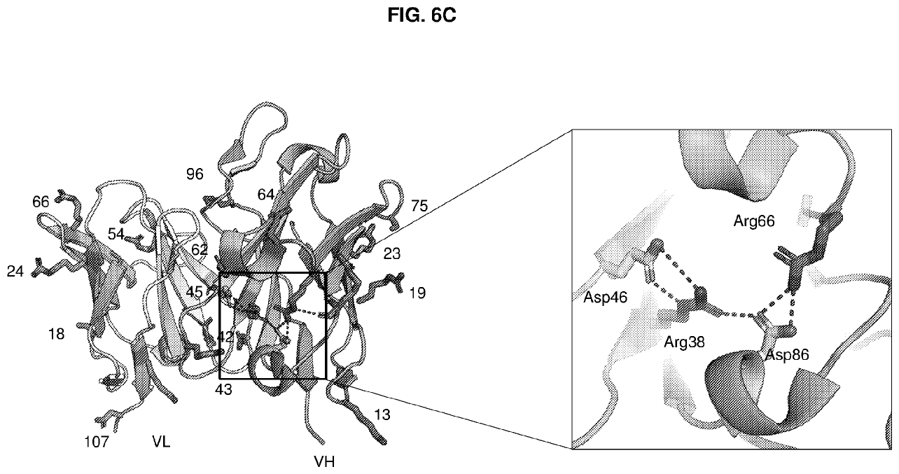

To assess the general applicability of this approach—reducing net positive charge by replacing the select Arg or Lys in the variable domain with Asp, Gln, Glu, or Ser—in improving PK, we utilized abYsis database (abysis.org/abysis/searches/distributions/distributions.cgi) (Swindells et al., 2017), obtained the relative frequency of amino acids in the variable domain of human antibodies, and found Arg and Lys to be most prevalent among other amino acids at select positions: 13, 19, 23, 38, 43, 52B, 62, 64, 71, 75, 82A, 94, and 96 on heavy chain and 18, 24, 39, 42, 45, 54, 66, 103, 107, and 108 on light chain (in Kabat numbering), with the relative frequency of either Arg or Lys from ˜18% to ˜99% (FIG. 6A). The relative frequency of Arg or Lys among HIV-1 antibodies trended with that of human antibodies. Next, to evaluate if these residues are suitable for substitution with negative charged or polar residues without compromising structural integrity, we calculated the accessible surface area (ASA) of these residues from the structures of 8 different HIV-1 antibodies, and found that heavy chain Arg38 and Arg66, which are conserved 99% and 94%, were substantially buried, with ASA of ˜16 A² and ˜59 A², respectively (FIG. 6B). Furthermore, Arg38 interacted with Asp46 and Asp86, and Arg66 interacted with Asp86 via salt bridges (PDB ID 40LW) (FIG. 6C). Interestingly, Asp 46 and Asp86 were also highly conserved (the relative frequencies of 96% and 99%, respectively) in human antibodies, indicating that the four-residue interaction is critical for maintaining the structural integrity of the heavy chain, and therefore any substitutions that could break these interactions would result in loss of function. However, we found either Arg or Lys to be most prevalent and highly exposed at positions 13, 19, 23, 43, 62, 64, and 75 on heavy chain, and 18, 24, 42, 45, 54, 66, and 107 on light chain in human antibodies (FIGS. 6A and B). Notably, VRC07-523LS and N6LS variants incorporating Arg or Lys to Asp, Gln, Glu, or Ser mutations at positions Arg18, Arg24, Arg42, and Lys45 on light chain, and at Lys13, Arg19, Arg23, and Lys96 on heavy chain, which are among highly conserved residues with high ASA, showed improved PK.

### Discussion

Here we determined the basis of the reduced polyreactivity and increased in vivo half-life of VRC01-class antibodies to be four Asp residues introduced by 03FR3 loop insertion and applied this finding to engineer highly potent VRC07-523LS and N6LS antibody variants, with improved PK properties by reducing net positive charge of the variable domain. We utilized heparin affinity chromatography, polyreactivity assessment, and neutralizing potency to screen a panel of antibody variants for improved PK with their potencies comparable to the parental.

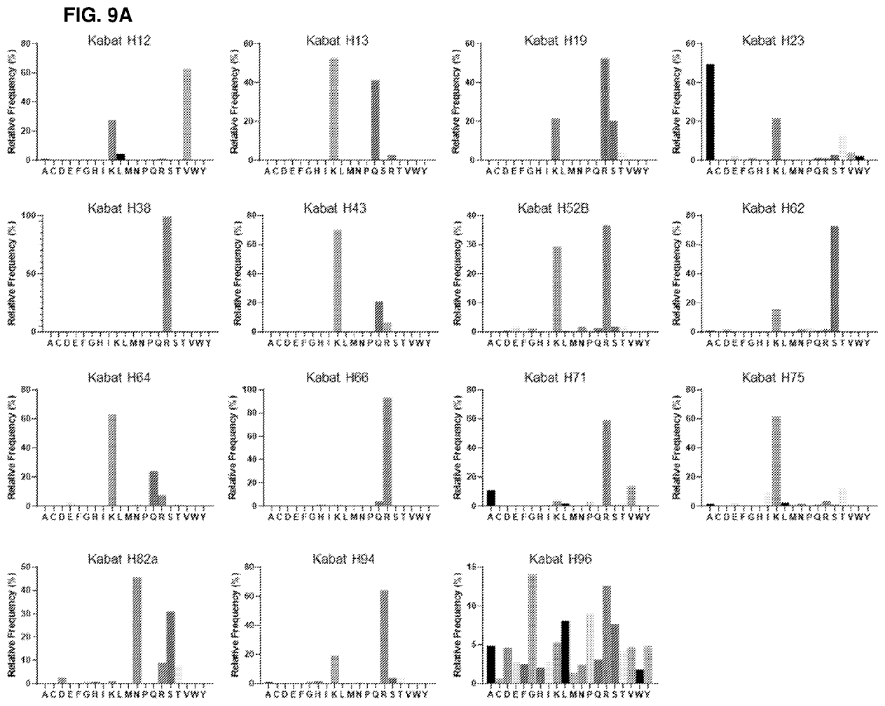

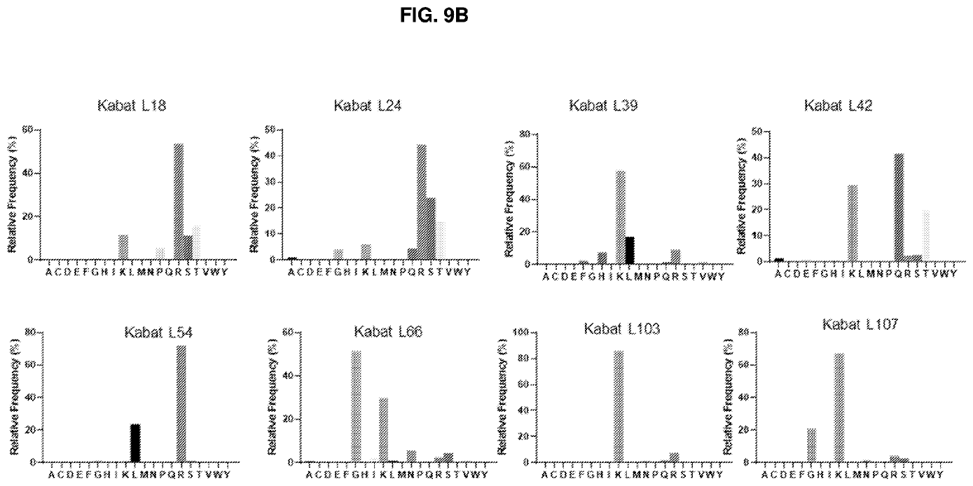

What we have found, which may be generally applicable to improve the PK of other therapeutic antibodies, includes: i) VRC07-523LS and N6LS variants incorporating Arg or Lys to Asp, Gln, Glu, or Ser mutation showed reduced affinity to heparin in the rank order of Glu>Asp>Gln>Ser mutation in general with Glu being most reduced (FIG. 12A), ii) the reduced affinity to heparin, however, resulted in reduced potency, which also mirrored the rank order of reduced affinity to heparin: Glu>Asp>Gln>Ser (FIG. 9D), iii) the same Arg to Asp mutation or Lys to Glu mutation exhibited different degree of affinity to heparin and potency losses depending on where they are located. For example, K43E incorporated in VRC07-523LS.v16 and VRC07-523LS.v20 heavy chain exhibited more reduced affinity to heparin and reduced potency than K13E and K96E (FIG. 12A), iv) VRC07-523LS variants exhibited more dramatic change in affinity to heparin and in potency by mutations introduced in the light chain than the heavy chain, while N6LS was the other way around, suggesting that a potential hotspot for improving PK is antibody-dependent, and v) the VRC07-523LS and N6LS variant affinities to heparin inversely correlated with improvement in PK (FIG. 9C), but with no statistical significance in part due to relatively a small sample size. Nonetheless, VRC07-523LS and N6LS variants with these mutations all showed improved PK properties significantly, compared to their parentals (FIGS. 9A and 9B). It is also generally true that the more net positive charge reducing mutations were more effective in improving PK. However, multiple charge mutations led to more loss in potency. Therefore, to successfully implement this approach, it is necessary to minimize the loss of potency by screening Arg or Lys residues that affect potency minimally when substituted with either Asp or Glu. Overall, Arg or Lys to Glu or Asp mutations resulted in the greatest reduction in affinity to heparin and potency, followed by Gln and by Ser mutation (FIG. 9D). Depending on the magnitude of loss in potency, Asp or Glu substitution can be replaced with uncharged polar amino acids such as Asn, Gln, Thr, or Ser to minimize the potency losses, as we found that Gln or Ser mutation preserved the potency more closely to that of wild type than Asp or Glu mutations (FIG. 9D). For example, triple Ser mutations enabled VRC07-523LS.v32 to reduce its affinity to heparin by the same magnitude as that of VRC07-523LS.v14 but to retain its potency more closely to that of the parental than VRC07-523LS.v14 which contained two Asp mutations (FIG. 9D).

These findings and the fact that Arg or Lys that are most prevalent among other amino acids in the variable domain are highly conserved in human antibodies (FIG. 6), and previous studies on effects of charge versus the plasma PK of antibodies (Igawa et al., 2010; Zhou et al., 2004), suggest that the surface charge engineering, i.e. reducing net positive charge in the variable domain, could serve as a promising way to improve the PK of human antibodies. While it has been reported that measurable changes in tissue distribution and kinetics of an antibody can occur with shifts in isoelectric point of about 1 pI unit of more (Boswell et al., 2010), our data showed that most of variants with improved PK exhibited shifts of isoelectric point less than 0.5 pI unit. For example, a single mutation, R24D, in the light chain of VRC07-523LS.v11, with the shifts in isoelectric point of only 0.18 pI unit, substantially increased PK properties while preserving the potency and breadth (FIG. 3D). Arg18 to Asp mutation, R18D, in the N6 light chain in N6LS.C1, also improved PK of N6LS.C1 when combined with heavy chain R82aD mutation, with the shifts in isoelectric point less than 0.5 pI unit (FIG. 4E). These Arg or Lys are highly conserved with the relative frequency of Arg or Lys at light chain 18 and 24 (in Kabat numbering) of 57% and 51%, respectively, among ˜35,000 antibodies (FIGS. 6A and 8), possibly allowing for Arg to Asp or Glu mutation at these locations where positive charge reduction might generally be applicable to improve PK.

Taken together, we demonstrated that surface charge altering approach coupled with structure-guided target residue selection, heparin affinity chromatography, and neutralization assessment is a promising approach to improve PK of therapeutic antibodies.

### REFERENCES

Bernfield et al. (1999). Functions of cell surface heparan sulfate proteoglycans. Annu Rev Biochem 68, 729-777.
Boswell et al. (2010). Effects of charge on antibody tissue distribution and pharmacokinetics. Bioconjug Chem 21, 2153-2163.
Chames et al. (2009). Therapeutic antibodies: successes, limitations and hopes for the future. Br J Pharmacol 157, 220-233.
Corey et al. (2021). Two Randomized Trials of Neutralizing Antibodies to Prevent HIV-1 Acquisition. N Engl J Med 384, 1003-1014.
Dall'Acqua et al. (2006). Properties of human IgG1s engineered for enhanced binding to the neonatal Fc receptor (FcRn). J Biol Chem 281, 23514-23524.
Diskin et al. (2011). Increasing the potency and breadth of an HIV antibody by using structure-based rational design. Science 334, 1289-1293.
Doria-Rose et al. (2012). HIV-1 neutralization coverage is improved by combining monoclonal antibodies that target independent epitopes. J Virol 86, 3393-3397.
Grevys et al. (2015). Fc Engineering of Human IgG1 for Altered Binding to the Neonatal Fc Receptor Affects Fc Effector Functions. J Immunol 194, 5497-5508.
Gruell and Klein (2018). Antibody-mediated prevention and treatment of HIV-1 infection. Retrovirology 15, 73.
Hinton et al. (2004). Engineered human IgG antibodies with longer serum half-lives in primates. J Biol Chem 279, 6213-6216.
Igawa et al. (2010). Reduced elimination of IgG antibodies by engineering the variable region. Protein Eng Des Sel 23, 385-392.
Julg and Barouch (2019). Neutralizing antibodies for HIV-1 prevention. Curr Opin HIV AIDS 14, 318-324.
Kong et al. (2019). Antibody Lineages with Vaccine-Induced Antigen-Binding Hotspots Develop Broad HIV Neutralization. Cell 178, 567-584 e519.
Kraft et al. (2020). Heparin chromatography as an in vitro predictor for antibody clearance rate through pinocytosis. Mabs 12.
Krissinel and Henrick (2007). Inference of macromolecular assemblies from crystalline state. J Mol Biol 372, 774-797.
Lee et al. (2019). An engineered human Fc domain that behaves like a pH-toggle switch for ultra-long circulation persistence. Nature Communications 10.
Liu et al. (2019). Improvement of antibody functionality by structure-guided paratope engraftment. Nat Commun 10, 721.
Lu et al. (2020). Development of therapeutic antibodies for the treatment of diseases. J Biomed Sci 27, 1.
Mackness et al. (2019). Antibody Fc engineering for enhanced neonatal Fc receptor binding and prolonged circulation half-life. Mabs-Austin 11, 1276-1288.
Pepinsky et al. (2010). Improving the solubility of anti-LINGO-1 monoclonal antibody Li33 by isotype switching and targeted mutagenesis. Protein Sci 19, 954-966.
Piche-Nicholas (2018). Changes in complementarity-determining regions significantly alter IgG binding to the neonatal Fc receptor (FcRn) and pharmacokinetics. Mabs 10, 81-94.
Rice et al. (2000). EMBOSS: the European Molecular Biology Open Software Suite. Trends Genet 16, 276-277.
Rudicell et al. (2014). Enhanced potency of a broadly neutralizing HIV-1 antibody in vitro improves protection against lentiviral infection in vivo. J Virol 88, 12669-12682.
Sarzotti-Kelsoe et al. (2014). Optimization and validation of the TZM-bl assay for standardized assessments of neutralizing antibodies against HIV-1. J Immunol Methods 409, 131-146.
Schlothauer et al. (2013). Analytical FcRn affinity chromatography for functional characterization of monoclonal antibodies. MAbs 5, 576-586.
Schoch et al. (2015). Charge-mediated influence of the antibody variable domain on FcRn-dependent pharmacokinetics. Proc Natl Acad Sci USA 112, 5997-6002.
Sievers et al. (2015). Antibody engineering for increased potency, breadth and half-life. Curr Opin HIV AIDS 10, 151-159.
Sigounas et al. (1994). Half-life of polyreactive antibodies. J Clin Immunol 14, 134-140.
Sun et al. (2020). Antigen physiochemical properties allosterically effect the IgG Fc-region and Fc neonatal receptor affinity. MAbs 12, 1802135.
Suzuki et al. (2010). Importance of neonatal FcR in regulating the serum half-life of therapeutic proteins containing the Fc domain of human IgG1: a comparative study of the affinity of monoclonal antibodies and Fc-fusion proteins to human neonatal FcR. J Immunol 184, 1968-1976.
Swindells et al. (2017). abYsis: Integrated Antibody Sequence and Structure-Management, Analysis, and Prediction. J Mol Biol 429, 356-364.
Wang et al. (2011). Monoclonal antibodies with identical Fc sequences can bind to FcRn differentially with pharmacokinetic consequences. Drug Metab Dispos 39, 1469-1477.
Ward et al. (2015). Targeting FcRn for the modulation of antibody dynamics. Mol Immunol 67, 131-141.
Wu et al. (2007). Development of motavizumab, an ultra-potent antibody for the prevention of respiratory syncytial virus infection in the upper and lower respiratory tract. J Mol Biol 368, 652-665.
Xu et al. (2019). Structure, heterogeneity and developability assessment of therapeutic antibodies. Mabs-Austin 11, 239-264.
Zalevsky et al. (2010). Enhanced antibody half-life improves in vivo activity. Nat Biotechnol 28, 157-159.
Zaman et al. (2019). Current strategies in extending half-lives of therapeutic proteins. J Control Release 301, 176-189.
Zhou et al. (2004). Monte Carlo simulations of antibody adsorption and orientation on charged surfaces. J Chem Phys 121, 1050-1057.

### Example 3

### Broad HIV-Neutralizing 10E8 Variant Antibodies with Enhanced Pharmacokinetics Achieved by Reduction of Net Positive Charge on the Variable Domain

This example describes 10E8 antibody variants with reduced affinity to heparin and improved in vivo half-life, with neutralization potency maintained.

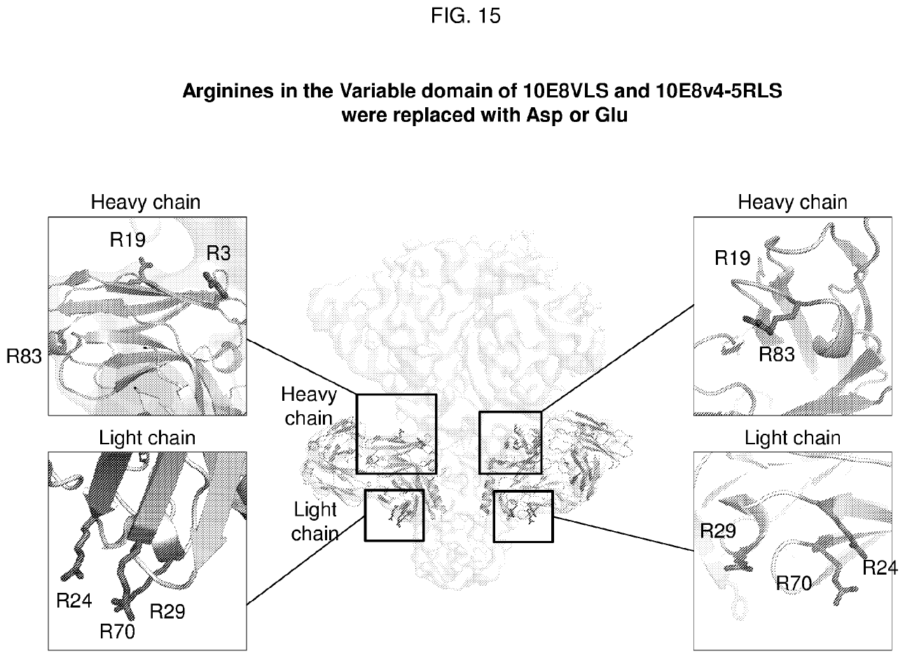

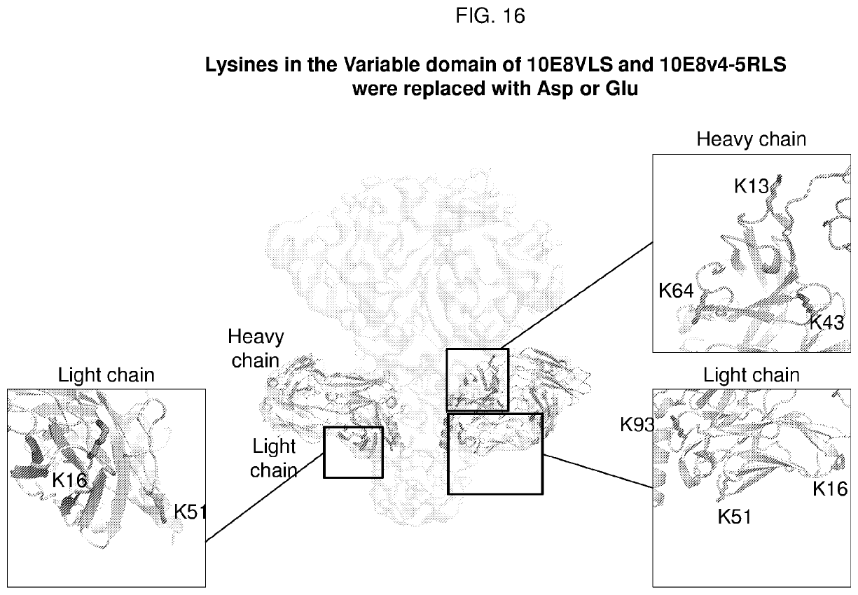

Structure-based design was used to identify exposed arginine and lysine residues for substitution that do not contract epitope for positively charged residues (FIGS. 15 and 16). Multiple residues were selected for mutation, including R3D, R19D, R83D in the heavy chain variable region and R24D, R29D, R70D in the light chain variable region. The mutations or combinations there of were made into 10E8VLS and 10E8v4-5RLS antibodies and assessed for neutralization on a small virus panel.

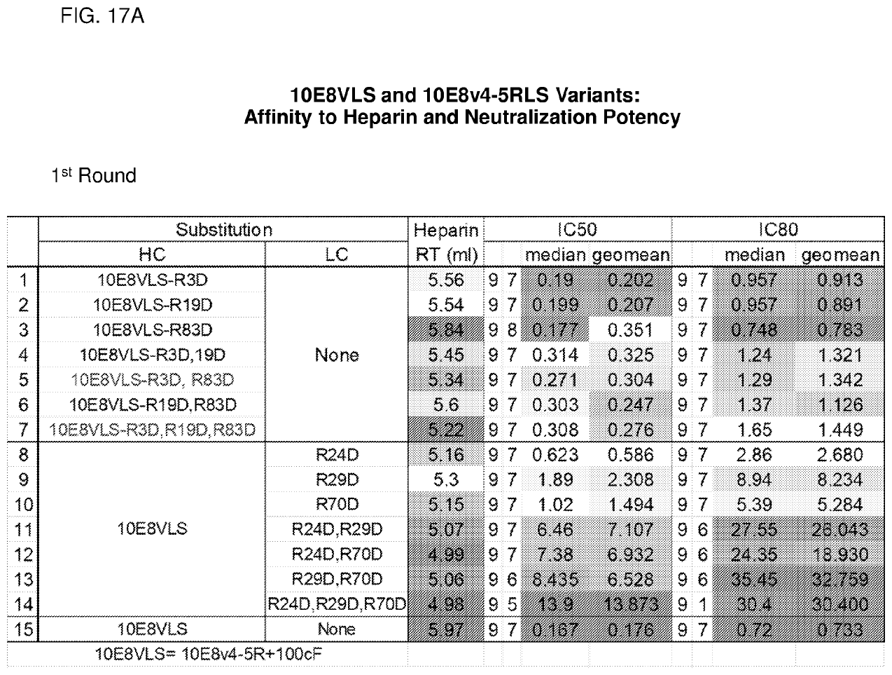

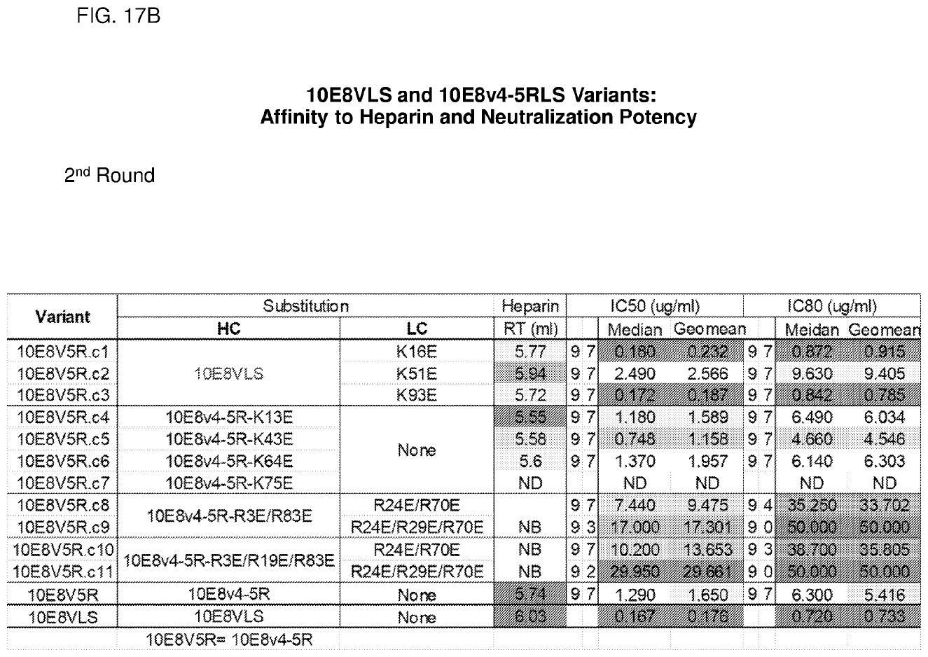

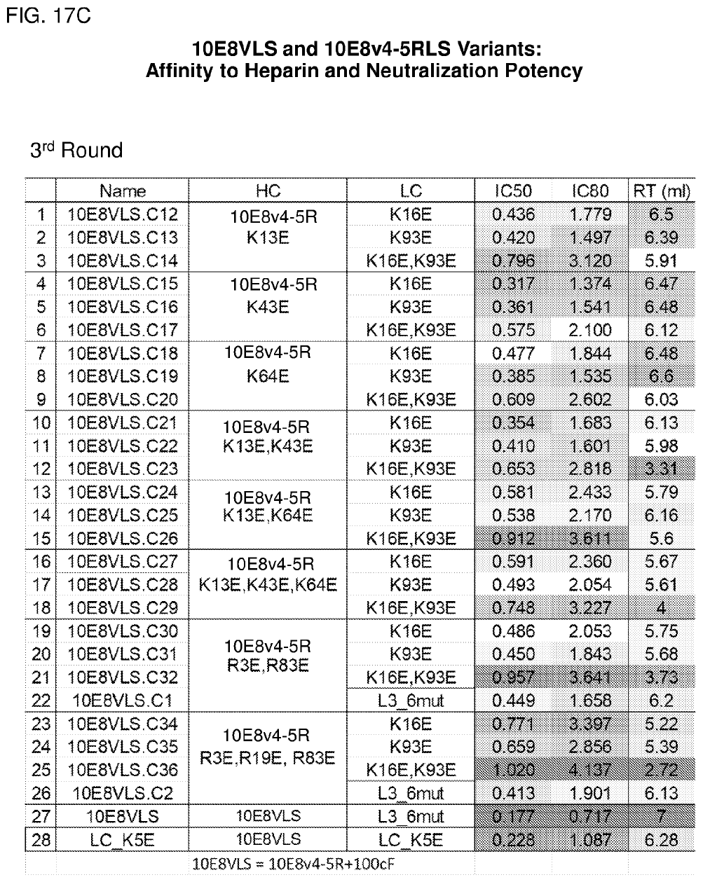

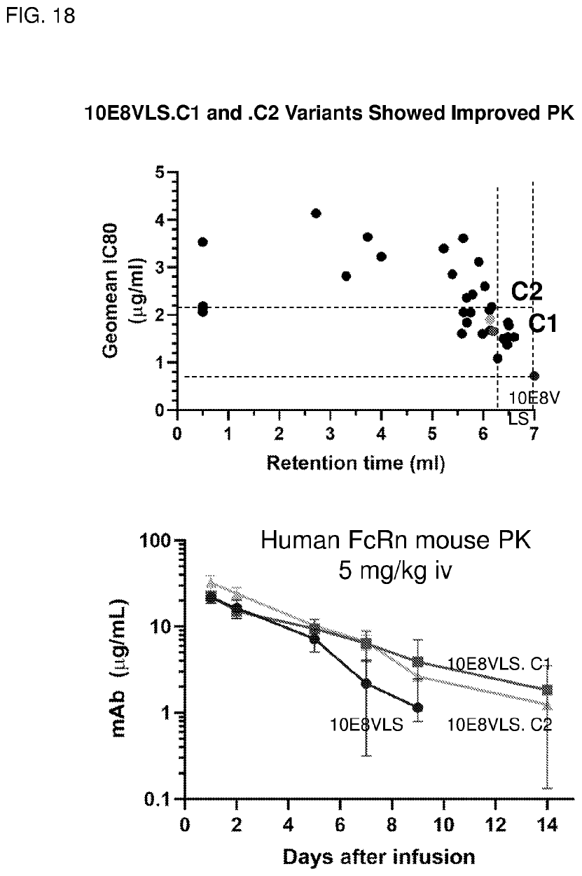

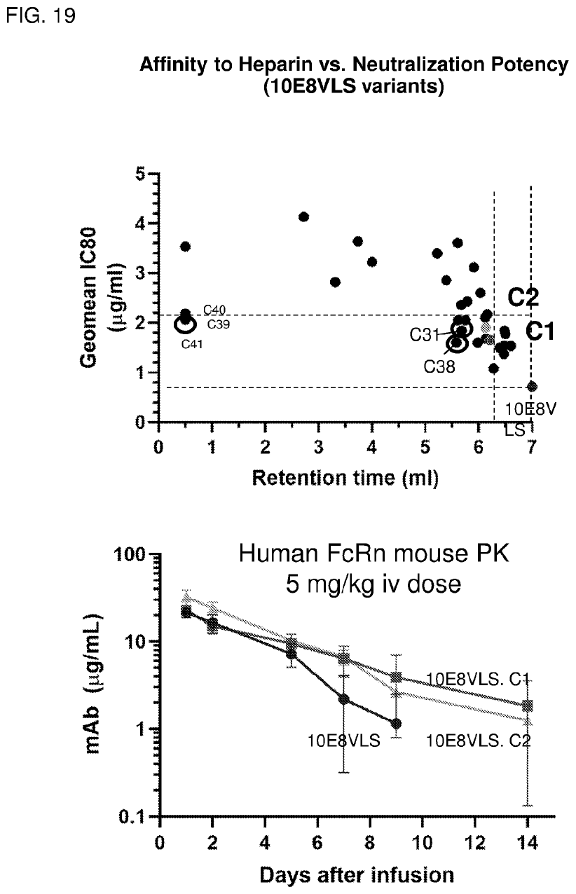

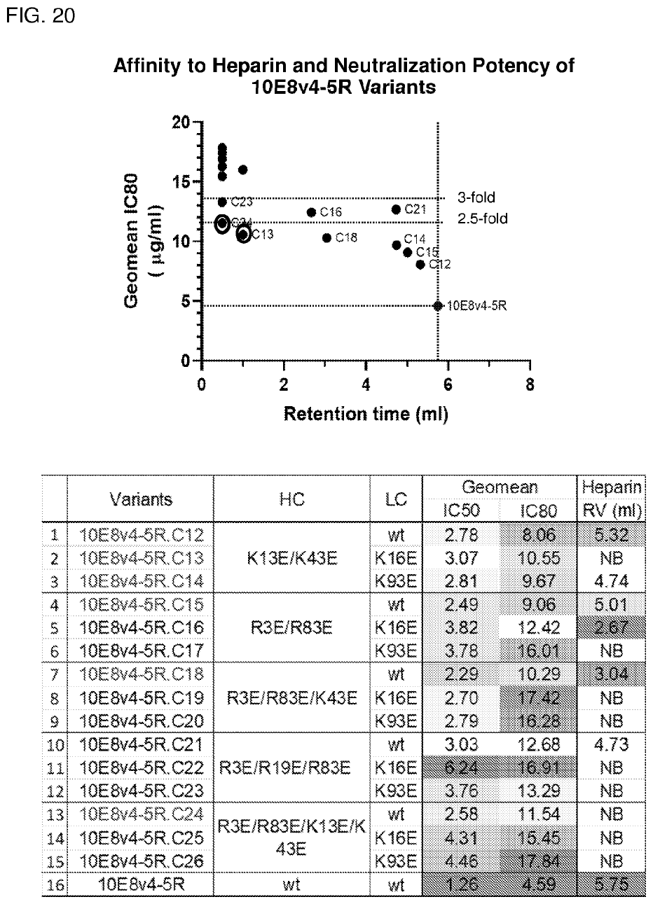

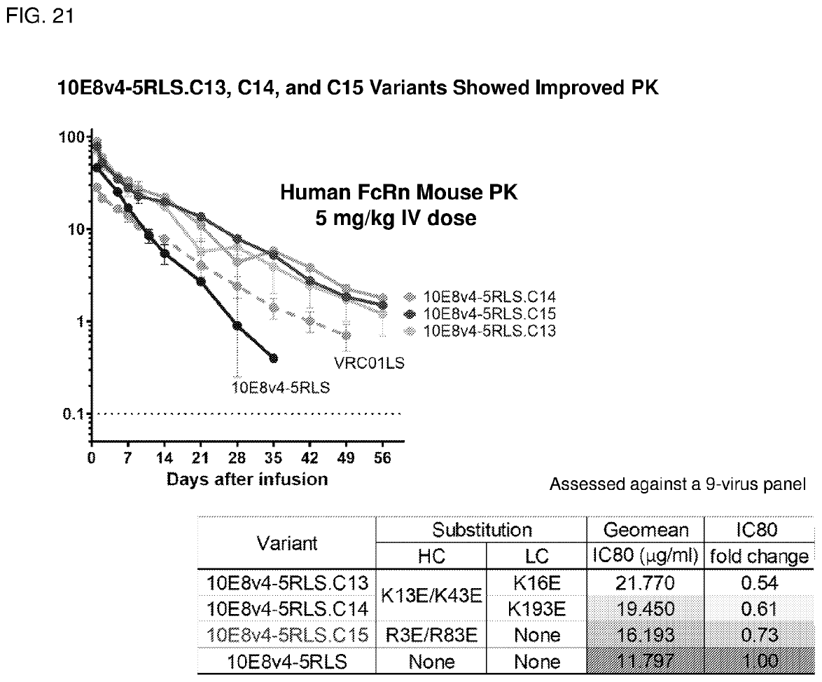

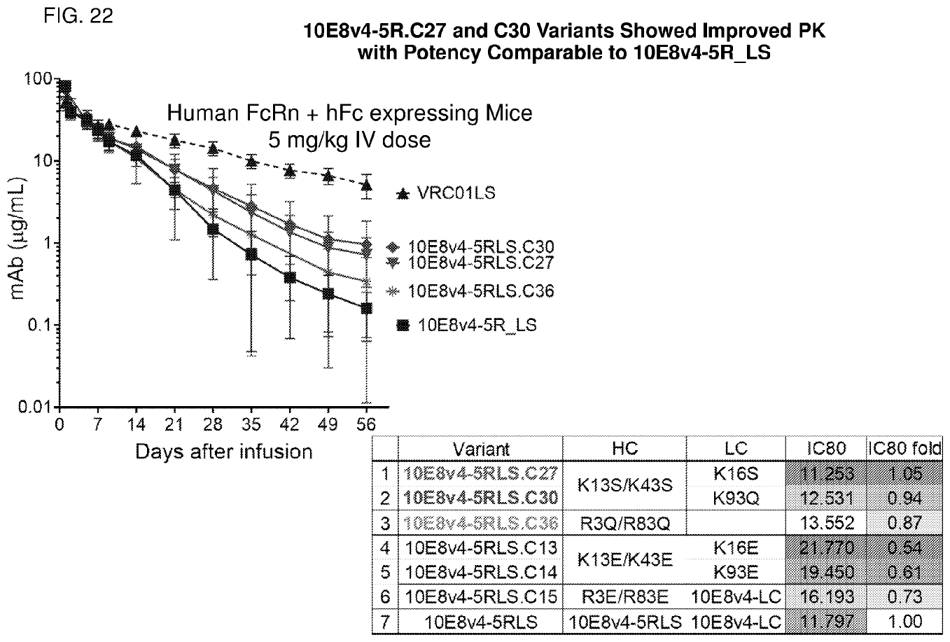

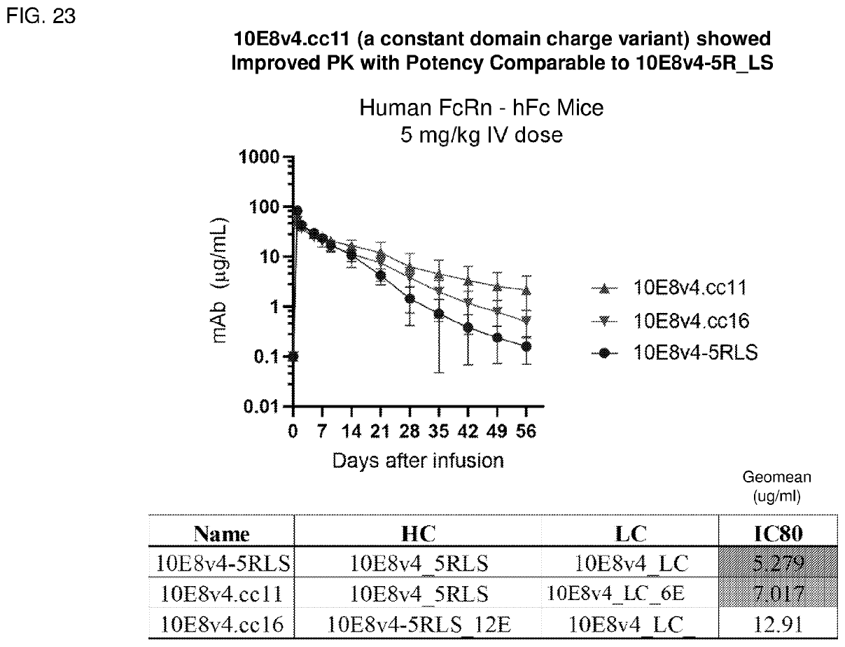

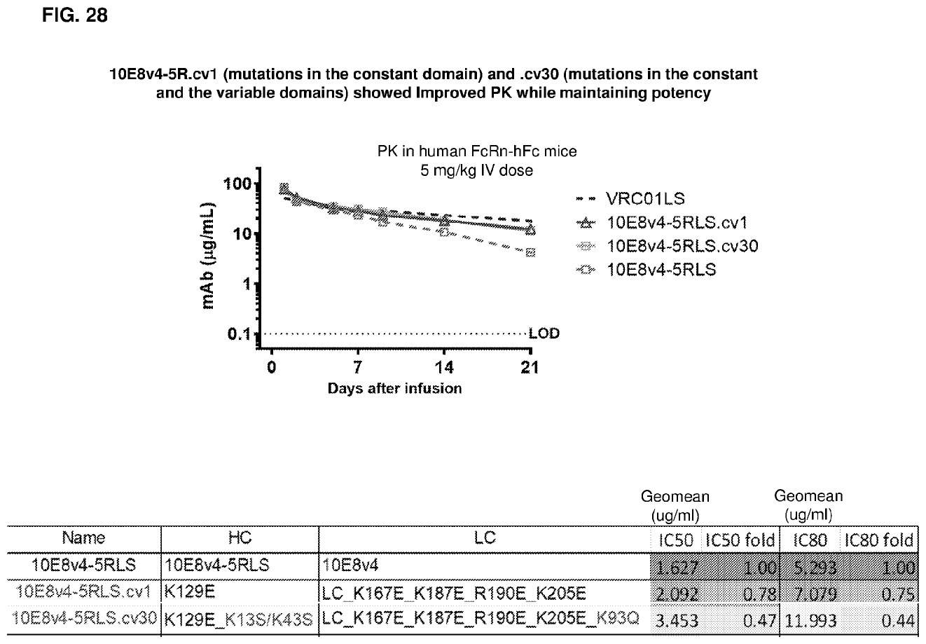

The 10E8v4-5RLS antibody sequences are provided as V_{H} (SEQ ID NO: 41), V_{L} (SEQ ID NO: 42), Heavy Chain (SEQ ID NO: 43), and Light Chain (SEQ ID NO: 44). 10E8VLS is 10E8v4-5RLS further modified with a serine to phenylalanine substitution at Kabat 100c of the heavy chain variable region. Based on a first round of analysis (FIG. 17A), additional mutations were selected for a second round (FIG. 17B), and finally a third round (FIG. 17C). Select variant 10E8 antibodies were further assessed for heparin binding and virus neutralization (FIGS. 18-20, 28), and pharmacokinetics using the human FcRn mouse (FIGS. 21-23, 28).

A summary of HIV-1 neutralization, heparin binding, and PK data for several variant 10E8 antibodies is provided in FIGS. 24A and 24B.

Several identified variants, including 10E8v4-5RLS.C12, 10E8v4-5RLS.C14, 10E8v4-5RLS.C15, 10E8v4-5RLS.C27, 10E8v4-5RLS.C30, 10E8v4.cc11, 10E8v4.ccl6, 10E8v4-5RLS.cvl, and 10E8v4-5RLS.Ccv30 showed a surprising combination of neutralization breadth and potency, lack of autoreactivity, and improved half-life compared to the parent 10E8v4-5RLS antibody.

### Example 4

### ATSα Modified Antibodies

This example describes modified VRC01.23LS, VRC07-523LS, N6-LS, and 10E8v4-5RLS antibodies further modified with addition of the acidic tail of alpha-synuclein (ATS(a) to heparin to improve in vivo half-life while maintaining neutralization potency.

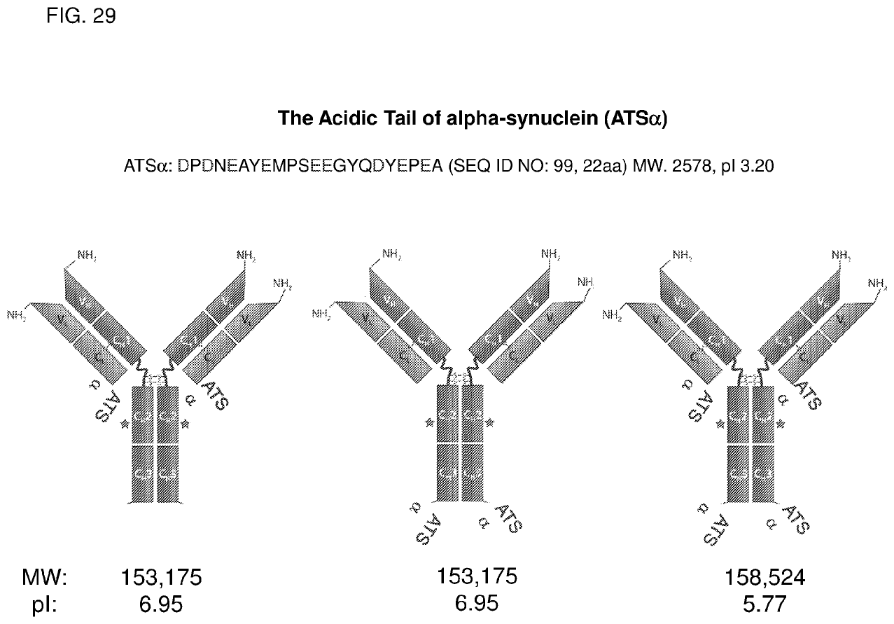

FIG. 29 shows a schematic diagram illustrating modification of antibody IgG with the acidic tail of alpha-synuclein (ATSα, DPDNEAYEMPSEEGYQDYEPEA (SEQ ID NO: 99). The ATSα domain can be place on the C-terminus of the heavy or the light chain, or both.

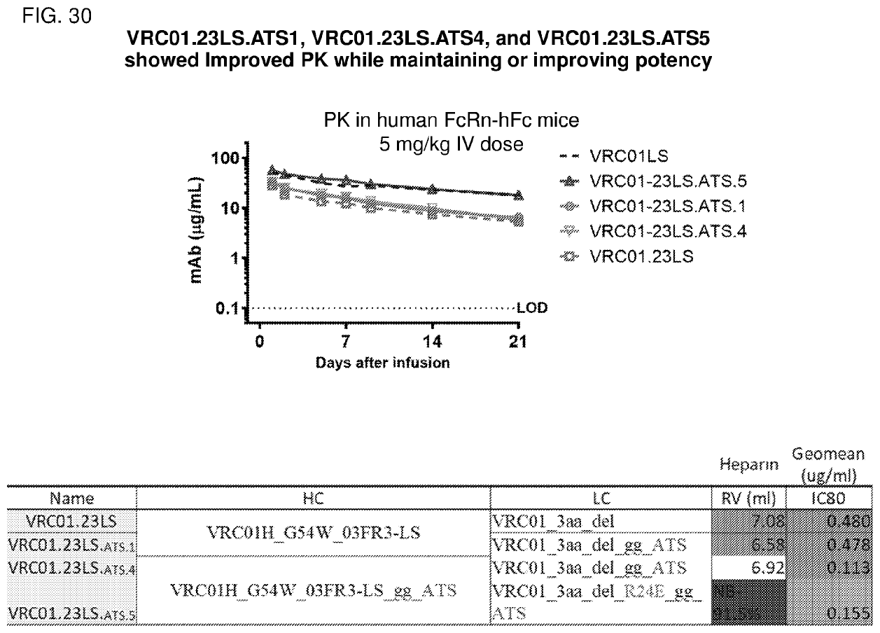

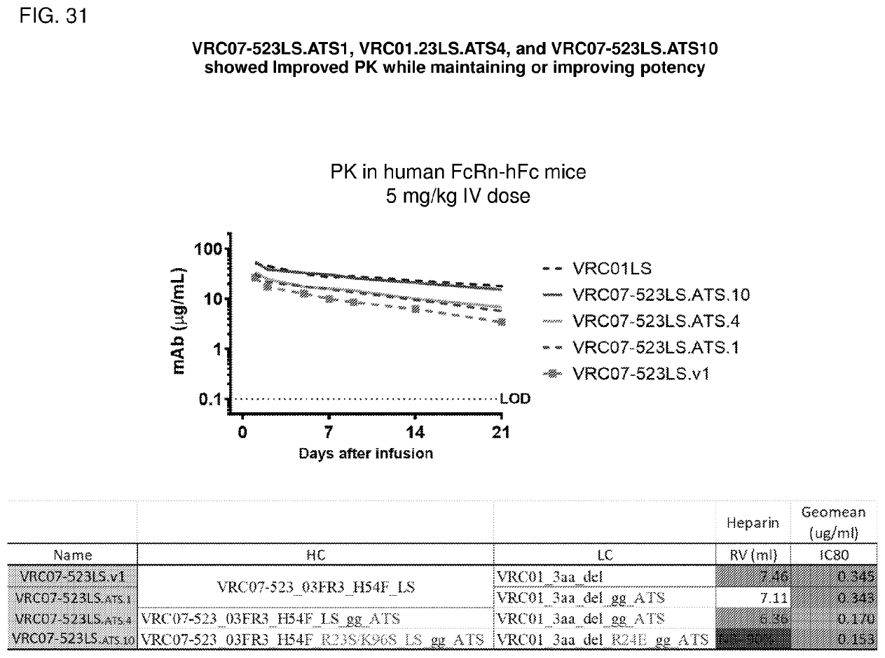

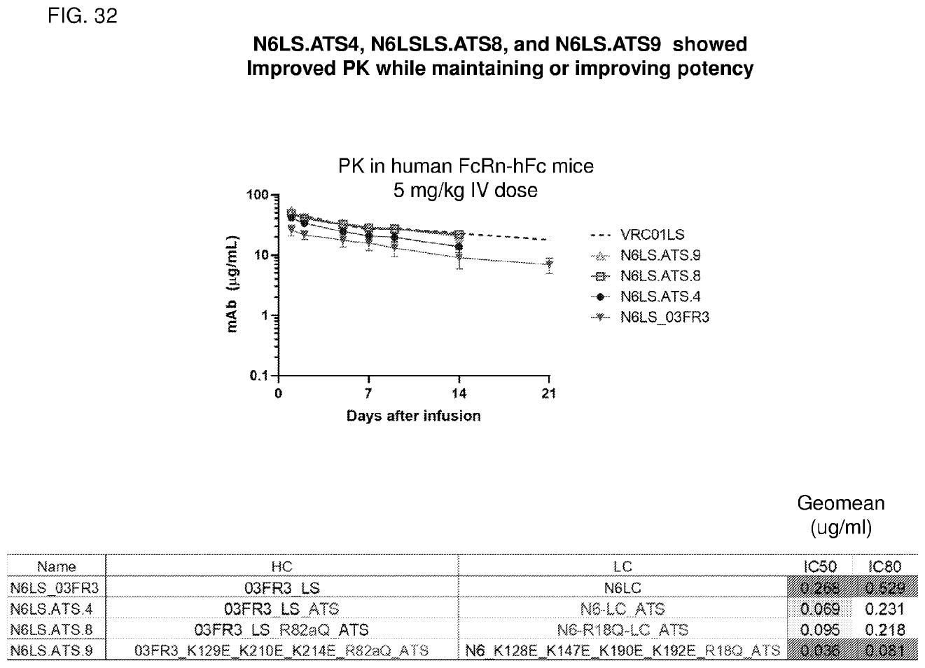

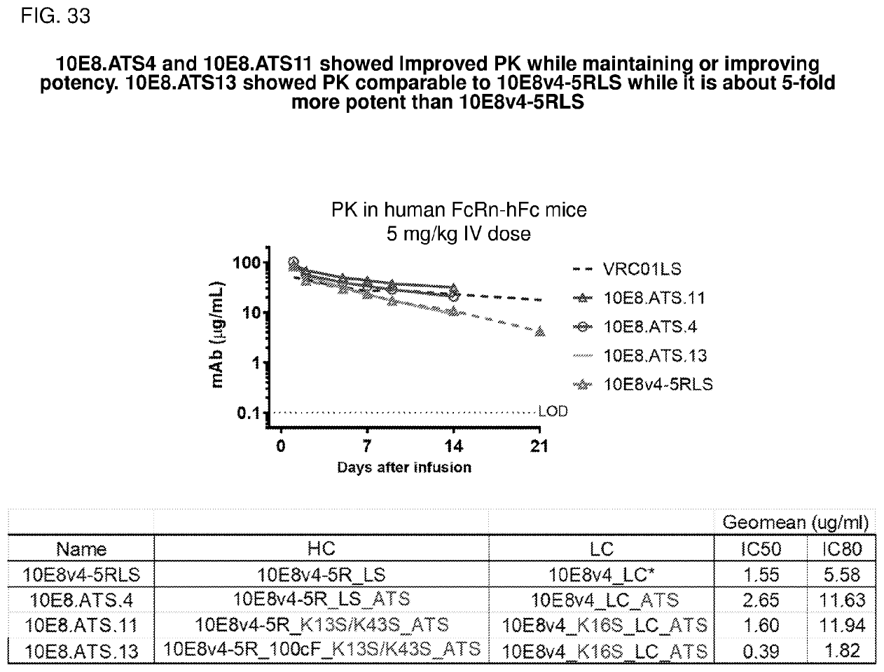

VRC01.23LS, VRC07-523LS, N6-LS, and 10E8v4-5RLS antibodies were modified with the ATSα domain and assessed for heparin binding, antibody neutralization on the 30-virus panel, and pharmacokinetics in the human FcRn mouse (FIGS. 30-33). As shown, the ATSα-modified antibodies exhibited improved PK while maintaining or improving potency.

### Example 5

### Treatment of HIV-1 Using an HIV-1 Env Specific Antibody

This example describes a particular method that can be used to treat HIV-1 infection in a human subject by administration of a disclosed HIV-1 Env-specific antibody. Although particular methods, dosages, and modes of administrations are provided, one skilled in the art will appreciate that variations can be made without substantially affecting the treatment.

Based upon the teaching disclosed herein, HIV-1 infection can be treated by administering a therapeutically effective amount of one or more of the neutralizing mAbs described herein, thereby reducing or eliminating HIV-1 infection.

### Screening Subjects

In particular examples, the subject is first screened to determine if they have an HIV-1 infection. Examples of methods that can be used to screen for HIV-1 infection include a combination of measuring a subject's CD4+ T cell count and the level of HIV-1 virus in serum blood levels. Additional methods using an HIV-1 Env-specific antibody described herein can also be used to screen for HIV-1 infection.

In some examples, HIV-1 testing consists of initial screening with an enzyme-linked immunosorbent assay (ELISA) to detect antibodies to HIV-1. Specimens with a nonreactive result from the initial ELISA are considered HIV-1-negative unless new exposure to an infected partner or partner of unknown HIV-1 status has occurred. Specimens with a reactive ELISA result are retested in duplicate. If the result of either duplicate test is reactive, the specimen is reported as repeatedly reactive and undergoes confirmatory testing with a more specific supplemental test (e.g., Western blot or an immunofluorescence assay (IFA)). Specimens that are repeatedly reactive by ELISA and positive by IFA or reactive by Western blot are considered HIV-positive and indicative of HIV-1 infection. Specimens that are repeatedly ELISA-reactive occasionally provide an indeterminate Western blot result, which may be either an incomplete antibody response to HIV-1 in an infected person, or nonspecific reactions in an uninfected person. IFA can be used to confirm infection in these ambiguous cases. In some instances, a second specimen will be collected more than a month later and retested for subjects with indeterminate Western blot results. In additional examples, nucleic acid testing (e.g., viral RNA or proviral DNA amplification method) can also help diagnosis in certain situations.

The detection of HIV-1 in a subject's blood is indicative that the subject is infected with HIV-1 and is a candidate for receiving the therapeutic compositions disclosed herein. Moreover, detection of a CD4+ T cell count below 350 per microliter, such as 200 cells per microliter, is also indicative that the subject is likely to have an HIV-1 infection.

Pre-screening is not required prior to administration of the therapeutic compositions disclosed herein

### Pre-Treatment of Subjects

In particular examples, the subject is treated prior to administration of a therapeutic agent that includes one or more antiretroviral therapies known to those of skill in the art. However, such pre-treatment is not always required, and can be determined by a skilled clinician.

### Administration of Therapeutic Compositions

Following subject selection, a therapeutically effective dose of a HIV-1 Env-specific antibody described herein is administered to the subject (such as an adult human or a newborn infant either at risk for contracting HIV-1 or known to be infected with HIV-1). Additional agents, such as anti-viral agents, can also be administered to the subject simultaneously or prior to or following administration of the disclosed agents. Administration can be achieved by any method known in the art, such as oral administration, inhalation, intravenous, intramuscular, intraperitoneal, or subcutaneous.

The amount of the composition administered to prevent, reduce, inhibit, and/or treat HIV-1 or a condition associated with it depends on the subject being treated, the severity of the disorder, and the manner of administration of the therapeutic composition. Ideally, a therapeutically effective amount of an agent is the amount sufficient to prevent, reduce, and/or inhibit, and/or treat the condition (e.g., HIV-1) in a subject without causing a substantial cytotoxic effect in the subject. An effective amount can be readily determined by one skilled in the art, for example using routine trials establishing dose response curves. As such, these compositions may be formulated with an inert diluent or with a pharmaceutically acceptable carrier.

In one specific example, antibodies are administered at 5 mg per kg every two weeks or 10 mg per kg every two weeks. In another example, antibodies or antibody fragments are administered at 50 μg per kg given twice a week for 2 to 3 weeks.

Administration of the therapeutic compositions can be taken long term (for example over a period of months or years).

### Assessment

Following the administration of one or more therapies, subjects with HIV-1 can be monitored for reductions in HIV-1 levels, increases in a subject's CD4+ T cell count, or reductions in one or more clinical symptoms associated with HIV-1 disease. In particular examples, subjects are analyzed one or more times, starting 7 days following treatment. Subjects can be monitored using any method known in the art. For example, biological samples from the subject, including blood, can be obtained and alterations in HIV-1 or CD4+ T cell levels evaluated.

### Additional Treatments

In particular examples, if subjects are stable or have a minor, mixed or partial response to treatment, they can be re-treated after re-evaluation with the same schedule and preparation of agents that they previously received for the desired amount of time, including the duration of a subject's lifetime. A partial response is a reduction, such as at least a 10%, at least 20%, at least 30%, at least 40%, at least 50%, or at least 70% in HIV-1 infection, HIV-1 replication or combination thereof. A partial response may also be an increase in CD4+ T cell count such as at least 350 T cells per microliter.

### Example 6

### Antibody Sequences

The following table and list of sequences provides a summary of VH, VL, heavy chain, and light chain SEQ ID NOs for antibodies provided herein:

| | | | | |
|---|---|---|---|---|
| | | | Heavy | Light |
| | VH | VL | Chain | Chain |
| | SEQ | SEQ | SEQ | SEQ |
| Name | ID NO | ID NO | ID NO | ID NO |

Variant VRC07 antibodies

| | | | | |
|---|---|---|---|---|
| VRC07-523LS.v1 | 1 | 2 | 3 | 4 |
| VRC07-523LS.v11 | 1 | 5 | 3 | 6 |
| VRC07-523LS.v14 | 7 | 5 | 8 | 6 |
| VRC07-523LS.v21 | 9 | 10 | 11 | 12 |
| VRC07-523LS.v26 | 13 | 14 | 15 | 16 |
| VRC07-523LS.v32 | 17 | 14 | 18 | 16 |
| VRC07-523LS.v34 | 17 | 10 | 18 | 12 |
| VRC07-523LS.cv1 | 1 | 2 | 57 | 58 |
| VRC07-523LS.cv34 | 59 | 10 | 60 | 61 |
| VRC07-523LS.ATS1 | 1 | 2 | 3 | 79 |
| VRC07-523LS.ATS4 | 1 | 2 | 80 | 81 |
| VRC07-523LS.ATS10 | 17 | 10 | 82 | 83 |

Variant N6 antibodies

| | | | | |
|---|---|---|---|---|
| N6LS | 19 | 20 | 21 | 22 |
| N6LS.C1 | 23 | 24 | 25 | 26 |
| N6LS.C15 | 19 | 27 | 21 | 28 |
| N6LS.C30 | 29 | 30 | 31 | 32 |
| N6LS.C35 | 29 | 27 | 31 | 28 |
| N6LS.C47 | 19 | 33 | 21 | 34 |
| N6LS.C49 | 35 | 33 | 36 | 34 |
| N6LS.C51 | 37 | 33 | 38 | 34 |
| N6LS.C58 | 39 | 33 | 40 | 34 |
| N6LS.cv1 | 19 | 20 | 62 | 63 |
| N6LS.cv49 | 35 | 33 | 64 | 97 |
| N6LS.ATS4 | 19 | 20 | 84 | 85 |
| N6LS.ATS8 | 23 | 33 | 86 | 87 |
| N6LS.ATS9 | 35 | 33 | 88 | 89 |

Variant 10E8 Antibodies

| | | | | |
|---|---|---|---|---|
| 10E8v4-5RLS | 41 | 42 | 43 | 44 |
| 10E8v4-5RLS.C12 | 45 | 42 | 46 | 44 |
| 10E8v4-5RLS.C13 | 45 | 47 | 46 | 48 |
| 10E8v4-5RLS.C14 | 45 | 49 | 46 | 50 |
| 10E8v4-5RLS.C15 | 51 | 42 | 52 | 44 |
| 10E8v4-5RLS.C18 | 53 | 42 | 53 | 44 |
| 10E8v4-5RLS.C24 | 55 | 42 | 56 | 44 |
| 10E8v4-5RLS.C27 | 65 | 66 | 67 | 68 |
| 10E8v4-5RLS.C30 | 65 | 69 | 67 | 70 |
| 10E8v4.cc11 | 41 | 42 | 43 | 71 |
| 10E8v4.cc16 | 41 | 42 | 72 | 44 |
| 10E8v4-5RLS.cv1 | 41 | 42 | 73 | 74 |
| 10E8v4-5RLS.cv30 | 65 | 69 | 75 | 76 |
| 10E8.ATS4 | 41 | 42 | 90 | 91 |
| 10E8.ATS11 | 65 | 66 | 92 | 93 |
| 10E8.ATS13 | 98 | 66 | 94 | 93 |

Variant VRC01 Antibodies

| | | | | |
|---|---|---|---|---|
| VRC01.23LS.cv1 | 77 | 2 | 78 | 58 |
| VRC01.23LS.cv34 | 59 | 10 | 60 | 61 |
| VRC01.23LS.ATS1 | 77 | 2 | 95 | 79 |
| VRC01.23LS.ATS4 | 77 | 2 | 96 | 79 |
| VRC01.23LS.ATS5 | 77 | 10 | 96 | 83 |

VRC07-523LS.v1
V_{H}
(SEQ ID NO: 1)
QVRLSQSGGQMKKPGDSMRISCRASGYEFINCPINWIRLAPGKRPEWMGWMKPRFGAVSYARQLQGRVTMTRQLSQD
PDDPDWGTAFLELRSLTSDDTAVYFCTRGKYCTARDYYNWDFEHWGQGTPVTVSS
V_{L}
(SEQ ID NO: 2)
LTQSPGTLSLSPGETAIISCRTSQYGSLAWYQQRPGQAPRLVIYSGSTRAAGIPDRFSGSRWGPDYNLTISNLESGD
FGVYYCQQYEFFGQGTKVQVDIK
Heavy Chain
(SEQ ID NO: 3)
QVRLSQSGGQMKKPGDSMRISCRASGYEFINCPINWIRLAPGKRPEWMGWMKPRFGAVSYARQLQGRVTMTRQLSQD
PDDPDWGTAFLELRSLTSDDTAVYFCTRGKYCTARDYYNWDFEHWGQGTPVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 4)
LTQSPGTLSLSPGETAIISCRTSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
VRC07-523LS.v11
V_{H}
(SEQ ID NO: 1)
QVRLSQSGGQMKKPGDSMRISCRASGYEFINCPINWIRLAPGKRPEWMGWMKPRFGAVSYARQLQGRVTMTRQLSQD
PDDPDWGTAFLELRSLTSDDTAVYFCTRGKYCTARDYYNWDFEHWGQGTPVTVSS
V_{L}
(SEQ ID NO: 5)
LTQSPGTLSLSPGETAIISCDTSQYGSLAWYQQRPGQAPRLVIYSGSTRAAGIPDRFSGSRWGPDYNLTISNLESGD
FGVYYCQQYEFFGQGTKVQVDIK
Heavy Chain
(SEQ ID NO: 3)
QVRLSQSGGQMKKPGDSMRISCRASGYEFINCPINWIRLAPGKRPEWMGWMKPRFGAVSYARQLQGRVTMTRQLSQD
PDDPDWGTAFLELRSLTSDDTAVYFCTRGKYCTARDYYNWDFEHWGQGTPVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 6)
LTQSPGTLSLSPGETAIISCDTSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
VRC07-523LS.v14
V_{H}
(SEQ ID NO: 7)
QVRLSQSGGQMKKPGDSMRISCDASGYEFINCPINWIRLAPGKRPEWMGWMKPRFGAVSYARQLQGRVTMTRQLSQD
PDDPDWGTAFLELRSLTSDDTAVYFCTRGKYCTARDYYNWDFEHWGQGTPVTVSS
V_{L}
(SEQ ID NO: 5)
LTQSPGTLSLSPGETAIISCDTSQYGSLAWYQQRPGQAPRLVIYSGSTRAAGIPDRESGSRWGPDYNLTISNLESGD
FGVYYCQQYEFFGQGTKVQVDIK
Heavy Chain
(SEQ ID NO: 8)
QVRLSQSGGQMKKPGDSMRISCDASGYEFINCPINWIRLAPGKRPEWMGWMKPRFGAVSYARQLQGRVTMTRQLSQD
PDDPDWGTAFLELRSLTSDDTAVYFCTRGKYCTARDYYNWDFEHWGQGTPVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVLHEALHSHYTQKSLSLSPGK
Light chain
(SEQ ID NO: 6)
LTQSPGTLSLSPGETAIISCDTSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
VRC07-523LS.v21
V_{H}
(SEQ ID NO: 9)
QVRLSQSGGQMKKPGDSMRISCDASGYEFINCPINWIRLAPGKRPEWMGWMKPRFGAVSYARQLQGRVTMTRQLSQD
PDDPDWGTAFLELRSLTSDDTAVYFCTRGEYCTARDYYNWDFEHWGQGTPVTVSS
V_{L}
(SEQ ID NO: 10)
LTQSPGTLSLSPGETAIISCETSQYGSLAWYQQRPGQAPRLVIYSGSTRAAGIPDRFSGSRWGPDYNLTISNLESGD
FGVYYCQQYEFFGQGTKVQVDIK
Heavy Chain
(SEQ ID NO: 11)
QVRLSQSGGQMKKPGDSMRISCDASGYEFINCPINWIRLAPGKRPEWMGWMKPRFGAVSYARQLQGRVTMTRQLSQD
PDDPDWGTAFLELRSLTSDDTAVYFCTRGEYCTARDYYNWDFEHWGQGTPVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 12
LTQSPGTLSLSPGETAIISCETSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
VRC07-523LS.v26
V_{H}
(SEQ ID NO: 13)
QVRLSQSGGQMKKPGDSMRISCSASGYEFINCPINWIRLAPGKRPEWMGWMKPRFGAVSYARQLQGRVTMTRQLSQD
PDDPDWGTAFLELRSLTSDDTAVYFCTRGKYCTARDYYNWDFEHWGQGTPVTVSS
V_{L}
(SEQ ID NO: 14)
LTQSPGTLSLSPGETAIISCSTSQYGSLAWYQQRPGQAPRLVIYSGSTRAAGIPDRFSGSRWGPDYNLTISNLESGD
FGVYYCQQYEFFGQGTKVQVDIK
Heavy Chain
(SEQ ID NO: 15)
QVRLSQSGGQMKKPGDSMRISCSASGYEFINCPINWIRLAPGKRPEWMGWMKPRFGAVSYARQLQGRVTMTRQLSQD
PDDPDWGTAFLELRSLTSDDTAVYFCTRGKYCTARDYYNWDFEHWGQGTPVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 16)
LTQSPGTLSLSPGETAIISCSTSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
VRC07-523LS.v32
V_{H}
(SEQ ID NO: 17)
QVRLSQSGGQMKKPGDSMRISCSASGYEFINCPINWIRLAPGKRPEWMGWMKPRFGAVSYARQLQGRVTMTRQLSQD
PDDPDWGTAFLELRSLTSDDTAVYFCTRGSYCTARDYYNWDFEHWGQGTPVTVSS
V_{L}
(SEQ ID NO: 14)
LTQSPGTLSLSPGETAIISCSTSQYGSLAWYQQRPGQAPRLVIYSGSTRAAGIPDRFSGSRWGPDYNLTISNLESGD
FGVYYCQQYEFFGQGTKVQVDIK
Heavy Chain
(SEQ ID NO: 18)
QVRLSQSGGQMKKPGDSMRISCSASGYEFINCPINWIRLAPGKRPEWMGWMKPRFGAVSYARQLQGRVTMTRQLSQD
PDDPDWGTAFLELRSLTSDDTAVYFCTRGSYCTARDYYNWDFEHWGQGTPVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 16)
LTQSPGTLSLSPGETAIISCSTSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
VRC07-523LS.v34
V_{H}
(SEQ ID NO: 17)
QVRLSQSGGQMKKPGDSMRISCSASGYEFINCPINWIRLAPGKRPEWMGWMKPRFGAVSYARQLQGRVTMTRQLSQD
PDDPDWGTAFLELRSLTSDDTAVYFCTRGSYCTARDYYNWDFEHWGQGTPVTVSS
V_{L}
(SEQ ID NO: 10)
LTQSPGTLSLSPGETAIISCETSQYGSLAWYQQRPGQAPRLVIYSGSTRAAGIPDRESGSRWGPDYNLTISNLESGD
FGVYYCQQYEFFGQGTKVQVDIK
Heavy Chain
(SEQ ID NO: 18)
QVRLSQSGGQMKKPGDSMRISCSASGYEFINCPINWIRLAPGKRPEWMGWMKPRFGAVSYARQLQGRVTMTRQLSQD
PDDPDWGTAFLELRSLTSDDTAVYFCTRGSYCTARDYYNWDFEHWGQGTPVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 12)
LTQSPGTLSLSPGETAIISCETSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
VRC07-523LS.cv1
V_{H}
(SEQ ID NO: 1)
QVRLSQSGGQMKKPGDSMRISCRASGYEFINCPINWIRLAPGKRPEWMGWMKPRFGAVSYARQLQGRVTMTRQLSQD
PDDPDWGTAFLELRSLTSDDTAVYFCTRGKYCTARDYYNWDFEHWGQGTPVTVSS
V_{L}
(SEQ ID NO: 2)
LTQSPGTLSLSPGETAIISCRTSQYGSLAWYQQRPGQAPRLVIYSGSTRAAGIPDRFSGSRWGPDYNLTISNLESGD
FGVYYCQQYEFFGQGTKVQVDIK
Heavy Chain
(SEQ ID NO: 57)
QVRLSQSGGQMKKPGDSMRISCRASGYEFINCPINWIRLAPGKRPEWMGWMKPRFGAVSYARQLQGRVTMTRQLSQD
PDDPDWGTAFLELRSLTSDDTAVYFCTRGKYCTARDYYNWDFEHWGQGTPVTVSSASTKGPSVFPLAPSSESTSGGT
AALGCLVKDYFPEPVTVSWNSGALTSGVHTFPAVLQSSGLYSLSSVVTVPSSSLGTQTYICNVNHKPSNTKVDKEVE
PESCDKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 58)
LTQSPGTLSLSPGETAIISCRTSQYGSLAWYQQRPGQAPRLVIYSGSTRAAGIPDRESGSRWGPDYNLTISNLESGD
FGVYYCQQYEFFGQGTKVQVDIKRTVAAPSVFIFPPSDEQLESGTASVVCLLNNFYPREAEVQWKVDNALQSGNSQE
SVTEQDSKDSTYSLSSTLTLSKADYEEHEVYACEVTHQGLSSPVTKSFNRGEC
VRC07-523LS.cv34
V_{H}
(SEQ ID NO: 59)
QVRLSQSGGQMKKPGDSMRISCSASGYEFINCPINWIRLAPGKRPEWMGWMKPRFGAVSYARQLQGRVTMTRQLSQD
PDDPDWGTAFLELRSLTSDDTAVYFCTRGSYCTARDYYNWDFEHWGQGTPVTVSS
V_{L}
(SEQ ID NO: 10)
LTQSPGTLSLSPGETAIISCETSQYGSLAWYQQRPGQAPRLVIYSGSTRAAGIPDRFSGSRWGPDYNLTISNLESGD
FGVYYCQQYEFFGQGTKVQVDIK
Heavy Chain
(SEQ ID NO: 60)
QVRLSQSGGQMKKPGDSMRISCSASGYEFINCPINWIRLAPGKRPEWMGWMKPRFGAVSYARQLQGRVTMTRQLSQD
PDDPDWGTAFLELRSLTSDDTAVYFCTRGSYCTARDYYNWDFEHWGQGTPVTVSSASTKGPSVFPLAPSSESTSGGT
AALGCLVKDYFPEPVTVSWNSGALTSGVHTFPAVLQSSGLYSLSSVVTVPSSSLGTQTYICNVNHKPSNTKVDKEVE
PESCDKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 61)
LTQSPGTLSLSPGETAIISCETSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLESGTASVVCLLNNFYPREAEVQWKVDNALQSGNSQE
SVTEQDSKDSTYSLSSTLTLSKADYEEHEVYACEVTHQGLSSPVTKSFNRGEC
VRC07-523LS.ATS1
Heavy Chain
(SEQ ID NO: 3)
QVRLSQSGGQMKKPGDSMRISCRASGYEFINCPINWIRLAPGKRPEWMGWMKPRFGAVSYARQLQGRVTMTRQLSQD
PDDPDWGTAFLELRSLTSDDTAVYFCTRGKYCTARDYYNWDFEHWGQGTPVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 79)
LTQSPGTLSLSPGETAIISCRTSQYGSLAWYQQRPGQAPRLVIYSGSTRAAGIPDRESGSRWGPDYNLTISNLESGD
FGVYYCQQYEFFGQGTKVQVDIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGECggDPDNEAYEMPSEEGYQDYEPEA
VRC07-523LS.ATS4
Heavy Chain
(SEQ ID NO: 80)
QVRLSQSGGQMKKPGDSMRISCRASGYEFINCPINWIRLAPGKRPEWMGWMKPRFGAVSYARQLQGRVTMTRQLSQD
PDDPDWGTAFLELRSLTSDDTAVYFCTRGKYCTARDYYNWDFEHWGQGTPVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVLHEALHSHYTQKSLSLSPGK
ggDPDNEAYEMPSEEGYQDYEPEA
Light Chain
(SEQ ID NO: 81)
LTQSPGTLSLSPGETAIISCRTSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGECggDPDNEAYEMPSEEGYQDYEPEA
VRC07-523LS.ATS10
Heavy Chain
(SEQ ID NO: 82)
QVRLSQSGGQMKKPGDSMRISCSASGYEFINCPINWIRLAPGKRPEWMGWMKPRFGAVSYARQLQGRVTMTRQLSQD
PDDPDWGTAFLELRSLTSDDTAVYFCTRGSYCTARDYYNWDFEHWGQGTPVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVLHEALHSHYTQKSLSLSPGK
ggDPDNEAYEMPSEEGYQDYEPEA
Light Chain
(SEQ ID NO: 83)
LTQSPGTLSLSPGETAIISCETSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGECggDPDNEAYEMPSEEGYQDYEPEA
N6LS-03FR3
V_{H}
(SEQ ID NO: 19)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSA
V_{L}
(SEQ ID NO: 20)
YIHVTQSPSSLSVSIGDRVTINCQTSQGVGSDLHWYQHKPGRAPKLLIHHTSSVEDGVPSRFSGSGFHTSFNLTISD
LQADDIATYYCQVLQFFGRGSRLHIK
Heavy Chain
(SEQ ID NO: 21)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGK
Light Chain
(SEQ ID NO: 22)
YIHVTQSPSSLSVSIGDRVTINCQTSQGVGSDLHWYQHKPGRAPKLLIHHTSSVEDGVPSRFSGSGEHTSENLTISD
LQADDIATYYCQVLQFFGRGSRLHIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
N6LS.C1
V_{H}
(SEQ ID NO: 23)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIDGLKPDDTAVYYCARDRSYGDSSWALDAWGQGTTVVVSA
V_{L}
(SEQ ID NO: 24)
YIHVTQSPSSLSVSIGDDVTINCQTSQGVGSDLHWYQHKPGRAPKLLIHHTSSVEDGVPSRFSGSGEHTSENLTISD
LQADDIATYYCQVLQFFGRGSRLHIK
Heavy Chain
(SEQ ID NO: 25)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIDGLKPDDTAVYYCARDRSYGDSSWALDAWGQGTTVVVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGK
Light Chain
(SEQ ID NO: 26)
YIHVTQSPSSLSVSIGDDVTINCQTSQGVGSDLHWYQHKPGRAPKLLIHHTSSVEDGVPSRFSGSGFHTSFNLTISD
LQADDIATYYCQVLQFFGRGSRLHIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
N6LS.C15
V_{H}
(SEQ ID NO: 19)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSA
V_{L}
(SEQ ID NO: 27)
YIHVTQSPSSLSVSIGDEVTINCQTSQGVGSDLHWYQHKPGEAPELLIHHTSSVEDGVPSRFSGSGEHTSENLTISD
LQADDIATYYCQVLQFFGRGSRLHIK
Heavy Chain
(SEQ ID NO: 21)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGK
Light Chain
(SEQ ID NO: 28)
YIHVTQSPSSLSVSIGDEVTINCQTSQGVGSDLHWYQHKPGEAPELLIHHTSSVEDGVPSRFSGSGEHTSFNLTISD
LQADDIATYYCQVLQFFGRGSRLHIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
N6LS.C30
V_{H}
(SEQ ID NO: 29)
RAHLVQSGTAMKEPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSA
V_{L}
(SEQ ID NO: 30)
YIHVTQSPSSLSVSIGDEVTINCQTSQGVGSDLHWYQHKPGRAPKLLIHHTSSVEDGVPSRFSGSGFHTSFNLTISD
LQADDIATYYCQVLQFFGRGSRLHIK
Heavy Chain
(SEQ ID NO: 31)
RAHLVQSGTAMKEPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKENWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGK
Light Chain
(SEQ ID NO: 32)
YIHVTQSPSSLSVSIGDEVTINCQTSQGVGSDLHWYQHKPGRAPKLLIHHTSSVEDGVPSRFSGSGFHTSFNLTISD
LQADDIATYYCQVLQFFGRGSRLHIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
N6LS.C35
V_{H}
(SEQ ID NO: 29)
RAHLVQSGTAMKEPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSA
V_{L}
(SEQ ID NO: 27)
YIHVTQSPSSLSVSIGDEVTINCQTSQGVGSDLHWYQHKPGEAPELLIHHTSSVEDGVPSRFSGSGEHTSENLTISD
LQADDIATYYCQVLQFFGRGSRLHIK
Heavy Chain
(SEQ ID NO: 31)
RAHLVQSGTAMKEPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGK
Light Chain
(SEQ ID NO: 28)
YIHVTQSPSSLSVSIGDEVTINCQTSQGVGSDLHWYQHKPGEAPELLIHHTSSVEDGVPSRFSGSGFHTSFNLTISD
LQADDIATYYCQVLQFFGRGSRLHIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
N6LS.C47
V_{H}
(SEQ ID NO: 19)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSA
V_{L}
(SEQ ID NO: 33)
YIHVTQSPSSLSVSIGDQVTINCQTSQGVGSDLHWYQHKPGRAPKLLIHHTSSVEDGVPSRFSGSGEHTSENLTISD
LQADDIATYYCQVLQFFGRGSRLHIK
Heavy Chain
(SEQ ID NO: 21)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKENWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGK
Light Chain
(SEQ ID NO: 34)
YIHVTQSPSSLSVSIGDQVTINCQTSQGVGSDLHWYQHKPGRAPKLLIHHTSSVEDGVPSRFSGSGFHTSFNLTISD
LQADDIATYYCQVLQFFGRGSRLHIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
N6LS.C49
V_{H}
(SEQ ID NO: 35)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIQGLKPDDTAVYYCARDRSYGDSSWALDAWGQGTTVVVSA
V_{L}
(SEQ ID NO: 33)
YIHVTQSPSSLSVSIGDQVTINCQTSQGVGSDLHWYQHKPGRAPKLLIHHTSSVEDGVPSRFSGSGFHTSFNLTISD
LQADDIATYYCQVLQFFGRGSRLHIK
Heavy Chain
(SEQ ID NO: 36)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIQGLKPDDTAVYYCARDRSYGDSSWALDAWGQGTTVVVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGK
Light Chain
(SEQ ID NO: 34)
YIHVTQSPSSLSVSIGDQVTINCQTSQGVGSDLHWYQHKPGRAPKLLIHHTSSVEDGVPSRFSGSGFHTSFNLTISD
LQADDIATYYCQVLQFFGRGSRLHIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
N6LS.C51
V_{H}
(SEQ ID NO: 37)
RAHLVQSGTAMKQPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSA
V_{L}
(SEQ ID NO: 33)
YIHVTQSPSSLSVSIGDQVTINCQTSQGVGSDLHWYQHKPGRAPKLLIHHTSSVEDGVPSRFSGSGEHTSENLTISD
LQADDIATYYCQVLQFFGRGSRLHIK
Heavy Chain
(SEQ ID NO: 38)
RAHLVQSGTAMKQPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGK
Light Chain
(SEQ ID NO: 34)
YIHVTQSPSSLSVSIGDQVTINCQTSQGVGSDLHWYQHKPGRAPKLLIHHTSSVEDGVPSRFSGSGFHTSFNLTISD
LQADDIATYYCQVLQFFGRGSRLHIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
N6LS.C58
V_{H}
(SEQ ID NO: 39)
RAHLVQSGTAMKKPGASVRVSCQTSGYTFTAHILFWFRQAPGQGLEWVGWIKPQYGAVNFGGGFRDRVTLTRQLSQD
PDDPDWGIAYMDIRGLKPDDTAVYYCARDRSYGDSSWALDAWGQGTTVVVSA
V_{L}
(SEQ ID NO: 33)
YIHVTQSPSSLSVSIGDQVTINCQTSQGVGSDLHWYQHKPGRAPKLLIHHTSSVEDGVPSRFSGSGFHTSFNLTISD
LQADDIATYYCQVLQFFGRGSRLHIK
Heavy Chain
(SEQ ID NO: 40)
RAHLVQSGTAMKKPGASVRVSCQTSGYTFTAHILFWFRQAPGQGLEWVGWIKPQYGAVNFGGGFRDRVTLTRQLSQD
PDDPDWGIAYMDIRGLKPDDTAVYYCARDRSYGDSSWALDAWGQGTTVVVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGK
Light Chain
(SEQ ID NO: 34)
YIHVTQSPSSLSVSIGDQVTINCQTSQGVGSDLHWYQHKPGRAPKLLIHHTSSVEDGVPSRFSGSGFHTSFNLTISD
LQADDIATYYCQVLQFFGRGSRLHIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
N6LS.cv1
V_{H}
(SEQ ID NO: 19)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSA
V_{L}
(SEQ ID NO: 20)
YIHVTQSPSSLSVSIGDRVTINCQTSQGVGSDLHWYQHKPGRAPKLLIHHTSSVEDGVPSRFSGSGFHTSFNLTISD
LQADDIATYYCQVLQFFGRGSRLHIK
Heavy Chain
(SEQ ID NO: 62)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSAASTKGPSVFPLAPSSESTSGGTAAL
GCLVKDYFPEPVTVSWNSGALTSGVHTFPAVLQSSGLYSLSSVVTVPSSSLGTQTYICNVNHKPSNTKVDKEVEPES
CDKTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGK
Light Chain
(SEQ ID NO: 63)
YIHVTQSPSSLSVSIGDRVTINCQTSQGVGSDLHWYQHKPGRAPKLLIHHTSSVEDGVPSRFSGSGFHTSFNLTISD
LQADDIATYYCQVLQFFGRGSRLHIKRTVAAPSVFIFPPSDEQLESGTASVVCLLNNFYPREAEVQWKVDNALQSGN
SQESVTEQDSKDSTYSLSSTLTLSKADYEEHEVYACEVTHQGLSSPVTKSFNRGEC
N6LS.cv49
V_{H}
(SEQ ID NO: 35)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIQGLKPDDTAVYYCARDRSYGDSSWALDAWGQGTTVVVSA
V_{L}
(SEQ ID NO: 33)
YIHVTQSPSSLSVSIGDQVTINCQTSQGVGSDLHWYQHKPGRAPKLLIHHTSSVEDGVPSRFSGSGFHTSFNLTISD
LQADDIATYYCQVLQFFGRGSRLHIK
Heavy Chain
(SEQ ID NO: 64)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIQGLKPDDTAVYYCARDRSYGDSSWALDAWGQGTTVVVSAASTKGPSVFPLAPSSESTSGGTAAL
GCLVKDYFPEPVTVSWNSGALTSGVHTFPAVLQSSGLYSLSSVVTVPSSSLGTQTYICNVNHKPSNTKVDKEVEPES
CDKTHTCPPCPAPELLGGPSVFLFPPKPKDTLMISRTPEVTCVVVDVSHEDPEVKENWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGK
Light Chain
(SEQ ID NO: 97)
YIHVTQSPSSLSVSIGDQVTINCQTSQGVGSDLHWYQHKPGRAPKLLIHHTSSVEDGVPSRFSGSGEHTSENLTISD
LQADDIATYYCQVLQFFGRGSRLHIKRTVAAPSVFIFPPSDEQLESGTASVVCLLNNFYPREAEVQWKVDNALQSGN
SQESVTEQDSKDSTYSLSSTLTLSKADYEEHEVYACEVTHQGLSSPVTKSFNRGEC
N6LS.ATS4
Heavy Chain
(SEQ ID NO: 84)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIRGLKPDDTAVYYCARDRSYGDSSWALDAWGQGTTVVVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKENWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGKggD
PDNEAYEMPSEEGYQDYEPEA
Light Chain
(SEQ ID NO: 85)
YIHVTQSPSSLSVSIGDRVTINCQTSQGVGSDLHWYQHKPGRAPKLLIHHTSSVEDGVPSRFSGSGFHTSFNLTISD
LQADDIATYYCQVLQFFGRGSRLHIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGECggDPDNEAYEMPSEEGYQDYE
PEA
N6LS.ATS8
Heavy Chain
(SEQ ID NO: 86)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIDGLKPDDTAVYYCARDRSYGDSSWALDAWGQGTTVVVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKENWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGKggD
PDNEAYEMPSEEGYQDYEPEA
Light Chain
(SEQ ID NO: 87)
YIHVTQSPSSLSVSIGDQVTINCQTSQGVGSDLHWYQHKPGRAPKLLIHHTSSVEDGVPSRFSGSGFHTSFNLTISD
LQADDIATYYCQVLQFFGRGSRLHIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGECggDPDNEAYEMPSEEGYQDYE
PEA
N6LS.ATS9
Heavy Chain
(SEQ ID NO: 88)
RAHLVQSGTAMKKPGASVRVSCQTSGYTFTAHILFWFRQAPGRGLEWVGWIKPQYGAVNFGGGFRDRVTLTRQLSQD
PDDPDWGIAYMDIQGLKPDDTAVYYCARDRSYGDSSWALDAWGQGTTVVVSAASTKGPSVFPLAPSSESTSGGTAAL
GCLVKDYFPEPVTVSWNSGALTSGVHTFPAVLQSSGLYSLSSVVTVPSSSLGTQTYICNVNHKPSNTKVDKEVEPES
CDKTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVLHEALHSHYTQKSLSLSPGKggD
PDNEAYEMPSEEGYQDYEPEA
Light Chain
(SEQ ID NO: 89)
YIHVTQSPSSLSVSIGDQVTINCQTSQGVGSDLHWYQHKPGRAPKLLIHHTSSVEDGVPSRFSGSGFHTSFNLTISD
LQADDIATYYCQVLQFFGRGSRLHIKRTVAAPSVFIFPPSDEQLESGTASVVCLLNNFYPREAEVQWKVDNALQSGN
SQESVTEQDSKDSTYSLSSTLTLSKADYEEHEVYACEVTHQGLSSPVTKSFNRGECggDPDNEAYEMPSEEGYQDYE
PEA
10E8v4-5RLS
V_{H}
(SEQ ID NO: 41)
EVRLRESGGGLVKPGGSLRLSCSASGFDFDNAWMTWVRQPPGKGLEWVGRITGPGEGWSVDYAESVKGRFTISRDNT
KNTLYLEMNNVRTEDTGYYFCARTGKYYDFWSGYPPGEEYFQDWGQGTLVIVSS
V_{L}
(SEQ ID NO: 42)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVL
Heavy Chain
(SEQ ID NO: 43)
EVRLRESGGGLVKPGGSLRLSCSASGFDFDNAWMTWVRQPPGKGLEWVGRITGPGEGWSVDYAESVKGRFTISRDNT
KNTLYLEMNNVRTEDTGYYFCARTGKYYDFWSGYPPGEEYFQDWGQGTLVIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKENWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 44)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
10E8v4-5RLS. C12
V_{H}
(SEQ ID NO: 45)
EVRLRESGGGLVEPGGSLRLSCSASGFDFDNAWMTWVRQPPGEGLEWVGRITGPGEGWSVDYAESVKGRFTISRDNT
KNTLYLEMNNVRTEDTGYYFCARTGKYYDFWSGYPPGEEYFQDWGQGTLVIVSS
V_{L}
(SEQ ID NO: 42)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVL
Heavy Chain
(SEQ ID NO: 46)
EVRLRESGGGLVEPGGSLRLSCSASGFDFDNAWMTWVRQPPGEGLEWVGRITGPGEGWSVDYAESVKGRFTISRDNT
KNTLYLEMNNVRTEDTGYYFCARTGKYYDFWSGYPPGEEYFQDWGQGTLVIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKENWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 44)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
10E8v4-5RLS.C13
V_{H}
(SEQ ID NO: 45)
EVRLRESGGGLVEPGGSLRLSCSASGFDFDNAWMTWVRQPPGEGLEWVGRITGPGEGWSVDYAESVKGRFTISRDNT
KNTLYLEMNNVRTEDTGYYFCARTGKYYDFWSGYPPGEEYFQDWGQGTLVIVSS
V_{L}
(SEQ ID NO: 47)
ASELTQDPAVSVALEQTVTITCRGDSLRSHYASWYQKKPGQAPVLLFYGKNNRPSGIPDRFSGSASGNRASLTITGA
QAEDEADYYCSSRDKSGSRLSVFGGGTKLTVL
Heavy Chain
(SEQ ID NO: 46)
EVRLRESGGGLVEPGGSLRLSCSASGFDFDNAWMTWVRQPPGEGLEWVGRITGPGEGWSVDYAESVKGRFTISRDNT
KNTLYLEMNNVRTEDTGYYFCARTGKYYDFWSGYPPGEEYFQDWGQGTLVIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKENWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 48)
ASELTQDPAVSVALEQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
10E8v4-5RLS. C14
V_{H}
(SEQ ID NO: 45)
EVRLRESGGGLVEPGGSLRLSCSASGFDFDNAWMTWVRQPPGEGLEWVGRITGPGEGWSVDYAESVKGRFTISRDNT
KNTLYLEMNNVRTEDTGYYFCARTGKYYDFWSGYPPGEEYFQDWGQGTLVIVSS
V_{L}
(SEQ ID NO: 49)
ASELTQDPAVSVALKQTVTITCRGDSLRSHYASWYQKKPGQAPVLLFYGKNNRPSGIPDRFSGSASGNRASLTITGA
QAEDEADYYCSSRDESGSRLSVFGGGTKLTVL
Heavy Chain
(SEQ ID NO: 46)
EVRLRESGGGLVEPGGSLRLSCSASGFDFDNAWMTWVRQPPGEGLEWVGRITGPGEGWSVDYAESVKGRFTISRDNT
KNTLYLEMNNVRTEDTGYYFCARTGKYYDFWSGYPPGEEYFQDWGQGTLVIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 50)
ASELTQDPAVSVALKQTVTITCRGDSLRSHYASWYQKKPGQAPVLLFYGKNNRPSGIPDRFSGSASGNRASLTITGA
QAEDEADYYCSSRDESGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
10E8v4-5RLS.C15
V_{H}
(SEQ ID NO: 51)
EVELRESGGGLVKPGGSLRLSCSASGEDEDNAWMTWVRQPPGKGLEWVGRITGPGEGWSVDYAESVKGRFTISRDNT
KNTLYLEMNNVETEDTGYYFCARTGKYYDFWSGYPPGEEYFQDWGQGTLVIVSS
V_{L}
(SEQ ID NO: 42)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVL
Heavy Chain
(SEQ ID NO: 52)
EVELRESGGGLVKPGGSLRLSCSASGFDEDNAWMTWVRQPPGKGLEWVGRITGPGEGWSVDYAESVKGRFTISRDNT
KNTLYLEMNNVETEDTGYYFCARTGKYYDFWSGYPPGEEYFQDWGQGTLVIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 44)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
10E8v4-5RLS.C18
V_{H}
(SEQ ID NO: 53)
EVELRESGGGLVKPGGSLRLSCSASGFDFDNAWMTWVRQPPGEGLEWVGRITGPGEGWSVDYAESVKGRFTISRDNT
KNTLYLEMNNVETEDTGYYFCARTGKYYDFWSGYPPGEEYFQDWGQGTLVIVSS
V_{L}
(SEQ ID NO: 42)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVL
Heavy Chain
(SEQ ID NO: 54)
EVELRESGGGLVKPGGSLRLSCSASGFDFDNAWMTWVRQPPGEGLEWVGRITGPGEGWSVDYAESVKGRFTISRDNT
KNTLYLEMNNVETEDTGYYFCARTGKYYDFWSGYPPGEEYFQDWGQGTLVIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKENWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 44)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
10E8v4-5RLS.C24
V_{H}
(SEQ ID NO: 55)
EVELRESGGGLVEPGGSLRLSCSASGFDFDNAWMTWVRQPPGEGLEWVGRITGPGEGWSVDYAESVKGRFTISRDNT
KNTLYLEMNNVETEDTGYYFCARTGKYYDFWSGYPPGEEYFQDWGQGTLVIVSS
V_{L}
(SEQ ID NO: 42)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVL
Heavy Chain
(SEQ ID NO: 56)
EVELRESGGGLVEPGGSLRLSCSASGFDEDNAWMTWVRQPPGEGLEWVGRITGPGEGWSVDYAESVKGRFTISRDNT
KNTLYLEMNNVETEDTGYYFCARTGKYYDFWSGYPPGEEYFQDWGQGTLVIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 44)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
10E8v4-5RLS.C27
V_{H}
(SEQ ID NO: 65)
EVRLRESGGGLVSPGGSLRLSCSASGFDFDNAWMTWVRQPPGSGLEWVGRITGPGEGWSVDYAESVKGRFTISRDNT
KNTLYLEMNNVRTEDTGYYFCARTGKYYDFWSGYPPGEEYFQDWGQGTLVIVSS
V_{L}
(SEQ ID NO: 66)
ASELTQDPAVSVALSQTVTITCRGDSLRSHYASWYQKKPGQAPVLLFYGKNNRPSGIPDRFSGSASGNRASLTITGA
QAEDEADYYCSSRDKSGSRLSVFGGGTKLTVL
Heavy Chain
(SEQ ID NO: 67)
EVRLRESGGGLVSPGGSLRLSCSASGFDFDNAWMTWVRQPPGSGLEWVGRITGPGEGWSVDYAESVKGRFTISRDNT
KNTLYLEMNNVRTEDTGYYFCARTGKYYDFWSGYPPGEEYFQDWGQGTLVIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKENWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 68)
ASELTQDPAVSVALSQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
10E8v4-5RLS.C30
V_{H}
(SEQ ID NO: 65)
EVRLRESGGGLVSPGGSLRLSCSASGFDFDNAWMTWVRQPPGSGLEWVGRITGPGEGWSVDYAESVKGRFTISRDNT
KNTLYLEMNNVRTEDTGYYFCARTGKYYDFWSGYPPGEEYFQDWGQGTLVIVSS
V_{L}
(SEQ ID NO: 69)
ASELTQDPAVSVALKQTVTITCRGDSLRSHYASWYQKKPGQAPVLLFYGKNNRPSGIPDRFSGSASGNRASLTITGA
QAEDEADYYCSSRDQSGSRLSVFGGGTKLTVL
Heavy Chain
(SEQ ID NO: 67)
EVRLRESGGGLVSPGGSLRLSCSASGFDEDNAWMTWVRQPPGSGLEWVGRITGPGEGWSVDYAESVKGRFTISRDNT
KNTLYLEMNNVRTEDTGYYFCARTGKYYDFWSGYPPGEEYFQDWGQGTLVIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKENWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 70)
ASELTQDPAVSVALKQTVTITCRGDSLRSHYASWYQKKPGQAPVLLFYGKNNRPSGIPDRFSGSASGNRASLTITGA
QAEDEADYYCSSRDQSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
10E8v4.cc11
V_{H}
(SEQ ID NO: 41)
EVRLRESGGGLVKPGGSLRLSCSASGFDFDNAWMTWVRQPPGKGLEWVGRITGPGEGWSVDYAESVKGRFTISRDNT
KNTLYLEMNNVRTEDTGYYFCARTGKYYDFWSGYPPGEEYFQDWGQGTLVIVSS
V_{L}
(SEQ ID NO: 42)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVL
Heavy Chain
(SEQ ID NO: 43)
EVRLRESGGGLVKPGGSLRLSCSASGFDFDNAWMTWVRQPPGKGLEWVGRITGPGEGWSVDYAESVKGRFTISRDNT
KNTLYLEMNNVRTEDTGYYFCARTGKYYDFWSGYPPGEEYFQDWGQGTLVIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKENWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 71)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPEAAPSVTLFPPSSEELQANEATLVCLISDFYPGAVTVAWKAD
SSPVEAGVETTTPSEQSNNKYAASSYLSLTPEQWESHESYSCQVTHEGSTVEKTVAPTECS
10E8v4.cc16
V_{H}
(SEQ ID NO: 41)
EVRLRESGGGLVKPGGSLRLSCSASGFDFDNAWMTWVRQPPGKGLEWVGRITGPGEGWSVDYAESVKGRFTISRDNT
KNTLYLEMNNVRTEDTGYYFCARTGKYYDFWSGYPPGEEYFQDWGQGTLVIVSS
V_{L}
(SEQ ID NO: 42)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVL
Heavy Chain
(SEQ ID NO: 72)
EVRLRESGGGLVKPGGSLRLSCSASGFDFDNAWMTWVRQPPGKGLEWVGRITGPGEGWSVDYAESVKGRFTISRDNT
KNTLYLEMNNVRTEDTGYYFCARTGKYYDFWSGYPPGEEYFQDWGQGTLVIVSSEETEGPSVFPLAPSSESTEGGTA
ALGCLVKDYFPEPVTVSWNSGELTSGVHTFPAVLQSSGLYSLSSVVTVPSSSLGEETYICNVNHEPSNTEVDKEVEP
ESCDKTHTCPPCPAPELLGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 44)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
10E8v4-5RLS.cv1
V_{H}
(SEQ ID NO: 41)
EVRLRESGGGLVKPGGSLRLSCSASGFDFDNAWMTWVRQPPGKGLEWVGRITGPGEGWSVDYAESVKGRFTISRDNT
KNTLYLEMNNVRTEDTGYYFCARTGKYYDFWSGYPPGEEYFQDWGQGTLVIVSS
V_{L}
(SEQ ID NO: 42)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVL
Heavy Chain
(SEQ ID NO: 73)
EVRLRESGGGLVKPGGSLRLSCSASGFDEDNAWMTWVRQPPGKGLEWVGRITGPGEGWSVDYAESVKGRFTISRDNT
KNTLYLEMNNVRTEDTGYYFCARTGKYYDFWSGYPPGEEYFQDWGQGTLVIVSSASTKGPSVFPLAPSSESTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKENWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 74)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPKAAPSVTLFPPSSEELQANKATLVCLISDFYPGAVTVAWKAD
SSPVKAGVETTTPSEQSNNKYAASSYLSLTPEQWESHESYSCQVTHEGSTVEETVAPTECS
10E8v4-5RLS. cv30
V_{H}
(SEQ ID NO: 65)
EVRLRESGGGLVSPGGSLRLSCSASGFDFDNAWMTWVRQPPGSGLEWVGRITGPGEGWSVDYAESVKGRFTISRDNT
KNTLYLEMNNVRTEDTGYYFCARTGKYYDFWSGYPPGEEYFQDWGQGTLVIVSS
V_{L}
(SEQ ID NO: 69)
ASELTQDPAVSVALKQTVTITCRGDSLRSHYASWYQKKPGQAPVLLFYGKNNRPSGIPDRFSGSASGNRASLTITGA
QAEDEADYYCSSRDQSGSRLSVFGGGTKLTVL
Heavy Chain
(SEQ ID NO: 75)
EVRLRESGGGLVSPGGSLRLSCSASGFDFDNAWMTWVRQPPGSGLEWVGRITGPGEGWSVDYAESVKGRFTISRDNT
KNTLYLEMNNVRTEDTGYYFCARTGKYYDFWSGYPPGEEYFQDWGQGTLVIVSSASTKGPSVFPLAPSSESTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKENWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGK
Light Chain
(SEQ ID NO: 76)
ASELTQDPAVSVALKQTVTITCRGDSLRSHYASWYQKKPGQAPVLLFYGKNNRPSGIPDRESGSASGNRASLTITGA
QAEDEADYYCSSRDQSGSRLSVFGGGTKLTVLSQPKAAPSVTLFPPSSEELQANKATLVCLISDFYPGAVTVAWKAD
SSPVKAGVETTTPSEQSNNKYAASSYLSLTPEQWESHESYSCQVTHEGSTVEETVAPTECS
10E8.ATS4
Heavy Chain
(SEQ ID NO: 90)
EVRLRESGGGLVKPGGSLRLSCSASGFDFDNAWMTWVRQPPGKGLEWVGRITGPGEGWSVDYAESVKGRFTISRDNT
KNTLYLEMNNVRTEDTGYYFCARTGKYYDFWSGYPPGEEYFQDWGQGTLVIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGKg
gDPDNEAYEMPSEEGYQDYEPEA
Light Chain
(SEQ ID NO: 91)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECSggDPDNEAYEMPSEEG
YQDYEPEA
10E8.ATS11
Heavy Chain
(SEQ ID NO: 92)
EVRLRESGGGLVSPGGSLRLSCSASGFDFDNAWMTWVRQPPGSGLEWVGRITGPGEGWSVDYAESVKGRFTISRDNT
KNTLYLEMNNVRTEDTGYYFCARTGKYYDFWSGYPPGEEYFQDWGQGTLVIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKFNWYVDGVEVHNAKTKPREE
QYNSTYRVVSVLIVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGKg
gDPDNEAYEMPSEEGYQDYEPEA
Light Chain
(SEQ ID NO: 93)
ASELTQDPAVSVALSQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECSggDPDNEAYEMPSEEG
YQDYEPEA
10E8.ATS13
V_{H}
(SEQ ID NO: 98)
EVRLRESGGGLVSPGGSLRLSCSASGEDEDNAWMTWVRQPPGSGLEWVGRITGPGEGWSVDYAESVKGRFTISRDNT
KNTLYLEMNNVRTEDTGYYFCARTGKYYDFWFGYPPGEEYFQDWGQGTLVIVSS
Heavy Chain
(SEQ ID NO: 94)
EVRLRESGGGLVSPGGSLRLSCSASGFDFDNAWMTWVRQPPGSGLEWVGRITGPGEGWSVDYAESVKGRFTISRDNT
KNTLYLEMNNVRTEDTGYYFCARTGKYYDFWFGYPPGEEYFQDWGQGTLVIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKENWYVDGVEVHNAKTKPREE
QYNSTYRVVSVLIVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVLHEALHSHYTQKSLSLSPGKg
gDPDNEAYEMPSEEGYQDYEPEA
Light Chain
(SEQ ID NO: 93)
ASELTQDPAVSVALSQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECSggDPDNEAYEMPSEEG
YQDYEPEA
VRC01.23LS.cv1
V_{H}
(SEQ ID NO: 77)
QVQLVQSGGQMKKPGESMRISCRASGYEFIDCTLNWIRLAPGKRPEWMGWLKPRWGAVNYARPLQGRVTMTRQLSQD
PDDPDWGTAFLELRSLTVDDTAVYFCTRGKNCDYNWDFEHWGRGTPVIVSS
V_{L}
(SEQ ID NO: 2)
LTQSPGTLSLSPGETAIISCRTSQYGSLAWYQQRPGQAPRLVIYSGSTRAAGIPDRFSGSRWGPDYNLTISNLESGD
FGVYYCQQYEFFGQGTKVQVDIK
Heavy Chain
(SEQ ID NO: 78)
QVQLVQSGGQMKKPGESMRISCRASGYEFIDCTLNWIRLAPGKRPEWMGWLKPRWGAVNYARPLQGRVTMTRQLSQD
PDDPDWGTAFLELRSLTVDDTAVYFCTRGKNCDYNWDFEHWGRGTPVIVSSASTKGPSVFPLAPSSESTSGGTAALG
CLVKDYFPEPVTVSWNSGALTSGVHTFPAVLQSSGLYSLSSVVTVPSSSLGTQTYICNVNHKPSNTKVDKEVEPESC
DKTHTCPPCPAPELLGGPSVFLFPPKPKDTLMISRTPEVTCVVVDVSHEDPEVKENWYVDGVEVHNAKTKPREEQYN
STYRVVSVLTVLHQDWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVLHEALHSHYTQKSLSLSPGK
Light Chain
(SEQ ID NO: 58)
LTQSPGTLSLSPGETAIISCRTSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLESGTASVVCLLNNFYPREAEVQWKVDNALQSGNSQE
SVTEQDSKDSTYSLSSTLTLSKADYEEHEVYACEVTHQGLSSPVTKSFNRGEC
VRC01.23LS.cv34
V_{H}
(SEQ ID NO: 59)
QVQLVQSGGQMKKPGESMRISCSASGYEFIDCTLNWIRLAPGKRPEWMGWLKPRWGAVNYARPLQGRVTMTRQLSQD
PDDPDWGTAFLELRSLTVDDTAVYFCTRGSNCDYNWDFEHWGRGTPVIVSS
V_{L}
(SEQ ID NO: 10)
LTQSPGTLSLSPGETAIISCETSQYGSLAWYQQRPGQAPRLVIYSGSTRAAGIPDRFSGSRWGPDYNLTISNLESGD
FGVYYCQQYEFFGQGTKVQVDIK
Heavy Chain
(SEQ ID NO: 60)
QVQLVQSGGQMKKPGESMRISCSASGYEFIDCTLNWIRLAPGKRPEWMGWLKPRWGAVNYARPLQGRVTMTRQLSQD
PDDPDWGTAFLELRSLTVDDTAVYFCTRGSNCDYNWDFEHWGRGTPVIVSSASTKGPSVFPLAPSSESTSGGTAALG
CLVKDYFPEPVTVSWNSGALTSGVHTFPAVLQSSGLYSLSSVVTVPSSSLGTQTYICNVNHKPSNTKVDKEVEPESC
DKTHTCPPCPAPELLGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVLHEALHSHYTQKSLSLSPGK
Light Chain
(SEQ ID NO: 61)
LTQSPGTLSLSPGETAIISCETSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLESGTASVVCLLNNFYPREAEVQWKVDNALQSGNSQE
SVTEQDSKDSTYSLSSTLTLSKADYEEHEVYACEVTHQGLSSPVTKSFNRGEC
VRC01.23LS.ATS1
Heavy Chain
(SEQ ID NO: 95)
QVQLVQSGGQMKKPGESMRISCRASGYEFIDCTLNWIRLAPGKRPEWMGWLKPRWGAVNYARPLQGRVTMTRQLSQD
PDDPDWGTAFLELRSLTVDDTAVYFCTRGKNCDYNWDFEHWGRGTPVIVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKENWYVDGVEVHNAKTKPREEQYN
STYRVVSVLTVLHQDWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVLHEALHSHYTQKSLSLSPGK
Light Chain
(SEQ ID NO: 79)
LTQSPGTLSLSPGETAIISCRTSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGECggDPDNEAYEMPSEEGYQDYEPEA
VRC01.23LS.ATS4
Heavy Chain
(SEQ ID NO: 96)
QVQLVQSGGQMKKPGESMRISCRASGYEFIDCTLNWIRLAPGKRPEWMGWLKPRWGAVNYARPLQGRVTMTRQLSQD
PDDPDWGTAFLELRSLTVDDTAVYFCTRGKNCDYNWDFEHWGRGTPVIVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKFNWYVDGVEVHNAKTKPREEQYN
STYRVVSVLIVLHQDWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVLHEALHSHYTQKSLSLSPGKggDP
DNEAYEMPSEEGYQDYEPEA
Light Chain
(SEQ ID NO: 79)
LTQSPGTLSLSPGETAIISCRISQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGECggDPDNEAYEMPSEEGYQDYEPEA
VRC01.23LS.ATS5
Heavy Chain
(SEQ ID NO: 96)
QVQLVQSGGQMKKPGESMRISCRASGYEFIDCTLNWIRLAPGKRPEWMGWLKPRWGAVNYARPLQGRVTMTRQLSQD
PDDPDWGTAFLELRSLTVDDTAVYFCTRGKNCDYNWDFEHWGRGTPVIVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKENWYVDGVEVHNAKTKPREEQYN
STYRVVSVLTVLHQDWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVLHEALHSHYTQKSLSLSPGKggDP
DNEAYEMPSEEGYQDYEPEA
Light Chain
(SEQ ID NO: 83)
LTQSPGTLSLSPGETAIISCETSQYGSLAWYQQRPGQAPRLVIYSGSTRAAGIPDRFSGSRWGPDYNLTISNLESGD
FGVYYCQQYEFFGQGTKVQVDIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGECggDPDNEAYEMPSEEGYQDYEPEA

It will be apparent that the precise details of the methods or compositions described may be varied or modified without departing from the spirit of the described implementations. We claim all such modifications and variations that fall within the scope and spirit of the claims below.

1. A monoclonal antibody, comprising a heavy chain variable region (V_{H}) and a light chain variable region (V_{L}) comprising amino acid sequences set forth as any one of:
(1) SEQ ID NOs: 59 and 10, respectively (VRC07-523LS.cv34);
(2) SEQ ID NOs: 13 and 14, respectively (VRC07-523LS.v26);
(3) SEQ ID NOs: 1 and 5, respectively (VRC07-523LS.v11);
(4) SEQ ID NOs: 7 and 5, respectively (VRC07-523LS.v14);
(5) SEQ ID NOs: 9 and 10, respectively (VRC07-523LS.v21);
(6) SEQ ID NOs: 17 and 14, respectively (VRC07-523LS.v32);
(7) SEQ ID NOs: 17 and 10, respectively (VRC07-523LS.v34);
(8) SEQ ID NOs: 23 and 24, respectively (N6LS.C1);
(9) SEQ ID NOs: 19 and 27, respectively (N6LS.15);
(10) SEQ ID NOs: 29 and 30, respectively (N6LS.30);
(11) SEQ ID NOs: 29 and 27, respectively (N6LS.35);
(12) SEQ ID NOs: 19 and 33, respectively (N6LS.47);
(13) SEQ ID NOs: 35 and 33, respectively (N6LS.49);
(14) SEQ ID NOs: 37 and 33, respectively (N6LS.51);
(15) SEQ ID NOs: 39 and 33, respectively (N6LS.58);
(16) SEQ ID NO: 23 and 33, respectively (N6LS.ATS8);
(17) SEQ ID NOs: 45 and 42, respectively (10E8v4-5RLS.C12);
(18) SEQ ID NOs: 45 and 47, respectively (10E8v4-5RLS.C13);
(19) SEQ ID NOs: 45 and 49, respectively (10E8v4-5RLS.C14);
(20) SEQ ID NOs: 51 and 52, respectively (10E8v4-5RLS.C15);
(21) SEQ ID NOs: 53 and 42, respectively (10E8v4-5RLS.C18);
(22) SEQ ID NOs: 55 and 42, respectively (10E8v4-5RLS.C24);
(23) SEQ ID NO: 65 and 66, respectively (10E8v4-5RLS.C27);
(24) SEQ ID NO: 65 and 69, respectively (10E8v4-5RLS.C30);
(25) SEQ ID NO: 98 and 66, respectively (10E8.ATS13);
(26) SEQ ID NO: 77 and 2, respectively (VRC01.23LS.cv1);
(27) SEQ ID NO: 59 and 10, respectively (VRC01.23LS.cv34); or
(28) SEQ ID NO: 77 and 10, respectively (VRC01.23LS.ATS5); and
wherein the antibody specifically binds to HIV-1 Env and neutralizes HIV-1.

2. The monoclonal antibody of claim 1, wherein the antibody is an IgG, IgM or IgA.

3. The monoclonal antibody of claim 1, wherein the antibody is an IgG1 and comprises:
a heavy chain comprising K129E, K222E, and K228E substitutions according to the Kabat numbering system; and/or
a kappa light chain comprising K126E, K145E, K188E, K190E substitutions according to the Kabat numbering system; or
a lambda light chain comprising K166E, K187E, R190E, and K207E substitutions according to the Kabat numbering system.

4. The monoclonal antibody of claim 1, wherein the antibody is an IgG1 and comprises:
a heavy chain comprising K129E, K222E, and K228E substitutions according to the Kabat numbering system; and
a kappa light chain comprising K126E, K145E, K188E, K190E substitutions according to the Kabat numbering system or a lambda light chain comprising K166E, K187E, R190E, and K207E substitutions according to the Kabat numbering system.

5. The monoclonal antibody of claim 1, comprising a recombinant constant domain comprising a modification that increases binding to the neonatal Fc receptor.

6. The monoclonal antibody of claim 5, wherein the recombinant constant domain is an IgG1 constant domain comprising M428L and N434S mutations according to the EU numbering system.

7. The monoclonal antibody of claim 1, wherein the monoclinal antibody is an IgG1 an comprises a heavy chain constant domain comprising the amino acid sequence set forth as SEQ ID NO: 114, and a light chain constant domain comprising the amino acid sequence set forth as SEQ ID NO: 115.

8. The monoclonal antibody of claim 1, wherein the monoclinal antibody is an IgG1 an comprises a heavy chain constant domain comprising the amino acid sequence set forth as SEQ ID NO: 114, and a light chain constant domain comprising the amino acid sequence set forth as SEQ ID NO: 116.

9. The monoclonal antibody of claim 1, comprising a heavy chain and a light chain comprising amino acid sequences set forth as any one of:
(1) SEQ ID NOs: 60 and 61, respectively (VRC07-523LS.cv34);
(2) SEQ ID NOs: 15 and 16, respectively (VRC07-523LS.v26);
(3) SEQ ID NOs: 3 and 6, respectively (VRC07-523LS.v11);
(4) SEQ ID NOs: 8 and 6, respectively (VRC07-523LS.v14);
(5) SEQ ID NOs: 11 and 12, respectively (VRC07-523LS.v21);
(6) SEQ ID NOs: 18 and 16, respectively (VRC07-523LS.v32);
(7) SEQ ID NOs: 18 and 12, respectively (VRC07-523LS.v34);
(8) SEQ ID NOs: 25 and 26, respectively (N6LS.C1);
(9) SEQ ID NOs: 21 and 28, respectively (N6LS.15);
(10) SEQ ID NOs: 31 and 32, respectively (N6LS.30);
(11) SEQ ID NOs: 31 and 28, respectively (N6LS.35);
(12) SEQ ID NOs: 21 and 34, respectively (N6LS.47);
(13) SEQ ID NOs: 36 and 34, respectively (N6LS.49);
(13a) SEQ ID NO: 64 and 97, respectively (N6LS.cv49);
(14) SEQ ID NOs: 38 and 34, respectively (N6LS.51);
(15) SEQ ID NOs: 40 and 34, respectively (N6LS.58);
(16) SEQ ID NO: 86 and 87, respectively (N6LS.ATS8);
(17) SEQ ID NOs: 46 and 44, respectively (10E8v4-5RLS.C12);
(18) SEQ ID NOs: 46 and 48, respectively (10E8v4-5RLS.C13);
(19) SEQ ID NOs: 46 and 50, respectively (10E8v4-5RLS.C14);
(20) SEQ ID NOs: 52 and 44, respectively (10E8v4-5RLS.C15);
(21) SEQ ID NOs: 53 and 44, respectively (10E8v4-5RLS.C18);
(22) SEQ ID NOs: 56 and 44, respectively (10E8v4-5RLS.C24);
(23) SEQ ID NO: 67 and 68, respectively (10E8v4-5RLS.C27);
(24) SEQ ID NO: 67 and 70, respectively (10E8v4-5RLS.C30);
(24a) SEQ ID NO: 75 and 76, respectively (10E8v4-5RLS.cv30);
(25) SEQ ID NO: 94 and 93, respectively (10E8.ATS13);
(26) SEQ ID NO: 78 and 58, respectively (VRC01.23LS.cv1);
(27) SEQ ID NO: 60 and 61, respectively (VRC01.23LS.cv34); or
(28) SEQ ID NO: 96 and 83, respectively (VRC01.23LS.ATS5); and
wherein the antibody specifically binds to HIV-1 Env and neutralizes HIV-1.

10. A monoclonal antibody, comprising a heavy chain and a light chain comprising amino acid sequences set forth as any one of:
SEQ ID NOs: 57 and 58, respectively (VRC07-523LS.cv1);
SEQ ID NOs: 62 and 63, respectively (N6LS.cv1);
SEQ ID NO: 43 and 71, respectively (10E8v4.cc11);
SEQ ID NO: 72 and 44, respectively (10E8v4.cc16); or
SEQ ID NO: 73 and 74, respectively (10E8v4-5RLS.cv1).

11. The monoclonal antibody of claim 1, further comprising an alpha-synuclein (ATSα) domain comprising or consisting of the amino acid sequence set forth as DPDNEAYEMPSEEGYQDYEPEA (SEQ ID NO: 99) fused to the C-terminus of the light chain, the C-terminus of the heavy chain, or both the C-terminus of the light chain and the C-terminus of the heavy chain. ATSα

12. The monoclonal antibody of claim 11, wherein the heavy chain and the light chain fused to ATSα domain comprise amino acid sequences set forth as any one of:
SEQ ID NO: 3 and 79, respectively (VRC07-523LS.ATS1);
SEQ ID NO: 80 and 81, respectively (VRC07-523LS.ATS4);
SEQ ID NO: 82 and 83, respectively (VRC07-523LS.ATS10);
SEQ ID NO: 84 and 85, respectively (N6LS.ATS4);
SEQ ID NO: 86 and 87, respectively (N6LS.ATS8);
SEQ ID NO: 88 and 89, respectively (N6LS.ATS9);
SEQ ID NO: 90 and 91, respectively (10E8.ATS4);
SEQ ID NO: 92 and 93, respectively (10E8.ATS11);
SEQ ID NO: 94 and 93, respectively (10E8.ATS13);
SEQ ID NO: 95 and 79, respectively (VRC01.23LS.ATS1);
SEQ ID NO: 96 and 79, respectively (VRC01.23LS.ATS4); or
SEQ ID NO: 96 and 83, respectively (VRC01.23LS.ATS5).

13. An antigen binding fragment of the monoclonal antibody of claim 1.

14. The antigen binding fragment of claim 13, wherein the antigen binding fragment is a Fv, Fab, F(ab′)₂, scFV or a scFV₂ fragment.

15. A multispecific antibody comprising the isolated monoclonal antibody or antigen binding fragment of claim 1.

16. The multispecific antibody of claim 15, wherein the multispecific antibody is a bispecific antibody or a trispecific antibody.

17. The monoclonal antibody or antigen binding fragment or multispecific antibody of claim 1, linked to an effector molecule or a detectable marker.

18. An isolated nucleic acid molecule encoding the monoclonal antibody or antigen binding fragment or multispecific antibody of claim 1.

19. An isolated nucleic acid molecule encoding the V_{H} and/or the V_{L} of the monoclonal antibody or antigen binding fragment of claim 1.

20. The isolated nucleic acid molecule of claim 18, operably linked to a promoter.

21. The nucleic acid molecule of claim 18, wherein the nucleic acid molecule is an RNA molecule.

22. An expression vector comprising the nucleic acid molecule of claim 18.

23. The expression vector of claim 22, wherein the expression vector is a viral vector.

24. A pharmaceutical composition, comprising:
a therapeutically effective amount of the antibody, antigen binding fragment, nucleic acid molecule, or expression vector of claim 1; and
a pharmaceutically acceptable carrier.

25. A method of producing an antibody that specifically binds to HIV-1 Env, comprising:
expressing the nucleic acid molecule or expression vector of claim 18 in a host cell to produce the antibody in the host cell; and
purifying the antibody.

26. A method of detecting an HIV-1 infection in a subject, comprising:
contacting a biological sample from the subject with the antibody or antigen binding fragment of claim 1 under conditions sufficient to form an immune complex; and
detecting the presence of the immune complex in the sample, wherein the presence of the immune complex in the sample indicates that the subject has the HIV-1 infection.

27. A method of inhibiting an HIV-1 infection in a subject, comprising administering to the subject a therapeutically effective amount of the antibody, antigen binding fragment, nucleic acid molecule, expression vector, or pharmaceutical composition of claim 1 thereby preventing or treating the HIV-1 infection.

28. The method of claim 27, wherein the subject is at risk of or has an HIV-1 infection.

29. (canceled)

30. A human IgG1 monoclonal antibody comprising
(A) a heavy chain comprising K129E, K222E, and K228E substitutions according to the Kabat numbering system; and/or
a kappa light chain comprising K126E, K145E, K188E, K190E substitutions according to the Kabat numbering system; or
a lambda light chain comprising K166E, K187E, R190E, and K207E substitutions according to the Kabat numbering system; or
(B) a heavy chain comprising K129E, K222E, and K228E substitutions according to the Kabat numbering system; and
a kappa light chain comprising K126E, K145E, K188E, K190E substitutions according to the Kabat numbering system or a lambda light chain comprising K166E, K187E, R190E, and K207E substitutions according to the Kabat numbering system.

31.-32. (canceled)

33. An isolated nucleic acid molecule encoding the monoclonal antibody of claim 30 or the V_{H} or the V_{L} of the monoclonal antibody.

34.-36. (canceled)

37. An expression vector comprising the nucleic acid molecule of claim 33.

38. (canceled)

39. A pharmaceutical composition, comprising:
a therapeutically effective amount of the antibody, nucleic acid molecule, or expression vector of claim 30; and
a pharmaceutically acceptable carrier.

40. A method of producing an antibody, comprising:
expressing the nucleic acid molecule or expression vector of claim 33 in a host cell to produce the antibody in the host cell; and
purifying the antibody.


SEQUENCE LISTING (116 sequences)

SEQ ID NO: 1 - protein, 132 aa, synthetic construct
  REGION 1..132: note = Recombinant antibody sequence
QVRLSQSGGQMKKPGDSMRISCRASGYEFINCPINWIRLAPGKRPEWMGWMKPRFGAVSYARQLQGRVTMTRQLSQDPDDPDWGTAFLELRSLTSDDTAVYFCTRGKYCTARDYYNWDFEHWGQGTPVTVSS

SEQ ID NO: 2 - protein, 100 aa, synthetic construct
  REGION 1..100: note = Recombinant antibody sequence
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIK

SEQ ID NO: 3 - protein, 462 aa, synthetic construct
  REGION 1..462: note = Recombinant antibody sequence
QVRLSQSGGQMKKPGDSMRISCRASGYEFINCPINWIRLAPGKRPEWMGWMKPRFGAVSYARQLQGRVTMTRQLSQDPDDPDWGTAFLELRSLTSDDTAVYFCTRGKYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 4 - protein, 207 aa, synthetic construct
  REGION 1..207: note = Recombinant antibody sequence
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 100 aa, synthetic construct
  REGION 1..100: note = Recombinant antibody sequence
LTQSPGTLSLSPGETAIISCDTSQYGSLAWYQQRPGQAPRLVIYSGSTRAAGIPDRFSGSRWGPDYNLTISNLESGDFGVYYCQQYEFFGQGTKVQVDIK

SEQ ID NO: 6 - protein, 207 aa, synthetic construct
  REGION 1..207: note = Recombinant antibody sequence
LTQSPGTLSLSPGETAIISCDTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 132 aa, synthetic construct
  REGION 1..132: note = Recombinant antibody sequence
QVRLSQSGGQMKKPGDSMRISCDASGYEFINCPINWIRLAPGKRPEWMGWMKPRFGAVSYARQLQGRVTMTRQLSQDPDDPDWGTAFLELRSLTSDDTAVYFCTRGKYCTARDYYNWDFEHWGQGTPVTVSS

SEQ ID NO: 8 - protein, 462 aa, synthetic construct
  REGION 1..462: note = Recombinant antibody sequence
QVRLSQSGGQMKKPGDSMRISCDASGYEFINCPINWIRLAPGKRPEWMGWMKPRFGAVSYARQLQGRVTMTRQLSQDPDDPDWGTAFLELRSLTSDDTAVYFCTRGKYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 9 - protein, 132 aa, synthetic construct
  REGION 1..132: note = Recombinant antibody sequence
QVRLSQSGGQMKKPGDSMRISCDASGYEFINCPINWIRLAPGKRPEWMGWMKPRFGAVSYARQLQGRVTMTRQLSQDPDDPDWGTAFLELRSLTSDDTAVYFCTRGEYCTARDYYNWDFEHWGQGTPVTVSS

SEQ ID NO: 10 - protein, 100 aa, synthetic construct
  REGION 1..100: note = Recombinant antibody sequence
LTQSPGTLSLSPGETAIISCETSQYGSLAWYQQRPGQAPRLVIYSGSTRAAGIPDRFSGSRWGPDYNLTISNLESGDFGVYYCQQYEFFGQGTKVQVDIK

SEQ ID NO: 11 - protein, 462 aa, synthetic construct
  REGION 1..462: note = Recombinant antibody sequence
QVRLSQSGGQMKKPGDSMRISCDASGYEFINCPINWIRLAPGKRPEWMGWMKPRFGAVSYARQLQGRVTMTRQLSQDPDDPDWGTAFLELRSLTSDDTAVYFCTRGEYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 12 - protein, 207 aa, synthetic construct
  REGION 1..207: note = Recombinant antibody sequence
LTQSPGTLSLSPGETAIISCETSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 132 aa, synthetic construct
  REGION 1..132: note = Recombinant antibody sequence
QVRLSQSGGQMKKPGDSMRISCSASGYEFINCPINWIRLAPGKRPEWMGWMKPRFGAVSYARQLQGRVTMTRQLSQDPDDPDWGTAFLELRSLTSDDTAVYFCTRGKYCTARDYYNWDFEHWGQGTPVTVSS

SEQ ID NO: 14 - protein, 100 aa, synthetic construct
  REGION 1..100: note = Recombinant antibody sequence
LTQSPGTLSLSPGETAIISCSTSQYGSLAWYQQRPGQAPRLVIYSGSTRAAGIPDRFSGSRWGPDYNLTISNLESGDFGVYYCQQYEFFGQGTKVQVDIK

SEQ ID NO: 15 - protein, 462 aa, synthetic construct
  REGION 1..462: note = Recombinant antibody sequence
QVRLSQSGGQMKKPGDSMRISCSASGYEFINCPINWIRLAPGKRPEWMGWMKPRFGAVSYARQLQGRVTMTRQLSQDPDDPDWGTAFLELRSLTSDDTAVYFCTRGKYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 16 - protein, 207 aa, synthetic construct
  REGION 1..207: note = Recombinant antibody sequence
LTQSPGTLSLSPGETAIISCSTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 132 aa, synthetic construct
  REGION 1..132: note = Recombinant antibody sequence
QVRLSQSGGQMKKPGDSMRISCSASGYEFINCPINWIRLAPGKRPEWMGWMKPRFGAVSYARQLQGRVTMTRQLSQDPDDPDWGTAFLELRSLTSDDTAVYFCTRGSYCTARDYYNWDFEHWGQGTPVTVSS

SEQ ID NO: 18 - protein, 462 aa, synthetic construct
  REGION 1..462: note = Recombinant antibody sequence
QVRLSQSGGQMKKPGDSMRISCSASGYEFINCPINWIRLAPGKRPEWMGWMKPRFGAVSYARQLQGRVTMTRQLSQDPDDPDWGTAFLELRSLTSDDTAVYFCTRGSYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 19 - protein, 129 aa, synthetic construct
  REGION 1..129: note = Recombinant antibody sequence
RAHLVQSGTAMKKPGASVRVSCQTSGYTFTAHILFWFRQAPGRGLEWVGWIKPQYGAVNFGGGFRDRVTLTRQLSQDPDDPDWGIAYMDIRGLKPDDTAVYYCARDRSYGDSSWALDAWGQGTTVVVSA

SEQ ID NO: 20 - protein, 103 aa, synthetic construct [sequence omitted]

SEQ ID NO: 21 - protein, 459 aa, synthetic construct
  REGION 1..459: note = Recombinant antibody sequence
RAHLVQSGTAMKKPGASVRVSCQTSGYTFTAHILFWFRQAPGRGLEWVGWIKPQYGAVNFGGGFRDRVTLTRQLSQDPDDPDWGIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 22 - protein, 210 aa, synthetic construct
  REGION 1..210: note = Recombinant antibody sequence
YIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 129 aa, synthetic construct
  REGION 1..129: note = Recombinant antibody sequence
RAHLVQSGTAMKKPGASVRVSCQTSGYTFTAHILFWFRQAPGRGLEWVGWIKPQYGAVNFGGGFRDRVTLTRQLSQDPDDPDWGIAYMDIDGLKPDDTAVYYCARDRSYGDSSWALDAWGQGTTVVVSA

SEQ ID NO: 24 - protein, 103 aa, synthetic construct
  REGION 1..103: note = Recombinant antibody sequence
YIHVTQSPSSLSVSIGDDVTINCQTSQGVGSDLHWYQHKPGRAPKLLIHHTSSVEDGVPSRFSGSGFHTSFNLTISDLQADDIATYYCQVLQFFGRGSRLHIK

SEQ ID NO: 25 - protein, 459 aa, synthetic construct
  REGION 1..459: note = Recombinant antibody sequence
RAHLVQSGTAMKKPGASVRVSCQTSGYTFTAHILFWFRQAPGRGLEWVGWIKPQYGAVNFGGGFRDRVTLTRQLSQDPDDPDWGIAYMDIDGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 26 - protein, 210 aa, synthetic construct
  REGION 1..210: note = Recombinant antibody sequence
YIHVTQSPSSLSVSIGDDVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 103 aa, synthetic construct
  REGION 1..103: note = Recombinant antibody sequence
YIHVTQSPSSLSVSIGDEVTINCQTSQGVGSDLHWYQHKPGEAPELLIHHTSSVEDGVPSRFSGSGFHTSFNLTISDLQADDIATYYCQVLQFFGRGSRLHIK

SEQ ID NO: 28 - protein, 210 aa, synthetic construct
  REGION 1..210: note = Recombinant antibody sequence
YIHVTQSPSSLSVSIGDEVTINCQTSQGVGSDLHWYQHKPGEAPELLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 129 aa, synthetic construct
  REGION 1..129: note = Recombinant antibody sequence
RAHLVQSGTAMKEPGASVRVSCQTSGYTFTAHILFWFRQAPGRGLEWVGWIKPQYGAVNFGGGFRDRVTLTRQLSQDPDDPDWGIAYMDIRGLKPDDTAVYYCARDRSYGDSSWALDAWGQGTTVVVSA

SEQ ID NO: 30 - protein, 103 aa, synthetic construct
  REGION 1..103: note = Recombinant antibody sequence
YIHVTQSPSSLSVSIGDEVTINCQTSQGVGSDLHWYQHKPGRAPKLLIHHTSSVEDGVPSRFSGSGFHTSFNLTISDLQADDIATYYCQVLQFFGRGSRLHIK

SEQ ID NO: 31 - protein, 459 aa, synthetic construct
  REGION 1..459: note = Recombinant antibody sequence
RAHLVQSGTAMKEPGASVRVSCQTSGYTFTAHILFWFRQAPGRGLEWVGWIKPQYGAVNFGGGFRDRVTLTRQLSQDPDDPDWGIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 32 - protein, 210 aa, synthetic construct
  REGION 1..210: note = Recombinant antibody sequence
YIHVTQSPSSLSVSIGDEVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 103 aa, synthetic construct
  REGION 1..103: note = Recombinant antibody sequence
YIHVTQSPSSLSVSIGDQVTINCQTSQGVGSDLHWYQHKPGRAPKLLIHHTSSVEDGVPSRFSGSGFHTSFNLTISDLQADDIATYYCQVLQFFGRGSRLHIK

SEQ ID NO: 34 - protein, 210 aa, synthetic construct
  REGION 1..210: note = Recombinant antibody sequence
YIHVTQSPSSLSVSIGDQVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 129 aa, synthetic construct
  REGION 1..129: note = Recombinant antibody sequence
RAHLVQSGTAMKKPGASVRVSCQTSGYTFTAHILFWFRQAPGRGLEWVGWIKPQYGAVNFGGGFRDRVTLTRQLSQDPDDPDWGIAYMDIQGLKPDDTAVYYCARDRSYGDSSWALDAWGQGTTVVVSA

SEQ ID NO: 36 - protein, 459 aa, synthetic construct
  REGION 1..459: note = Recombinant antibody sequence
RAHLVQSGTAMKKPGASVRVSCQTSGYTFTAHILFWFRQAPGRGLEWVGWIKPQYGAVNFGGGFRDRVTLTRQLSQDPDDPDWGIAYMDIQGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 37 - protein, 129 aa, synthetic construct
  REGION 1..129: note = Recombinant antibody sequence
RAHLVQSGTAMKQPGASVRVSCQTSGYTFTAHILFWFRQAPGRGLEWVGWIKPQYGAVNFGGGFRDRVTLTRQLSQDPDDPDWGIAYMDIRGLKPDDTAVYYCARDRSYGDSSWALDAWGQGTTVVVSA

SEQ ID NO: 38 - protein, 459 aa, synthetic construct
  REGION 1..459: note = Recombinant antibody sequence
RAHLVQSGTAMKQPGASVRVSCQTSGYTFTAHILFWFRQAPGRGLEWVGWIKPQYGAVNFGGGFRDRVTLTRQLSQDPDDPDWGIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 39 - protein, 129 aa, synthetic construct
  REGION 1..129: note = Recombinant antibody sequence
RAHLVQSGTAMKKPGASVRVSCQTSGYTFTAHILFWFRQAPGQGLEWVGWIKPQYGAVNFGGGFRDRVTLTRQLSQDPDDPDWGIAYMDIRGLKPDDTAVYYCARDRSYGDSSWALDAWGQGTTVVVSA

SEQ ID NO: 40 - protein, 459 aa, synthetic construct
  REGION 1..459: note = Recombinant antibody sequence
RAHLVQSGTAMKKPGASVRVSCQTSGYTFTAHILFWFRQAPGQGLEWVGWIKPQYGAVNFGGGFRDRVTLTRQLSQDPDDPDWGIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 41 - protein, 131 aa, synthetic construct
  REGION 1..131: note = Recombinant antibody sequence
EVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSS

SEQ ID NO: 42 - protein, 109 aa, synthetic construct
  REGION 1..109: note = Recombinant antibody sequence
ASELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVL

SEQ ID NO: 43 - protein, 461 aa, synthetic construct
  REGION 1..461: note = Recombinant antibody sequence
EVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 44 - protein, 215 aa, synthetic construct
  REGION 1..215: note = Recombinant antibody sequence
ASELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 45 - protein, 131 aa, synthetic construct
  REGION 1..131: note = Recombinant antibody sequence
EVRLRESGGGLVEPGGSLRLSCSASGFDFDNAWMTWVRQPPGEGLEWVGRITGPGEGWSVDYAESVKGRFTISRDNTKNTLYLEMNNVRTEDTGYYFCARTGKYYDFWSGYPPGEEYFQDWGQGTLVIVSS

SEQ ID NO: 46 - protein, 461 aa, synthetic construct
  REGION 1..461: note = Recombinant antibody sequence
EVRLRESGGGLVEPGGSLRLSCSASGFDFDNAWMTWVRQPPGEGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 47 - protein, 109 aa, synthetic construct
  REGION 1..109: note = Recombinant antibody sequence
ASELTQDPAVSVALEQTVTITCRGDSLRSHYASWYQKKPGQAPVLLFYGKNNRPSGIPDRFSGSASGNRASLTITGAQAEDEADYYCSSRDKSGSRLSVFGGGTKLTVL

SEQ ID NO: 48 - protein, 215 aa, synthetic construct
  REGION 1..215: note = Recombinant antibody sequence
ASELTQDPAVSVALEQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 49 - protein, 109 aa, synthetic construct
  REGION 1..109: note = Recombinant antibody sequence
ASELTQDPAVSVALKQTVTITCRGDSLRSHYASWYQKKPGQAPVLLFYGKNNRPSGIPDRFSGSASGNRASLTITGAQAEDEADYYCSSRDESGSRLSVFGGGTKLTVL

SEQ ID NO: 50 - protein, 215 aa, synthetic construct
  REGION 1..215: note = Recombinant antibody sequence
ASELTQDPAVSVALKQTVTITCRGDSLRSHYASWYQKKPGQAPVLLFYGKNNRPSGIPDRFSGSASGNRASLTITGAQAEDEADYYCSSRDESGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 51 - protein, 131 aa, synthetic construct
  REGION 1..131: note = Recombinant antibody sequence
EVELRESGGGLVKPGGSLRLSCSASGFDFDNAWMTWVRQPPGKGLEWVGRITGPGEGWSVDYAESVKGRFTISRDNTKNTLYLEMNNVETEDTGYYFCARTGKYYDFWSGYPPGEEYFQDWGQGTLVIVSS

SEQ ID NO: 52 - protein, 461 aa, synthetic construct
  REGION 1..461: note = Recombinant antibody sequence
EVELRESGGGLVKPGGSLRLSCSASGFDFDNAWMTWVRQPPGKGLEWVGRITGPGEGWSVDYAESVKGRFTISRDNTKNTLYLEMNNVETEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 53 - protein, 131 aa, synthetic construct
  REGION 1..131: note = Recombinant antibody sequence
EVELRESGGGLVKPGGSLRLSCSASGFDFDNAWMTWVRQPPGEGLEWVGRITGPGEGWSVDYAESVKGRFTISRDNTKNTLYLEMNNVETEDTGYYFCARTGKYYDFWSGYPPGEEYFQDWGQGTLVIVSS

SEQ ID NO: 54 - protein, 461 aa, synthetic construct
  REGION 1..461: note = Recombinant antibody sequence
EVELRESGGGLVKPGGSLRLSCSASGFDFDNAWMTWVRQPPGEGLEWVGRITGPGEGWSVDYAESVKGRFTISRDNTKNTLYLEMNNVETEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 55 - protein, 131 aa, synthetic construct
  REGION 1..131: note = Recombinant antibody sequence
EVELRESGGGLVEPGGSLRLSCSASGFDFDNAWMTWVRQPPGEGLEWVGRITGPGEGWSVDYAESVKGRFTISRDNTKNTLYLEMNNVETEDTGYYFCARTGKYYDFWSGYPPGEEYFQDWGQGTLVIVSS

SEQ ID NO: 56 - protein, 461 aa, synthetic construct
  REGION 1..461: note = Recombinant antibody sequence
EVELRESGGGLVEPGGSLRLSCSASGFDFDNAWMTWVRQPPGEGLEWVGRITGPGEGWSVDYAESVKGRFTISRDNTKNTLYLEMNNVETEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 57 - protein, 462 aa, synthetic construct
  REGION 1..462: note = Recombinant antibody sequence
QVRLSQSGGQMKKPGDSMRISCRASGYEFINCPINWIRLAPGKRPEWMGWMKPRFGAVSYARQLQGRVTMTRQLSQDPDDPDWGTAFLELRSLTSDDTAVYFCTRGKYCTARDYYNWDFEHWGQGTPVTVSSASTKGPSVFPLAPSSESTSGGTAALGCLVKDYFPEPVTVSWNSGALTSGVHTFPAVLQSSGLYSLSSVVTVPSSSLGTQTYICNVNHKPSNTKVDKEVEPESCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 58 - protein, 207 aa, synthetic construct
  REGION 1..207: note = Recombinant antibody sequence
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLESGTASVVCLLNNFYPREAEVQWKVDNALQSGNSQESVTEQDSKDSTYSLSSTLTLSKADYEEHEVYACEVTHQGLSSPVTKSFNRGEC

SEQ ID NO: 59 - protein, 132 aa, synthetic construct; identical to SEQ ID NO: 17
  REGION 1..132: note = Recombinant antibody sequence

SEQ ID NO: 60 - protein, 462 aa, synthetic construct
  REGION 1..462: note = Recombinant antibody sequence
QVRLSQSGGQMKKPGDSMRISCSASGYEFINCPINWIRLAPGKRPEWMGWMKPRFGAVSYARQLQGRVTMTRQLSQDPDDPDWGTAFLELRSLTSDDTAVYFCTRGSYCTARDYYNWDFEHWGQGTPVTVSSASTKGPSVFPLAPSSESTSGGTAALGCLVKDYFPEPVTVSWNSGALTSGVHTFPAVLQSSGLYSLSSVVTVPSSSLGTQTYICNVNHKPSNTKVDKEVEPESCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 61 - protein, 207 aa, synthetic construct
  REGION 1..207: note = Recombinant antibody sequence
LTQSPGTLSLSPGETAIISCETSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLESGTASVVCLLNNFYPREAEVQWKVDNALQSGNSQESVTEQDSKDSTYSLSSTLTLSKADYEEHEVYACEVTHQGLSSPVTKSFNRGEC

SEQ ID NO: 62 - protein, 459 aa, synthetic construct
  REGION 1..459: note = Recombinant antibody sequence
RAHLVQSGTAMKKPGASVRVSCQTSGYTFTAHILFWFRQAPGRGLEWVGWIKPQYGAVNFGGGFRDRVTLTRQLSQDPDDPDWGIAYMDIRGLKPDDTAVYYCARDRSYGDSSWALDAWGQGTTVVVSAASTKGPSVFPLAPSSESTSGGTAALGCLVKDYFPEPVTVSWNSGALTSGVHTFPAVLQSSGLYSLSSVVTVPSSSLGTQTYICNVNHKPSNTKVDKEVEPESCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 63 - protein, 210 aa, synthetic construct
  REGION 1..210: note = Recombinant antibody sequence
YIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLESGTASVVCLLNNFYPREAEVQWKVDNALQSGNSQESVTEQDSKDSTYSLSSTLTLSKADYEEHEVYACEVTHQGLSSPVTKSFNRGEC

SEQ ID NO: 64 - protein, 459 aa, synthetic construct
  REGION 1..459: note = Recombinant antibody sequence
RAHLVQSGTAMKKPGASVRVSCQTSGYTFTAHILFWFRQAPGRGLEWVGWIKPQYGAVNFGGGFRDRVTLTRQLSQDPDDPDWGIAYMDIQGLKPDDTAVYYCARDRSYGDSSWALDAWGQGTTVVVSAASTKGPSVFPLAPSSESTSGGTAALGCLVKDYFPEPVTVSWNSGALTSGVHTFPAVLQSSGLYSLSSVVTVPSSSLGTQTYICNVNHKPSNTKVDKEVEPESCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 65 - protein, 131 aa, synthetic construct
  REGION 1..131: note = Recombinant antibody sequence
EVRLRESGGGLVSPGGSLRLSCSASGFDFDNAWMTWVRQPPGSGLEWVGRITGPGEGWSVDYAESVKGRFTISRDNTKNTLYLEMNNVRTEDTGYYFCARTGKYYDFWSGYPPGEEYFQDWGQGTLVIVSS

SEQ ID NO: 66 - protein, 109 aa, synthetic construct
  REGION 1..109: note = Recombinant antibody sequence
ASELTQDPAVSVALSQTVTITCRGDSLRSHYASWYQKKPGQAPVLLFYGKNNRPSGIPDRFSGSASGNRASLTITGAQAEDEADYYCSSRDKSGSRLSVFGGGTKLTVL

SEQ ID NO: 67 - protein, 461 aa, synthetic construct
  REGION 1..461: note = Recombinant antibody sequence
EVRLRESGGGLVSPGGSLRLSCSASGFDFDNAWMTWVRQPPGSGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 68 - protein, 215 aa, synthetic construct
  REGION 1..215: note = Recombinant antibody sequence
ASELTQDPAVSVALSQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 69 - protein, 109 aa, synthetic construct
  REGION 1..109: note = Recombinant antibody sequence
ASELTQDPAVSVALKQTVTITCRGDSLRSHYASWYQKKPGQAPVLLFYGKNNRPSGIPDRFSGSASGNRASLTITGAQAEDEADYYCSSRDQSGSRLSVFGGGTKLTVL

SEQ ID NO: 70 - protein, 215 aa, synthetic construct
  REGION 1..215: note = Recombinant antibody sequence
ASELTQDPAVSVALKQTVTITCRGDSLRSHYASWYQKKPGQAPVLLFYGKNNRPSGIPDRFSGSASGNRASLTITGAQAEDEADYYCSSRDQSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 71 - protein, 215 aa, synthetic construct
  REGION 1..215: note = Recombinant antibody sequence
ASELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPEAAPSVTLFPPSSEELQANEATLVCLISDFYPGAVTVAWKADSSPVEAGVETTTPSEQSNNKYAASSYLSLTPEQWESHESYSCQVTHEGSTVEKTVAPTECS

SEQ ID NO: 72 - protein, 461 aa, synthetic construct
  REGION 1..461: note = Recombinant antibody sequence
EVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSEETEGPSVFPLAPSSESTEGGTAALGCLVKDYFPEPVTVSWNSGELTSGVHTFPAVLQSSGLYSLSSVVTVPSSSLGEETYICNVNHEPSNTEVDKEVEPESCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 73 - protein, 461 aa, synthetic construct
  REGION 1..461: note = Recombinant antibody sequence
EVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSESTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 74 - protein, 215 aa, synthetic construct
  REGION 1..215: note = Recombinant antibody sequence
ASELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSEQSNNKYAASSYLSLTPEQWESHESYSCQVTHEGSTVEETVAPTECS

SEQ ID NO: 75 - protein, 461 aa, synthetic construct
  REGION 1..461: note = Recombinant antibody sequence
EVRLRESGGGLVSPGGSLRLSCSASGFDFDNAWMTWVRQPPGSGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSESTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 76 - protein, 215 aa, synthetic construct
  REGION 1..215: note = Recombinant antibody sequence
ASELTQDPAVSVALKQTVTITCRGDSLRSHYASWYQKKPGQAPVLLFYGKNNRPSGIPDRFSGSASGNRASLTITGAQAEDEADYYCSSRDQSGSRLSVFGGGTKLTVLSQPKAAPSVTLFPPSSEELQANKATLVCLISDFYPGAVTVAWKADSSPVKAGVETTTPSEQSNNKYAASSYLSLTPEQWESHESYSCQVTHEGSTVEETVAPTECS

SEQ ID NO: 77 - protein, 128 aa, synthetic construct
  REGION 1..128: note = Recombinant antibody sequence
QVQLVQSGGQMKKPGESMRISCRASGYEFIDCTLNWIRLAPGKRPEWMGWLKPRWGAVNYARPLQGRVTMTRQLSQDPDDPDWGTAFLELRSLTVDDTAVYFCTRGKNCDYNWDFEHWGRGTPVIVSS

SEQ ID NO: 78 - protein, 458 aa, synthetic construct
  REGION 1..458: note = Recombinant antibody sequence
QVQLVQSGGQMKKPGESMRISCRASGYEFIDCTLNWIRLAPGKRPEWMGWLKPRWGAVNYARPLQGRVTMTRQLSQDPDDPDWGTAFLELRSLTVDDTAVYFCTRGKNCDYNWDFEHWGRGTPVIVSSASTKGPSVFPLAPSSESTSGGTAALGCLVKDYFPEPVTVSWNSGALTSGVHTFPAVLQSSGLYSLSSVVTVPSSSLGTQTYICNVNHKPSNTKVDKEVEPESCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 79 - protein, 231 aa, synthetic construct
  REGION 1..231: note = Recombinant antibody sequence
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGDPDNEAYEMPSEEGYQDYEPEA

SEQ ID NO: 80 - protein, 486 aa, synthetic construct
  REGION 1..486: note = Recombinant antibody sequence
QVRLSQSGGQMKKPGDSMRISCRASGYEFINCPINWIRLAPGKRPEWMGWMKPRFGAVSYARQLQGRVTMTRQLSQDPDDPDWGTAFLELRSLTSDDTAVYFCTRGKYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGDPDNEAYEMPSEEGYQDYEPEA

SEQ ID NO: 81 - protein, 231 aa, synthetic construct; identical to SEQ ID NO: 79
  REGION 1..231: note = Recombinant antibody sequence

SEQ ID NO: 82 - protein, 486 aa, synthetic construct
  REGION 1..486: note = Recombinant antibody sequence
QVRLSQSGGQMKKPGDSMRISCSASGYEFINCPINWIRLAPGKRPEWMGWMKPRFGAVSYARQLQGRVTMTRQLSQDPDDPDWGTAFLELRSLTSDDTAVYFCTRGSYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGDPDNEAYEMPSEEGYQDYEPEA

SEQ ID NO: 83 - protein, 231 aa, synthetic construct
  REGION 1..231: note = Recombinant antibody sequence
LTQSPGTLSLSPGETAIISCETSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGDPDNEAYEMPSEEGYQDYEPEA

SEQ ID NO: 84 - protein, 483 aa, synthetic construct
  REGION 1..483: note = Recombinant antibody sequence
RAHLVQSGTAMKKPGASVRVSCQTSGYTFTAHILFWFRQAPGRGLEWVGWIKPQYGAVNFGGGFRDRVTLTRQLSQDPDDPDWGIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGDPDNEAYEMPSEEGYQDYEPEA

SEQ ID NO: 85 - protein, 234 aa, synthetic construct
  REGION 1..234: note = Recombinant antibody sequence
YIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGDPDNEAYEMPSEEGYQDYEPEA

SEQ ID NO: 86 - protein, 483 aa, synthetic construct
  REGION 1..483: note = Recombinant antibody sequence
RAHLVQSGTAMKKPGASVRVSCQTSGYTFTAHILFWFRQAPGRGLEWVGWIKPQYGAVNFGGGFRDRVTLTRQLSQDPDDPDWGIAYMDIDGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGDPDNEAYEMPSEEGYQDYEPEA

SEQ ID NO: 87 - protein, 234 aa, synthetic construct
  REGION 1..234: note = Recombinant antibody sequence
YIHVTQSPSSLSVSIGDQVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGDPDNEAYEMPSEEGYQDYEPEA

SEQ ID NO: 88 - protein, 483 aa, synthetic construct
  REGION 1..483: note = Recombinant antibody sequence
RAHLVQSGTAMKKPGASVRVSCQTSGYTFTAHILFWFRQAPGRGLEWVGWIKPQYGAVNFGGGFRDRVTLTRQLSQDPDDPDWGIAYMDIQGLKPDDTAVYYCARDRSYGDSSWALDAWGQGTTVVVSAASTKGPSVFPLAPSSESTSGGTAALGCLVKDYFPEPVTVSWNSGALTSGVHTFPAVLQSSGLYSLSSVVTVPSSSLGTQTYICNVNHKPSNTKVDKEVEPESCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGDPDNEAYEMPSEEGYQDYEPEA

SEQ ID NO: 89 - protein, 234 aa, synthetic construct
  REGION 1..234: note = Recombinant antibody sequence
YIHVTQSPSSLSVSIGDQVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLESGTASVVCLLNNFYPREAEVQWKVDNALQSGNSQESVTEQDSKDSTYSLSSTLTLSKADYEEHEVYACEVTHQGLSSPVTKSFNRGECGGDPDNEAYEMPSEEGYQDYEPEA

SEQ ID NO: 90 - protein, 485 aa, synthetic construct
  REGION 1..485: note = Recombinant antibody sequence
EVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGDPDNEAYEMPSEEGYQDYEPEA

SEQ ID NO: 91 - protein, 239 aa, synthetic construct
  REGION 1..239: note = Recombinant antibody sequence
ASELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGDPDNEAYEMPSEEGYQDYEPEA

SEQ ID NO: 92 - protein, 485 aa, synthetic construct
  REGION 1..485: note = Recombinant antibody sequence
EVRLRESGGGLVSPGGSLRLSCSASGFDFDNAWMTWVRQPPGSGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGDPDNEAYEMPSEEGYQDYEPEA

SEQ ID NO: 93 - protein, 239 aa, synthetic construct
  REGION 1..239: note = Recombinant antibody sequence
ASELTQDPAVSVALSQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGDPDNEAYEMPSEEGYQDYEPEA

SEQ ID NO: 94 - protein, 485 aa, synthetic construct
  REGION 1..485: note = Recombinant antibody sequence
EVRLRESGGGLVSPGGSLRLSCSASGFDFDNAWMTWVRQPPGSGLEWVGRITGPGEGWSVDYAESVKGRFTISRDNTKNTLYLEMNNVRTEDTGYYFCARTGKYYDFWFGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGDPDNEAYEMPSEEGYQDYEPEA

SEQ ID NO: 95 - protein, 458 aa, synthetic construct
  REGION 1..458: note = Recombinant antibody sequence
QVQLVQSGGQMKKPGESMRISCRASGYEFIDCTLNWIRLAPGKRPEWMGWLKPRWGAVNYARPLQGRVTMTRQLSQDPDDPDWGTAFLELRSLTVDDTAVYFCTRGKNCDYNWDFEHWGRGTPVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 96 - protein, 482 aa, synthetic construct
  REGION 1..482: note = Recombinant antibody sequence
QVQLVQSGGQMKKPGESMRISCRASGYEFIDCTLNWIRLAPGKRPEWMGWLKPRWGAVNYARPLQGRVTMTRQLSQDPDDPDWGTAFLELRSLTVDDTAVYFCTRGKNCDYNWDFEHWGRGTPVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGDPDNEAYEMPSEEGYQDYEPEA

SEQ ID NO: 97 - protein, 210 aa, synthetic construct
  REGION 1..210: note = Recombinant antibody sequence
YIHVTQSPSSLSVSIGDQVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLESGTASVVCLLNNFYPREAEVQWKVDNALQSGNSQESVTEQDSKDSTYSLSSTLTLSKADYEEHEVYACEVTHQGLSSPVTKSFNRGEC

SEQ ID NO: 98 - protein, 131 aa, synthetic construct
  REGION 1..131: note = Recombinant antibody sequence
EVRLRESGGGLVSPGGSLRLSCSASGFDFDNAWMTWVRQPPGSGLEWVGRITGPGEGWSVDYAESVKGRFTISRDNTKNTLYLEMNNVRTEDTGYYFCARTGKYYDFWFGYPPGEEYFQDWGQGTLVIVSS

SEQ ID NO: 99 - protein, 22 aa, synthetic construct
  REGION 1..22: note = Recombinant antibody sequence
DPDNEAYEMPSEEGYQDYEPEA

SEQ ID NO: 100 - protein, 856 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 101 - protein, 23 aa, synthetic construct
  REGION 1..23: note = Recombinant antibody sequence
RVTMTRDTSISTAYMELSRLRSD

SEQ ID NO: 102 - protein, 23 aa, synthetic construct
  REGION 1..23: note = Recombinant antibody sequence
RVTMTRDVYSDTAFLELRSLTVD

SEQ ID NO: 103 - protein, 23 aa, synthetic construct
  REGION 1..23: note = Recombinant antibody sequence
RVTMTRDMYSETAFLELRSLTSD

SEQ ID NO: 104 - protein, 30 aa, synthetic construct
  REGION 1..30: note = Recombinant antibody sequence
RVSMTRQLSQDPDDPDWGVAYMEFSGLTSA

SEQ ID NO: 105 - protein, 30 aa, synthetic construct
  REGION 1..30: note = Recombinant antibody sequence
RVSMSRLFSQDLYYPDRGTAYLEFSGLTSA

SEQ ID NO: 106 - protein, 30 aa, synthetic construct
  REGION 1..30: note = Recombinant antibody sequence
RVTMTRQLSQDPDDPDWGTAFLELRSLTSD

SEQ ID NO: 107 - protein, 30 aa, synthetic construct
  REGION 1..30: note = Recombinant antibody sequence
RVTMTRQLSQDPYYPDWGTAFLELRSLTSD

SEQ ID NO: 108 - protein, 30 aa, synthetic construct
  REGION 1..30: note = Recombinant antibody sequence
RVTMTRQLSQDPRRPDWGTAFLELRSLTSD

SEQ ID NO: 109 - protein, 30 aa, synthetic construct
  REGION 1..30: note = Recombinant antibody sequence
RVTMTRQLSQDPRRPRWGTAFLELRSLTSD

SEQ ID NO: 110 - protein, 30 aa, synthetic construct
  REGION 1..30: note = Recombinant antibody sequence
RVTMTRQLSQDPDDPDWGDAFLELRSLTSD

SEQ ID NO: 111 - protein, 30 aa, synthetic construct
  REGION 1..30: note = Recombinant antibody sequence
RVTMTRQLSQDPDDPDWGTADLELRSLTSD

SEQ ID NO: 112 - protein, 30 aa, synthetic construct
  REGION 1..30: note = Recombinant antibody sequence
RVTMTRQLSQDPDDPDWGDADLELRSLTSD

SEQ ID NO: 113 - protein, 15 aa, synthetic construct
  REGION 1..15: note = Recombinant antibody sequence
QLSQDPDDPDWGTAF

SEQ ID NO: 114 - protein, 330 aa, Synthetic construct
ASTKGPSVFPLAPSSESTSGGTAALGCLVKDYFPEPVTVSWNSGALTSGVHTFPAVLQSSGLYSLSSVVTVPSSSLGTQTYICNVNHKPSNTKVDKEVEPESCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 115 - protein, 207 aa, synthetic construct; identical to SEQ ID NO: 58

SEQ ID NO: 116 - protein, 215 aa, synthetic construct; identical to SEQ ID NO: 74